(19) United States
(12) Patent Application Publication
(10) Pub. No.: US 2019/0153154 A1
(43) Pub. Date: May 23, 2019

(54) POLYMERS OF INTRINSIC MICROPOROSITY (PIMS) CONTAINING LOCKED SPIROBISINDANE STRUCTURES AND METHODS OF SYNTHESIS OF PIMS POLYMERS

(71) Applicant: Auckland Uniservices Limited, Auckland (NZ)

(72) Inventors: Jianyong Jin, Auckland (NZ); Jian Zhang, Auckland Central (NZ)

(21) Appl. No.: 15/752,832

(22) PCT Filed: Aug. 22, 2016
(86) PCT No.: PCT/NZ2016/050133

§ 371 Date: Feb. 14, 2018

(30) Foreign Application Priority Data
Aug. 20, 2015 (NZ) 711240
Sep. 9, 2015 (NZ) 712115

(51) Int. Cl. C08G 65/40

(52) U.S. Cl. CPC C08G 65/4081; C07C 39/17; C08G 2650/62; C08G 65/4018

(57) ABSTRACT

A method of increasing the rigidity of PIM homo- or co-polymers including repeating units containing at least one biscatechol monomer having a fused spiro-bisindane (SBI) ring system of Formula (I), the SBI ring system including a bicyclic spiro-carbon: (I) wherein each R¹ can be the same or different, each R² can be the same or different and wherein R¹ and/or R² are selected from any one or more of H; straight or branched, saturated or unsaturated lower C₁-C₆ alkyl groups, wherein the lower alkyl groups can include aromatic or non-aromatic ring structures; R³OR⁴, R³O(C═O)R⁴, R³C(═O)OR⁴, or R³OH, wherein each of R³ and R⁴ are the same or different and are selected from straight or branched, saturated or unsaturated lower C₁-C₆ alkyl groups; and R⁵ and R represent suitable linking monomers, wherein the linking monomers can be the same or different and are preferably selected from any one or more tetrahalo aromatic monomers; and wherein the method includes the step of introducing an intra-molecular lock between C1 and C2 of the biscatechol monomer of Formula (I).

### FIELD OF THE INVENTION

The present invention relates to a method of increasing the polymer chain rigidity of high molecular weight polymers of intrinsic microporosity (PIMs or PIM polymers). The present invention also relates to novel biscatechol monomers having an intramolecular locked bicyclic spiro-carbon, to methods of synthesis of said biscatechol monomers, to novel PIM polymers containing said biscatechol monomers, and to novel monomers of use in preparing the novel biscatechol monomers having an intramolecular locked bicyclic spiro-carbon. The present invention also relates to methods of producing PIM polymers including a fluoride-mediated polymerisation method for the synthesis of PIM polymers. The present invention has potential application in gas adsorption, gas purification, gas separation membrane materials, organic sorbent materials, organic dye adsorption, and complex mixtures separation in liquid form such as organic solvent nanofiltration membrane materials.

### BACKGROUND OF THE INVENTION

High molecular weight polymers of intrinsic microporosity (PIMs or PIM polymers) were first reported in 2004 by Budd and McKeown.¹ PIM polymers are typically formed by a dibenzodioxin-forming polymerisation, in which a biscatechol monomer and an activated tetra halo-substituted aromatic are employed to give a polybenzodioxin via double aromatic nucleophilic substitution in the presence of potassium carbonate (K₂CO₃) (FIG. 1, Method A).² In 2005, Kricheldorf et al described a different PIM polymer synthetic strategy based on trimethylsilyl(TMS)-derived biscatechol (FIG. 1, Method B).³ According to their findings, the cyclic ladder oligomer and polymers were formed as the predominant product. However, this method still employed the same K₂CO₃ condition as the traditional method.

The PIM polymer structure has a contorted and rigid backbone that has a limited ability to pack efficiently in the solid state. The inefficient packing results in a large amount of free volume and therefore intrinsic porosity of the macromolecules in the solid state. The pore size of PIM polymers is usually smaller than 2 nm determined by positron annihilation lifetime spectroscopy (PALS) and low-temperature gas adsorption. According to the definition of porous materials recommended by IUPAC, based on pore size these unique polymers are a type of microporous material.

This intrinsic porosity provides PIM polymers with adsorption properties. They effectively act like a zeolite or activated carbon in their ability to take up small molecules in their pores and/or act as molecular sieves. Recent work with PIM polymers has focussed on their development for molecular separations including gas separation.⁴⁻¹¹

PIM-1¹, invented by Budd and McKeown, was the first polymer of its kind and is one of the best-known flagship PIM polymers. It has shown great potential in membrane applications, especially for gas separation. PIM-1 is formed by polymerizing a bis-catechol monomer 5,5′,6,6′-tetrahydroxy-3,3,3′,3′-tetramethyl-1,1′-spirobisindane (1) with 2,3,5,6-tetrafluoroterephthalonitrile (2) (FIG. 2). The reaction undergoes a double aryl nucleophilic substitution by using an excessive amount of K₂CO₃ as base at 65° C. for 72 hours.

As shown in FIG. 3, PIM-1 contains a biscatechol monomer having a spiro-bisindane (SBI) structural unit. A spiro compound is a bicyclic organic compound with rings connected through just one atom. The connecting atom is also called the spiro-atom, most often a quaternary carbon (“spiro-carbon”). In the SBI structural unit the spiro-carbon is a bicyclic carbon. The dihedral angles measured by the two indane planar cyclics of the SBI structural unit can fluctuate. Thus, the SBI structural unit has a relatively low barrier of movement about the bicyclic spiro-carbon. SBI was the first building block employed in the history of PIM polymer development, and contributed enormously to the further development of PIM polymers.

When individual PIM-1 polymer chains pack together, a microporous structure results. However, due to the relative flexibility of the spiro-carbon of the SBI unit, pore sizes can vary which impacts on the gas separation performance of the PIM-1 polymer in terms of gas permselectivity.

Freeman¹² discussed the theoretical relationship between structure and property for gas separation/permselectivity. That is, the inter-chain distance of polymer chains governs the permeability by introducing free volume; whereas the increase in backbone rigidity can lead to an elevated permselectivity. Gas permeability and permselectivity are two of the most important parameters to evaluate the performance of any polymers for gas separation applications. As a result, in recent years, a number of new PIM polymers have been designed and synthesized via two different strategies with the aim of increasing the rigidity of the polymer backbone structure.

The first strategy was to replace the biscatechol monomers having a bicyclic spiro-carbon with alternative, rigid building blocks that did not have the spiro-carbon. Such rigid building blocks were used to give a more rigid PIM polymer backbone. The most successful high performance building blocks utilized for this purpose include ethanoanthracene (EA), Troger's Base (TB) and triptycene (TP) (FIG. 4). EA-containing PIM polymers have shown improved gas separation over those containing biscatechol monomers with an SBI unit, for example, thus supporting the theory proposed by Freeman. Likewise PIM polymers containing the TB and TP building blocks have also exhibited improved gas separation performance in terms of permselectivity and permeability.

The second logical strategy involved changing the substituent groups about the bicyclic spiro-carbon to increase the barrier of the spiro-carbon movement. In this strategy, spiro-bisfluorene (SBF, FIG. 4), for example, was employed. SBF is more bulky than SBI, however, it retains the bicyclic spiro-carbon and therefore retains some flexibility. Incorporation of the more bulky SBF into PIM polymers resulted in improved gas separation performance over PIM polymers incorporating SBI, which can be considered as a step forward.

PIM polymers typically take the form of homo-polymers or co-polymers. PIM homo-polymers comprise a sequence of identical biscatechol monomers linked together by a suitable linking monomer. Co-polymers comprise a sequence including either two or more different types of biscatechol monomers linked together by identical, suitable linking monomers; two or more different types of linking monomers linked together by identical biscatechol monomers; or two or more different types of biscatechol monomers linked together by two or more different types of linking monomers. Where the biscatechol monomers employed include a bicyclic spiro-carbon, it creates a site of contortion in the PIM homo- and co-polymer chains. Therefore, when these chains pack together inefficiently, a microporous structure results intrinsically. However, the relative flexibility of the spiro-carbon means that individual pores within the resulting microporous structure can fluctuate in size. This limits gas permselectivity. There is therefore a need for alternative options to improve rigidity of PIM polymers including a bicyclic spiro-carbon.

Also, the original synthetic protocol developed by Budd and McKeown has limitations where certain kinds of PIM monomers, such as those containing base sensitive functional groups, are chemically unstable in the presence of K₂CO₃ (or other carbonate salts). Consequently, such PIM monomers do not polymerize and form well defined PIM polymers in the presence of K₂CO₃.

Kricheldorf et al,¹³ has developed a fluoride-mediated single nucleophilic aromatic substitution method for the synthesis of high-molecular weight polyarylethers. Fluoride-mediated single nucleophilic aromatic substitution polymerization uses mild reaction conditions and neutral condensates byproduct, however the use of fluoride-mediated substitution has never been considered for the synthesis of PIM polymers.

There is therefore also a need for alternative methods of synthesis of PIM polymers, particularly (but not exclusively) for when the PIM polymer contains base sensitive functional groups.

It is an object of the present invention to meet the above mentioned needs and/or to overcome or ameliorate the above mentioned disadvantages of the prior art. It is a further or alternative object to provide novel biscatechol monomers having a locked bicyclic spiro-carbon and PIM polymers containing said biscatechol monomers. It is also an object to provide alternative methods for producing PIM polymers. The above objects are to be read disjunctively and with the alternative object of to at least provide the public with a useful choice.

### SUMMARY OF THE INVENTION

In a first aspect, the present invention provides a method of increasing the rigidity of PIM homo- or co-polymers including repeating units containing at least one biscatechol monomer having a fused spiro-bisindane ring system of Formula (I), the spiro-bisindane ring system including a bicyclic spiro-carbon:

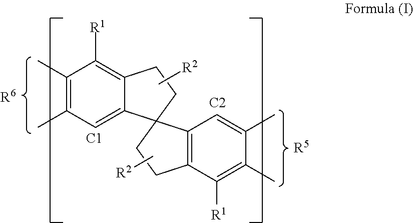

wherein

each R¹ can be the same or different, each R² can be the same or different and wherein R¹ and/or R² are selected from any one or more of H; straight or branched, saturated or unsaturated lower C₁-C₆ alkyl groups, wherein the lower alkyl groups can include aromatic or non-aromatic ring structures; R³OR⁴, R³O(C═O)R⁴, R³C(═O)OR⁴, or R³OH, wherein each of R³ and R⁴ are the same or different and are selected from straight or branched, saturated or unsaturated lower C₁-C₆ alkyl groups; and

R⁵ and R⁶ represent suitable linking monomers, wherein the linking monomers can be the same or different and are preferably selected from any one or more tetrahalo aromatic monomers;

and wherein the method includes the step of introducing an intra-molecular lock between C1 and C2 of the biscatechol monomer of Formula (I).

Preferably, each R² is dimethyl (as shown below) and the biscatechol monomer of Formula (I) is:

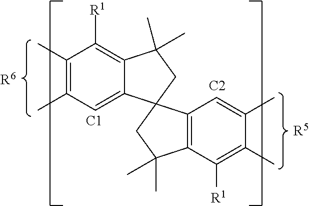

Preferably, where each R¹ is H and each R² is dimethyl (as shown above), the biscatechol monomer of Formula (I) contains a fused spiro-bisindane ring system (SBI).

Preferably, each R² is a C₆ aromatic ring (as shown below) and the biscatechol monomer of Formula (I) is:

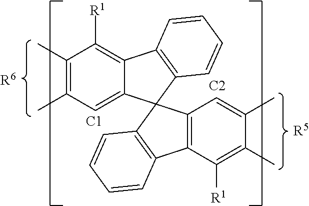

Preferably, where each R¹ is H and each R² is a C₆ aromatic ring (as shown above), the biscatechol monomer of Formula (I) contains a fused spiro-bisfluorene ring system (SBF).

Preferably, the suitable tetrahalo aromatic monomer is a tetrafluoro, tetrabromo, tetrachloro or tetraiodo aromatic monomer.

Preferably, the tetrahalo aromatic monomer is a tetrafluoro aromatic monomer.

In a second aspect, the present invention provides a method of increasing the rigidity of PIM homo- or co-polymers including repeating units containing at least one biscatechol monomer having a fused spiro-bisindane ring system of Formula (I) wherein the method includes the step of introducing an intra-molecular lock between C1 and C2 of the biscatechol monomer of Formula (I) and wherein the intramolecular lock between C1 and C2 forms:

(a) an 8 membered ring structure including —CH₂—Y—CH₂—, and wherein Y is O; CH₂; C(═O); C(OR⁷)(OR⁸); NH; NR⁹; S; S(═O); SO₂; C(═S); PH; PR¹⁰; BH; BR¹¹ or BOR¹¹; and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures;

or

(b) a 7 membered ring structure including —CH₂-CH₂—, —CH═CH—, —C(═O)O—, —C(═O)NH— or —CHR¹²-CHR¹²—, wherein R¹² are selected from any one or more of H or straight or branched, saturated or unsaturated lower C₁-C₆ alkyl groups, wherein the lower alkyl groups can include aromatic or non-aromatic ring structures.

Preferably, each R² is a dimethyl or a C₆ aromatic ring.

Preferably, the biscatechol monomer of Formula (I) contains a fused spiro-bisindane ring system (SBI) or a fused spiro-bisfluorene ring system (SBF).

In a third aspect, the present invention provides a method of preparing a biscatechol monomer of Formula (V), the biscatechol monomer including a fused spiro-bisindane ring system having a locked bicyclic spiro-carbon, wherein the method includes the steps of:
a) protecting each hydroxyl group of a dimethyl substituted biscatechol monomer of Formula (II) having a bicyclic spiro-carbon with a silyl ether protecting group to give a protected dimethyl substituted biscatechol monomer of Formula (III):

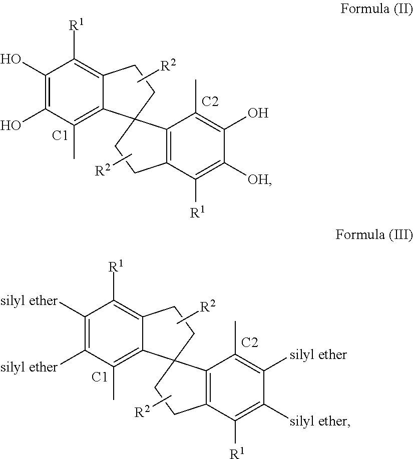

wherein the silyl ether protecting groups are the same or different; and
wherein, R¹ and R² are as defined previously for Formula (I) and
b) halogenating the silyl ether protected biscatechol monomer of Formula (III) from step (a) to form a silyl ether protected bis(halomethyl) substituted biscatechol monomer of Formula (IV), wherein the halide ion employed in the halogenation is selected from bromide, chloride or iodide ions:

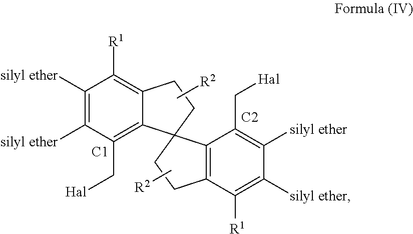

wherein R¹ and R² and silyl ether are as defined previously for Formula (III), and wherein Hal is any one of bromide, chloride or iodide ions, and
c) dehalogenating the biscatechol monomer of Formula (IV) and forming a covalent, intramolecular lock between C1 and C2 of the protected biscatechol monomer of Formula (IV) with a suitable locking group X to provide a silyl ether protected biscatechol monomer of Formula (V) having a locked bicyclic spiro-carbon:

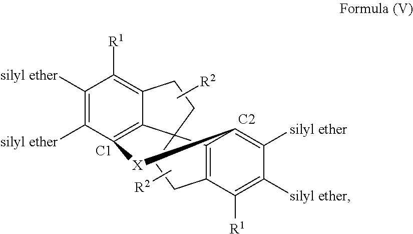

wherein, R¹ and R² and silyl ether are as defined previously for Formula (III), and
(i) X is —CH₂—Y—CH₂—, and wherein Y is O; CH₂; C(═O); C(OR⁷)(OR⁸); NH; NR⁹; S; S(═O); SO₂; C(═S); PH; PR¹⁰; BH; BR¹¹ or BOR¹¹;
   and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or
(ii) X is —CH₂-CH₂—, —CH═CH—, —(═O)O—, —C(═O)NH— or —CHR¹²-CHR¹²—, wherein R¹² are selected from any one or more of H or straight or branched, saturated or unsaturated lower C₁-C₆ alkyl groups, wherein the lower alkyl groups can include aromatic or non-aromatic ring structures.

Preferably, the silyl ether protecting group is selected from Formula (VI):

R¹³R¹⁴R¹⁵Si—O—R¹⁶ (VI)

wherein R¹³ to R¹⁶ are alkyl groups or aryl groups.

Preferably, the silyl ether protecting group is selected from any one or more of trimethylsilyl (TMS), tert-butyldiphenylsilyl (TBDPS), tert-butyldimethylsilyl (TBS) and triisopropylsilyl (TIPS).

Preferably, the silyl ether protecting group is tert-butyl dimethyl silyl (TBS).

Preferably, the dehalogenation step (c) to form an intramolecular lock between C1 and C2 is catalysed by a transition metal salt, metal oxide, and/or a pure metal.

Preferably, the transition metal salt is a silver(I), iron(III), titanium(II) or tin(II) salt.

Preferably, the silver(I) salt is AgNO₃ or Ag₂CO₃.

Preferably, the metal oxide is ZnO or Ag₂O.

Preferably, the pure metal is zinc.

Preferably, where X is —CH₂—Y—CH₂— and Y is O, the dehalogenation step (c) to form an intramolecular lock between C1 and C2 is catalysed by a silver(I) salt, more preferably Ag₂CO₃.

Alternatively, in step (c) the halide ions in Formula (IV) are each substituted by a hydroxyl group which then undergo cyclised dehydration to form the covalent, intramolecular lock, and wherein X is —CH₂—Y— CH₂—and Y is O in Formula (V).

Preferably, each R² is a dimethyl or a C₆ aromatic ring.

Preferably, the biscatechol monomer of Formula (V) contains a fused spiro-bisindane ring system (SBI) or a fused spiro-bisfluorene ring system (SBF).

In a fourth aspect, the present invention provides a method of preparing a biscatechol monomer of Formula (V), the biscatechol monomer including a fused spiro-bisindane ring system having a bicyclic spiro-carbon, wherein the method includes the steps of:
a) halogenating a silyl ether protected biscatechol monomer of Formula (III) to form a silyl ether protected bis(halomethyl) substituted biscatechol monomer of Formula (IV), wherein the halide ion employed in the halogenation is selected from bromide, chloride or iodide ions:

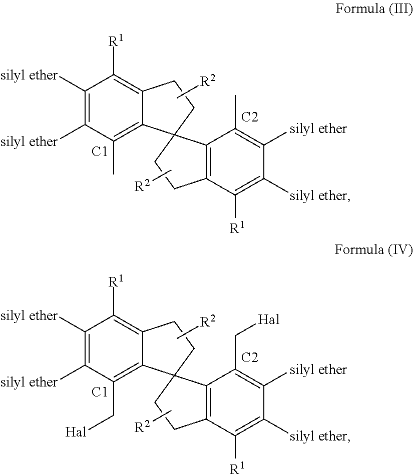

wherein, the silyl ether protecting groups are the same or different, and
wherein, R¹ and R² are as defined previously for Formula (I), and wherein Hal is any one of bromide, chloride or iodide ions, and
b) (i) dehalogenating the biscatechol monomer of Formula (IV) and forming a covalent, intramolecular lock between C1 and C2 of the protected biscatechol monomer of Formula (IV) with a suitable locking group X; or
(ii) substituting the halide ions in Formula (IV) with hydroxyl groups, followed by cyclised dehydration to form a covalent, intramolecular lock between C1 and C2 of the protected biscatechol monomer of Formula (IV);
to provide a silyl ether protected biscatechol monomer of Formula (V) having a locked bicyclic spiro-carbon:

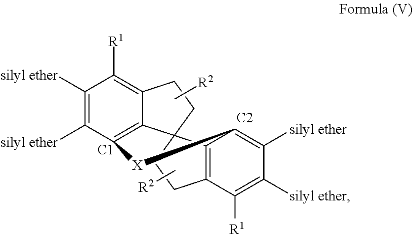

wherein, R¹ and R² and silyl ether are as defined previously for Formula (V), and
(i) X is —CH₂—Y—CH₂—, and wherein Y is O; CH₂; C(═O); C(OR⁷)(OR⁸); NH; NR⁹; S; S(═O); SO₂; C(═S); PH; PR¹⁰; BH; BR¹¹ or BOR¹¹;
   and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or
(ii) X is —CH₂-CH₂—, —CH═CH—, —(═O)O—, —C(═O)NH— or —CHR¹²-CHR¹²—, wherein R¹² are selected from any one or more of H or straight or branched, saturated or unsaturated lower C₁-C₆ alkyl groups, wherein the lower alkyl groups can include aromatic or non-aromatic ring structures.

Preferably, the dehalogenation step (b)(i) to form an intramolecular lock between C1 and C2 is catalysed by a transition metal salt, metal oxide, and/or a pure metal.

Preferably, the transition metal salt is a silver(I), iron(III), titanium(II) or tin(II) salt.

Preferably, the silver(I) salt is AgNO₃ or Ag₂CO₃.

Preferably, the metal oxide is ZnO or Ag₂O.

Preferably, the pure metal is zinc.

Preferably, where X is —CH₂—Y—CH₂— and Y, is O, the dehalogenation step (b)(i) to form an intramolecular lock between C1 and C2 is catalysed by a silver(I) salt, more preferably Ag₂CO₃.

Preferably, the hydroxyl groups in step (b) (ii) undergo cyclised dehydration to form the covalent, intramolecular lock.

Preferably, the silyl ether protecting groups are selected from Formula (VI) as defined in the third aspect.

Preferably, the silyl ether protecting groups are selected from any one or more of trimethylsilyl (TMS), tert-butyldiphenylsilyl (TBDPS), tert-butyldimethylsilyl (TBS) and triisopropylsilyl (TIPS).

Preferably, the silyl ether protecting groups are tert-butyl dimethyl silyl (TBS).

Preferably, each R² is a dimethyl or a C₆ aromatic ring.

Preferably, the biscatechol monomer of Formula (V) contains a fused spiro-bisindane ring system (SBI) or a fused spiro-bisfluorene ring system (SBF).

In a fifth aspect, the present invention provides a silyl ether protected biscatechol monomer of Formula (IV), the biscatechol monomer including a fused spiro-bisindane ring system having a bicyclic spiro-carbon:

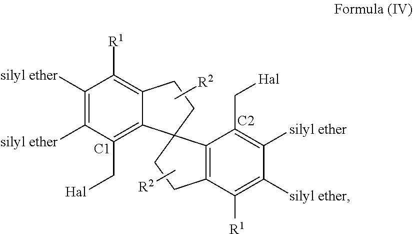

wherein

each R¹ can be the same or different, each R² can be the same or different and wherein R¹ and/or R² are selected from any one or more of H; straight or branched, saturated or unsaturated lower C₁-C₆ alkyl groups, wherein the lower alkyl groups can include aromatic or non-aromatic ring structures R³OR⁴, R³O(C═O)R⁴, R³C(═O)OR⁴, or R³OH, wherein each of R³ and R⁴ are the same or different and are selected from straight or branched, saturated or unsaturated lower C₁-C₆ alkyl groups; and

wherein the silyl ether groups are the same or different.

Preferably, Hal represents any one of bromide, chloride or iodide ions.

Preferably, the silyl ether protecting groups are selected from Formula (VI) as defined in the third aspect.

Preferably, the silyl ether protecting groups are selected from any one or more of trimethylsilyl (TMS), tert-butyldiphenylsilyl (TBDPS), tert-butyldimethylsilyl (TBS) and triisopropylsilyl (TIPS).

Preferably, the silyl ether protecting groups are tert-butyl dimethyl silyl (TBS).

Preferably, each R² is a dimethyl or a C₆ aromatic ring.

Preferably, the biscatechol monomer of Formula (IV) contains a fused spiro-bisindane ring system (SBI) or a fused spiro-bisfluorene ring system (SBF).

In a sixth aspect, the present invention provides a silyl ether protected biscatechol monomer of Formula (V), the biscatechol monomer including a fused spiro-bisindane ring system having a bicyclic spiro-carbon:

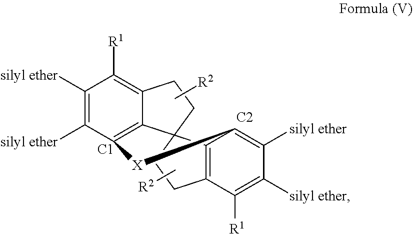

wherein,

each R¹ can be the same or different, each R² can be the same or different and wherein R¹ and/or R² are selected from any one or more of H; straight or branched, saturated or unsaturated lower C₁-C₆ alkyl groups, wherein the lower alkyl groups can include aromatic or non-aromatic ring structures R³OR⁴, R³O(C═O)R⁴, R³C(═O)OR⁴, or R³OH, wherein each of R³ and R⁴ are the same or different and are selected from straight or branched, saturated or unsaturated lower C₁-C₆ alkyl groups; and

wherein,
(a) X is —CH₂—Y—CH₂—, and wherein Y is O; CH₂; C(═O); C(OR⁷)(OR⁸); NH; NR⁹; S; S(═O); SO₂; C(═S); PH; PR¹⁰; BH; BR¹¹ or BOR¹¹;
   and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or
(b) X is —CH₂-CH₂—, —CH═CH—, —(═O)O—, —C(═O)NH— or —CHR¹²-CHR¹²—, wherein R¹² are selected from any one or more of H or straight or branched, saturated or unsaturated lower C₁-C₆ alkyl groups, wherein the lower alkyl groups can include aromatic or non-aromatic ring structures ; and

wherein the silyl ether protecting groups are the same or different.

Preferably, the silyl ether protecting groups are selected from Formula (VI) as defined in the third aspect.

Preferably, the silyl ether protecting groups are selected from any one or more of trimethylsilyl (TMS), tert-butyldiphenylsilyl (TBDPS), tert-butyldimethylsilyl (TBS) and triisopropylsilyl (TIPS).

Preferably, the silyl ether protecting groups are tert-butyl dimethyl silyl (TBS).

Preferably, each R² is a dimethyl or a C₆ aromatic ring.

Preferably, the biscatechol monomer of Formula (V) contains a fused spiro-bisindane ring system (SBI) or a fused spiro-bisfluorene ring system (SBF).

In a seventh aspect, the present invention provides a method of preparing a biscatechol monomer of Formula (VII), the biscatechol monomer including a fused spiro-bisindane ring system having a locked bicyclic spiro-carbon, wherein the method includes the steps of DE protecting a silyl ether protected biscatechol monomer of Formula (V) using a fluoride ion source to form a biscatechol monomer of Formula (VII) having a locked bicyclic carbon:

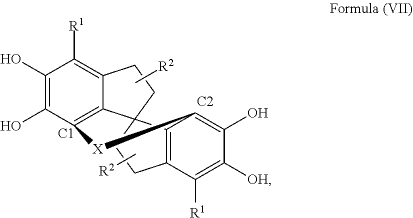

wherein,
(i) X is —CH₂—Y—CH₂—, and wherein Y is O; CH₂; C(═O); C(OR⁷)(OR⁸); NH; NR⁹; S; S(═O); SO₂; C(═S); PH; PR¹⁰; BH; B R¹¹ or BO R¹¹;
   and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or
(ii) X is —CH₂-CH₂—, —CH═CH—, —(═O)O—, —C(═O)NH— or —CHR¹²-CHR¹²—, wherein R¹² are selected from any one or more of H or straight or branched, saturated or unsaturated lower C₁-C₆ alkyl groups, wherein the lower alkyl groups can include aromatic or non-aromatic ring structures.

Preferably, the fluoride ion source is tetrabutylammonium fluoride (TBAF).

Preferably, each R² is a dimethyl or a C₆ aromatic ring.

Preferably, the biscatechol monomer of Formula (VII) contains a fused spiro-bisindane ring system (SBI) or a fused spiro-bisfluorene ring system (SBF).

In an eighth aspect, the present invention provides a biscatechol monomer of Formula (VII), the biscatechol monomer including a fused spiro-bisindane ring system having a locked bicyclic spiro-carbon:

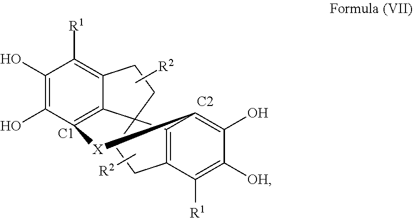

wherein,

each R¹ can be the same or different, each R² can be the same or different and wherein R¹ and/or R² are selected from any one or more of H; straight or branched, saturated or unsaturated lower C₁-C₆ alkyl groups, wherein the lower alkyl groups can include aromatic or non-aromatic ring structures R³OR⁴, R³O(C═O)R⁴, R³C(═O)OR⁴, or R³OH, wherein each of R³ and R⁴ are the same or different and are selected from straight or branched, saturated or unsaturated lower C₁-C₆ alkyl groups; and
(a) X is —CH₂—Y—CH₂—, and wherein Y is O; CH₂; C(═O); C(OR⁷)(OR⁸); NH; NR⁹; S; S(═O); SO₂; C(═S); PH; PR¹⁰; BH; BR¹¹ or BOR¹¹;
   and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or
(b) X is —CH₂-CH₂—, —CH═CH—, —(═O)O—, —C(═O)NH— or —CHR¹²-CHR¹²—, wherein R¹² are selected from any one or more of H or straight or branched, saturated or unsaturated lower C₁-C₆ alkyl groups, wherein the lower alkyl groups can include aromatic or non-aromatic ring structures.

Preferably, each R² is a dimethyl or a C₆ aromatic ring.

Preferably, the biscatechol monomer of Formula (VII) contains a fused spiro-bisindane ring system (SBI) or a fused spiro-bisfluorene ring system (SBF).

In a ninth aspect, the present invention provides the use of a biscatechol monomer of Formula (V) or Formula (VII) in the preparation of a PIM homo- or co-polymer.

Preferably, the preparation of the PIM homo- or co-polymer is via double aromatic nucleophilic substitution in the presence of K₂CO₃.

In a tenth aspect, the present invention provides a fluoride-mediated double nucleophilic aromatic substitution polycondensation (or polymerization) method for the preparation of a PIM polymer.

Preferably, the PIM polymer includes at least one biscatechol monomer of Formula (III), Formula (IV) or Formula (V).

In an eleventh aspect, the present invention provides a fluoride-mediated double nucleophilic aromatic substitution polymerization method for the synthesis of a PIM polymer, wherein the fluoride-mediated polymerization is between a biscatechol monomer and a tetrafluoro aromatic monomer, and wherein the hydroxyl groups on the biscatechol monomer are protected by one or more silyl ether protecting groups.

Preferably, the silyl ether protecting groups are the same.

Preferably, the silyl ether protecting groups are selected from Formula (VI) as defined in the third aspect.

Preferably, the silyl ether protecting groups are selected from any one or more of trimethylsilyl (TMS), tert-butyldiphenylsilyl (TBDPS), tert-butyldimethylsilyl (TBS) and triisopropylsilyl (TIPS).

Preferably, the silyl ether protecting groups are tert-butyl dimethyl silyl (TBS).

Preferably, the tetrafluoro aromatic monomer is 2,3,5,6-tetrafluoroterephthalonitrile.

Preferably, fluoride mediation is provided by organic or inorganic fluoride ion sources.

Preferably, the fluoride ions are sourced from any one or more of potassium fluoride (KF), tetrabutylammonium fluoride (TBAF), cesium fluoride (CsF), organic quaternary ammonium fluoride salts, phosphonium fluoride and other inorganic fluoride salts.

Preferably, sufficient fluoride ions are provided to meet at least a 0.001 molar ratio equivalence of fluoride ions to silyl ether protecting groups present to catalyse the reaction between the biscatechol monomer and the tetrafluoro aromatic monomer.

Preferably, the molar ratio of fluoride ion to silyl ether group is between 0.001 to 4 equivalent.

Preferably, the biscatechol monomer is a biscatechol monomer of Formula (III), Formula (IV) or Formula (V).

In a twelfth aspect, the present invention provides the use of fluoride ions in the manufacture of a PIM polymer.

In a thirteenth aspect, the present invention provides the use of fluoride ions in a fluoride-mediated double nucleophilic aromatic substitution polymerization method for the manufacture of PIM polymers from a biscatechol monomer and a tetrafluoro aromatic monomer, wherein hydroxyl groups on the biscatechol monomer are protected by one or more silyl ether protecting groups.

Preferably, the silyl ether protecting groups are the same.

Preferably, the silyl ether protecting groups are selected from Formula (VI) as defined in the third aspect.

Preferably, the silyl ether protecting groups are selected from any one or more of trimethylsilyl (TMS), tert-butyldiphenylsilyl (TBDPS), tert-butyldimethylsilyl (TBS) and triisopropylsilyl (TIPS).

Preferably, the silyl ether protecting groups are tert-butyl dimethyl silyl (TBS).

Preferably, the tetrafluoro aromatic monomer is 2,3,5,6-tetrafluoroterephthalonitrile.

Preferably, the fluoride ions are provided by organic or inorganic fluoride ion sources.

Preferably, the fluoride ions are sourced from any one or more of potassium fluoride (KF), tetrabutylammonium fluoride (TBAF), cesium fluoride (CsF), organic quaternary ammonium fluoride salts, phosphonium fluoride and other inorganic fluoride salts.

Preferably, sufficient fluoride ions are provided to meet at least a 0.001 molar ratio equivalence of fluoride ions to silyl ether protecting groups present to catalyse the reaction between the biscatechol monomer and the tetrafluoro aromatic monomer.

Preferably, the molar ratio of fluoride ion to silyl ether group is between 0.001 to 4 equivalent.

Preferably, the biscatechol monomer is a biscatechol monomer of Formula (III), Formula (IV) or Formula (V).

In a fourteenth aspect, the invention provides a method for the synthesis of high-molecular weight polymers of intrinsic microporosity (PIMs), the method includes a fluoride-mediated double nucleophilic aromatic substitution polymerization of a biscatechol monomer with a tetrafluoro aromatic monomer, and wherein hydroxyl groups of the biscatechol monomer are protected by one or more silyl ether protecting groups.

Preferably, the silyl ether protecting groups are the same.

Preferably, the silyl ether protecting groups are selected from Formula (VI) as defined in the third aspect.

Preferably, the silyl ether protecting groups are selected from any one or more of trimethylsilyl (TMS), tert-butyldiphenylsilyl (TBDPS), tert-butyldimethylsilyl (TBS) and triisopropylsilyl (TIPS).

Preferably, the silyl ether protecting group is tert-butyl dimethyl silyl (TBS).

Preferably, the tetrafluoro aromatic monomer is 2,3,5,6-tetrafluoroterephthalonitrile.

Preferably, the fluoride ions are provided by organic or inorganic fluoride ion sources.

Preferably, the fluoride ions are sourced from any one or more of potassium fluoride (KF), tetrabutylammonium fluoride (TBAF), cesium fluoride (CsF), organic quaternary ammonium fluoride salts, phosphonium fluoride and other inorganic fluoride salts.

Preferably, sufficient fluoride ions are provided to meet at least a 0.001 molar ratio equivalence of fluoride ions to silyl ether protecting groups present to catalyse the reaction between the biscatechol monomer and the tetrafluoro aromatic monomer.

Preferably, the molar ratio of fluoride ion to silyl ether group is between 0.001 to 4 equivalent.

In a fifteenth aspect, the present invention provides a PIM polymer made by a method or use according to any of the ninth, tenth, eleventh or twelfth aspects of the invention.

A PIM polymer according to the fifteenth aspect of the invention, having a polydispersity of between about 1.5 to about 4, more preferably between about 1.5 to 2.5.

In a sixteenth aspect, the present invention provides a method for preparing a PIM homo-polymer wherein the method includes the step of reacting the biscatechol monomer of Formula (V) or Formula (VII) with a suitable linking monomer.

Preferably, the suitable linking monomer is a tetrahalo aromatic monomer.

Preferably, the tetrahalo aromatic monomer is a tetrafluoro, tetrabromo, tetrachloro or tetraiodo aromatic monomer.

Preferably, the tetrahalo aromatic monomer is a tetrafluoro aromatic monomer.

Preferably, the biscatechol monomer of Formula (V) or Formula (VII) is reacted with a suitable linking monomer in the presence of an organic base, an inorganic base and/or fluoride ions.

Preferably, the biscatechol monomer is of Formula (V) and is base sensitive and the reaction is in the presence of fluoride ions.

Preferably, the fluoride ions are sourced from organic or inorganic fluoride salts.

Preferably, the organic fluoride salts are selected from any one or more of tetrabutylammonium fluoride (TBAF) and other organic quaternary ammonium fluoride salts.

Preferably, the inorganic fluoride salts are selected from any one or more of potassium fluoride (KF), cesium fluoride (CsF) and other inorganic fluoride salts.

Preferably, the biscatechol monomer is of Formula (V) or Formula (VII) is not base sensitive and the reaction is in the presence of an organic or inorganic base.

Preferably, the inorganic base is a carbonate salt.

Preferably, the carbonate salt is K₂CO₃.

Preferably, the organic base is a non-nucleophilic organic base.

Preferably, the non-nucleophilic organic base contains nitrogen.

Preferably, the nitrogen containing organic base is triethylamine.

In a seventeenth aspect, the present invention provides a method of preparing a PIM co-polymer, wherein the method includes the step of reacting together (i) a first biscatechol monomer of Formula (V) or Formula (VII), (ii) a second biscatechol monomer that is different to the first biscatechol monomer, and (iii) a suitable linking monomer.

Preferably, the second biscatechol monomer is a biscatechol monomer of Formula (V) or Formula (VII).

Preferably, the suitable linking monomer is a tetrahalo aromatic monomer.

Preferably, the tetrahalo aromatic monomer is a tetrafluoro, tetrabromo, tetrachloro or tetraiodo aromatic monomer.

Preferably, the tetrahalo aromatic monomer is a tetrafluoro aromatic monomer.

Preferably, the first biscatechol monomer, and the second biscatechol monomer, and the suitable linking monomer are reacted together in the presence of an organic base, an inorganic base and/or fluoride ions.

Preferably, the first biscatechol monomer is of Formula (V) and the first and second biscatechol monomer are base sensitive and the reaction is in the presence of fluoride ions.

Preferably, the fluoride ions are sourced from organic or inorganic fluoride salts.

Preferably, the organic fluoride salts are selected from any one or more of tetrabutylammonium fluoride (TBAF) and other organic quaternary ammonium fluoride salts.

Preferably, the inorganic fluoride salts are selected from any one or more of potassium fluoride (KF), cesium fluoride (CsF) and other inorganic fluoride salts.

Preferably, the first biscatechol monomer of Formula (V) or Formula (VII) is not base sensitive and the reaction is in the presence of an organic or inorganic base.

Preferably, the inorganic base is a carbonate salt.

Preferably, the carbonate salt is K₂CO₃.

Preferably, the organic base is a non-nucleophilic organic base.

Preferably, the non-nucleophilic organic base contains nitrogen.

Preferably, the nitrogen containing organic base is triethylamine.

In an eighteenth aspect, the present invention provides a method of preparing a PIM co-polymer, wherein the method includes the step of reacting together (i) a biscatechol monomer of Formula (V) or Formula (VII), (ii) a first suitable linking monomer, and (iii) a second suitable linking monomer that is different to the first suitable linking monomer.

Preferably, the first and second suitable linking monomers are tetrahalo aromatic monomers.

Preferably, the first and second tetrahalo aromatic monomers are selected from tetrafluoro, tetrabromo, tetrachloro or tetraiodide aromatic monomers.

Preferably, the biscatechol monomer of Formula (V) or Formula (VII) is reacted with the first and second linking monomers in the presence of an organic base, an inorganic base and/or fluoride ions.

Preferably, the biscatechol monomer is of Formula (V) and is base sensitive and the reaction is in the presence of fluoride ions.

Preferably, the fluoride ions are sourced from organic or inorganic fluoride salts.

Preferably, the organic fluoride salts are selected from any one or more of tetrabutylammonium fluoride (TBAF) and other organic quaternary ammonium fluoride salts.

Preferably, the inorganic fluoride salts are selected from any one or more of potassium fluoride (KF), cesium fluoride (CsF) and other inorganic fluoride salts.

Preferably, the biscatechol monomer of Formula (V) or Formula (VII) is not base sensitive and the reaction is in the presence of an organic or inorganic base.

Preferably, the inorganic base is a carbonate salt.

Preferably, the carbonate salt is K₂CO₃.

Preferably, the organic base is a non-nucleophilic organic base.

Preferably, the non-nucleophilic organic base contains nitrogen.

Preferably, the nitrogen containing organic base is triethylamine.

In a nineteenth aspect, the present invention provides a PIM homo- or co-polymer including at least one biscatechol monomer of Formula (VIII):

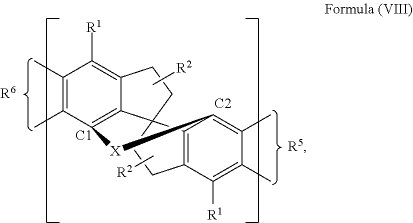

wherein

each R¹ can be the same or different, each R² can be the same or different and wherein R¹ and/or R² are selected from any one or more of H; straight or branched, saturated or unsaturated lower C₁-C₆ alkyl groups, wherein the lower alkyl groups can include aromatic or non-aromatic ring structures R³OR⁴, R³O(C═O)R⁴, R³C(═O)OR⁴, or R³OH, wherein each of R³ and R⁴ are the same or different and are selected from straight or branched, saturated or unsaturated lower C₁-C₆ alkyl groups; and

R⁵ and R⁶ represent suitable linking monomers, wherein the linking monomers can be the same or different and are preferably selected from any one or more tetrahalo aromatic monomers;
(a) X is —CH₂—Y—CH₂—, and wherein Y is O; CH₂; C(═O); C(OR⁷)(OR⁸); NH; NR⁹; S; S(═O); SO₂; C(═S); PH; PR¹⁰; BH; BR¹¹ or BOR¹¹;
   and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or
(b) X is —CH₂-CH₂—, —CH═CH—, —(═O)O—, —C(═O)NH— or —CHR¹²-CHR¹²—, wherein R¹² are selected from any one or more of H or straight or branched, saturated or unsaturated lower C₁-C₆ alkyl groups, wherein the lower alkyl groups can include aromatic or non-aromatic ring structures.

Preferably, each R² is a dimethyl or a C₆ aromatic ring.

Preferably, the biscatechol monomer of Formula (VIII) contains a fused spiro-bisindane ring system (SBI) or a fused spiro-bisfluorene ring system (SBF).

In a twentieth aspect, the present invention provides the use of a PIM homo- or co-polymer including at least one biscatechol monomer of Formula (V), (VII) or Formula (VIII) in the manufacture of gas adsorption, gas purification, gas separation membrane materials, organic sorbent materials, organic dye adsorption and/or organic solvent nanofiltration membrane materials.

### BRIEF DESCRIPTION OF THE DRAWINGS

Embodiments of the invention will now be discussed by way of example only and with reference to the accompanying drawings in which:

FIG. 1 is a schematic diagram showing two known methods for the formation of PIM polymers. In each method, a dibenzodioxin-forming polymerisation is carried out, in which a biscatechol monomer and an activated tetra halo-substituted aromatic are employed to give a polybenzodioxin via double aromatic nucleophilic substitution.

FIG. 2 shows the formation of PIM-1 from the polymerization of a bis-catechol monomer 5,5′,6,6′-tetrahydroxy-3,3,3′,3′-tetramethyl-1,1′-spirobisindane (1) with 2,3,5,6-tetrafluoroterephthalonitrile (2).

FIG. 3 shows a fused spiro-bisindane (SBI) ring system.

FIG. 4 shows ethanoanthracene (EA), Troger's Base (TB), triptycene (TP) and spiro-bisfluorene (SBF) building blocks which have been employed to increase backbone rigidity of PIM polymer chains.

FIG. 5 shows schematic drawings of 7- and 8-membered rings formed by covalently linking C1 and C2 of a core fused spiro-bisindane (SBI) ring system to form an intramolecular lock.

FIG. 6 shows a 3-D molecular model of a biscatechol monomer including a core fused bicyclic spiro-carbon. Hydrogen atoms are omitted for convenience of observation, and the benzyl carbons are labelled C3 and C4. The dashed line indicates the distance between C3 and C4 is about 0.38 nm.

FIG. 7 shows a fused spiro-bisindane (SBI) ring system locked by the formation of an 8-membered ring where X is oxygen.

FIG. 8 shows a locked versus an unlocked core fused spiro-bisindane (SBI) ring system.

FIG. 9 shows suitable silyl ether protecting groups.

FIG. 10 shows examples of known PIM homo- or co-polymers which include a core fused SBI ring system having an unlocked bicyclic spiro-carbon.

FIG. 11 shows a schematic of PIM polymer synthesis via fluoride-mediated polymerisation.

FIG. 12 shows the use of a biscatechol monomer having a locked bicyclic spiro-carbon in the formation of a homo-polymer via carbonate mediated and fluoride mediated polymerisation methods.

FIG. 13 shows the reaction between a representative biscatechol monomer having a locked bicyclic spiro-carbon and a 2,3,5,6-tetrafluoropthalonitrile to give a locked PIM-1 polymer (X═—CH₂—O—CH₂).

FIG. 14 shows examples of suitable tetrafluoro monomers for use in the formation of PIM polymers.

FIG. 15 shows the synthetic route of a TBS-protected biscatechol monomer 2.

FIG. 16 shows the formation of a copolymer from a biscatechol monomer containing a locked bicyclic spiro-carbon, and a second biscatechol monomer linked together by a tetrafluoro aromatic monomer.

FIG. 17 shows the formation of a copolymer from a biscatechol monomer containing a locked bicyclic spiro-carbon, and two different tetrafluoro aromatic monomers.

FIG. 18 shows examples of suitable second biscatechol monomers for use in the formation of PIM co-polymers.

FIG. 19 shows the representative synthetic route for obtaining both silyl ether protected and unprotected biscatechol monomers having a core fused SBI ring system with a locked bicyclic carbon (X═—CH₂—O—CH₂).

FIG. 20 shows the FT-IR spectrum of monomer K4.

FIG. 21 shows the ¹H-NMR spectrum of monomer K4.

FIG. 22 shows HR-MS data of monomer K4.

FIG. 23 shows the raw single crystal structure of monomer K4.

FIG. 24 shows an enlarged view of the ether linkage showing the C—O bond length and C—O—C bond angle of monomer K4.

FIG. 25 shows an enlarged view of the ether linkage showing the dihedral angle between the two benzene planes around the bicyclic spiro-carbon of monomer K4.

FIG. 26 shows ¹H NMR spectrum of deprotected monomer K5.

FIG. 27 shows HR-MS data of deprotected monomer K5.

FIG. 28 shows the representative synthetic scheme for obtaining PIM polymer UOAPIM using fluoride-mediated polymerisation.

FIG. 29 shows the structural comparison of PIM-1 and UOAPIM having the locked bicyclic spiro-carbon in the polymer backbone.

FIG. 30 shows comparison of FT-IR spectra between polymer UOAPIM and PIM-1.

FIG. 31 shows comparison of ¹H NMR spectra between polymer UOAPIM and PIM-1.

FIG. 32 shows an optically clear isotropic film of polymer UOAPIM.

FIG. 33 shows Robeson upper bound plots for CO₂/CH₄, gas pairs showing comparison of polymer UOAPIM (labelled as 1) versus other known PIM polymers for gas separation performance.

FIG. 34 shows Robeson upper bound plots for CO₂/N₂ gas pairs showing comparison of polymer UOAPIM (labelled as 1) versus other known PIM polymers for gas separation performance.

FIG. 35 shows Robeson upper bound plots for O₂/N₂ gas pairs showing comparison of polymer UOAPIM (labelled as 1) versus other known PIM polymers for gas separation performance.

FIG. 36 shows Robeson upper bound plots for H₂/N₂ gas pairs showing comparison of polymer UOAPIM (labelled as 1) versus other known PIM polymers for gas separation performance.

FIG. 37 shows ¹H NMR of biscatechol 1.

FIG. 38 shows ¹³C NMR of biscatechol 1.

FIG. 39 shows ¹H-¹³C HSQC NMR of biscatechol 1.

FIG. 40 shows ¹H-¹³C HMBC NMR of biscatechol 1.

FIG. 41 shows ¹H NMR of TBS-protected biscatechol monomer 2.

FIG. 42 shows ¹³C NMR of TBS-protected biscatechol monomer 2.

FIG. 43 shows ¹H NMR of polymer 4.

FIG. 44 shows GPC traces for fluoride-mediated PIM polymerization under various conditions.

FIG. 45 shows comparison of ¹H NMR spectra of polymer 4 obtaining from a) the original K₂CO₃ mediated synthetic method for the preparation of PIM polymers; and b) the new fluoride-mediated synthetic method for the preparation of PIM polymers.

FIG. 46 shows dihedral potential energy surfaces calculated by using B3LYP functional with the 3-21G+ basis set in Gaussian 09 and their corresponding dihedral rigidity values for different PIM moieties taken from the spring constant of the harmonic model fitting.

FIG. 47 shows molecular dynamics simulations of two oligomers with six repeat units. FIG. 47(a) shows the end-to-end distance, which is plotted in time of the locked spiro-bisindane (upper line) and the unlocked SBI (lower line). FIG. 47(b) shows the distribution in the accessible end-to-end distance for the locked and unlocked spiro-bisindane. Insets of the oligomer conformations at different end-to-end distances are shown.

### DESCRIPTION OF THE INVENTION (BEST MODE)

The present invention provides a method of increasing the rigidity of the structural backbone of high molecular weight polymers of intrinsic microporosity (PIMs or PIM polymers). In particular, the present invention provides a method of increasing the rigidity of PIM homo- or co-polymers which include repeating units containing at least one biscatechol monomer having a core fused spiro-bisindane (SBI) ring system of Formula (I), the method including the step of introducing an intramolecular lock between C1 and C2:

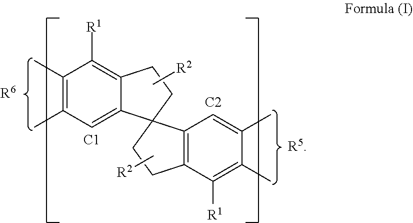

The present invention also provides a fluoride-mediated polymerisation method for the synthesis of high-molecular weight PIM polymers.

High-molecular weight polymers of intrinsic microporosity are herein defined as PIM polymers of intrinsic microporosity having an average molecular weight (Mₙ) of between about 30,000 to about 150,000 Daltons. The preferred molecular weight (Mₙ) is between about 100,000 to about 150,000 Daltons. PIMs produced according to the methods of the invention will preferably have a polydispersity index (M_{w}/Mₙ) of between about 1.5 to about 4. More preferably the polydispersity index will be between about 1.5 and about 2.5.

For the purposes of this specification and claims, an intramolecular lock may be defined as being a covalent bond between the C1 and C2 positions of a traditional fused SBI ring system (see Formula (I)). Reference herein to biscatechol monomers having a fused SBI ring system, or a locked bicyclic spiro-carbon, or locked monomers are intended to refer to the presence of an intramolecular lock between the C1 and C2 positions. For the avoidance of doubt, reference to unlocked or non-locked monomers or an unlocked bicyclic spiro-carbon means an intramolecular lock between the C1 and C2 positions is not present.

Structural Requirements of Biscatechol Monomer Having an Intramolecular Lock

As will be readily understood by a person skilled in the art, a number of suitable substituent groups may be used as shown in Formula (I). However, both R¹ and R² can be the same or different and typically represent H or lower C₁-C₆ alkyl groups which may be straight or branched, saturated or unsaturated, and may include aromatic or non-aromatic ring structures. R¹ and R² may also represent lower C₁-C₆ alkyl ether, ester or alcohol substituents. R⁵ and R⁶ of Formula (I) can be the same or different and represent suitable linking monomers for the formation of PIM polymers. Suitable linking monomers include tetrahalo aromatic monomers and are discussed in more detail below.

The substituents of Formula (I) can therefore be conveniently defined as follows: R¹ and/or R² is any one or more of H; straight or branched, saturated or unsaturated lower C₁-C₆ alkyl groups, wherein the lower alkyl groups can include aromatic or non-aromatic ring structures, R³OR⁴, R³O(C═O)R⁴, R³C(═O)OR⁴, or R³OH, wherein each of R³ and R⁴ are the same or different and are selected from straight or branched, saturated or unsaturated lower C₁-C₆ alkyl groups; and

R⁵ and R⁶ represent suitable linking monomers, wherein the linking monomers can be the same or different and are preferably selected from any one or more tetrahalo aromatic monomers.

As will be apparent to a skilled person, R² in Formula (I) (as well as in Formulae (II), (IIa), (III), (IV), (V), (VII) and (VIII) referred to later herein) includes one or more optional substituents at any one or more of the four positions available on the non-aromatic five membered carbon rings. This can include ring structures formed by two or three methylene units (i.e. —CH2-CH2— or —CH2-CH2-CH2—) between any two of the available positions from the four possible positions on the two non-aromatic five membered carbon rings. Reference can be made to Ten Hoeve and Wynberg¹⁴ (the disclosure of which is referred to and is to be incorporated herein) and in particular to Scheme III for methods of synthesis of such structures.

A preferred option is where each R² is dimethyl (as shown below) and the biscatechol monomer of Formula (I) is:

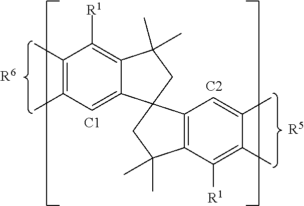

In a particularly preferred option, where each R¹ is H and each R² is dimethyl (as shown above), the biscatechol monomer of Formula (I) contains a fused spiro-bisindane ring system (SBI).

A further preferred option is where each R² is a C₆ aromatic ring (as shown below) and the biscatechol monomer of Formula (I) is:

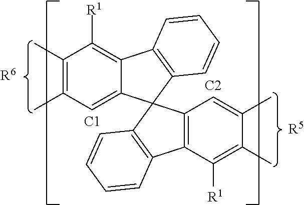

In a particularly preferred option, where each R¹ is H and each R² is a C₆ aromatic ring (as shown above), the biscatechol monomer of Formula (I) contains a fused spiro-bisfluorene ring system (SBF).

As will be apparent to a skilled person, the SBF ring system includes an SBI ring system.

As is clear from Formula (I) and FIG. 3, the fused SBI ring system includes a bicyclic spiro-carbon, which serves as a “pivot point” for some movement. Consequently, use of the traditional fused SBI unit in PIM polymer synthesis results in a relatively flexible backbone structure of the polymer chain. This allows for relative ease of movement of individual chains, even when packed together to form a microporous structure, such that individual pores can fluctuate in size.

However, it is advantageous for PIM polymers to have a rigid backbone structure which hinders efficient space-packing of individual chains to promote a large amount of inter-chain free volume and to reduce pore size variability. This is particularly useful when the PIM polymers are used in gas separation technology.

The inventors have found that it is possible to covalently link C1 and C2 of the traditional fused SBI ring system (see Formula (I)) to form an intramolecular lock (FIG. 5). As a result, movement about the bicyclic spiro-carbon of the SBI ring system is restricted. Thus, the spatial configuration of the bicyclic spiro-carbon is effectively locked, improving the rigidity of the system (see Example Eight). This gives rise to PIM polymers having a rigid backbone structure and, amongst other characteristics, an enhanced gas separation performance.

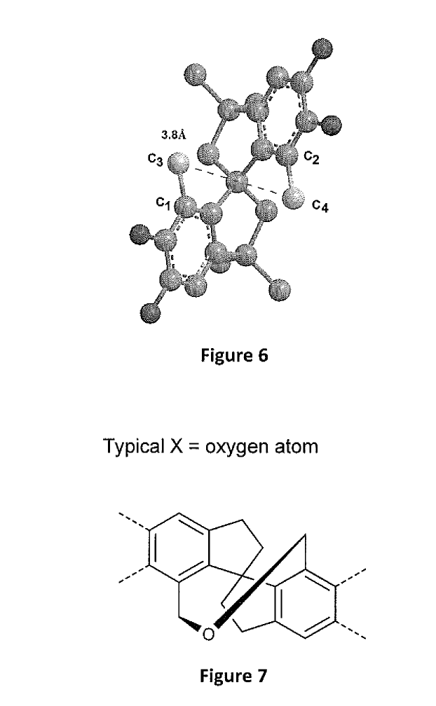

Molecular modelling of Formula (I) calculated the distance between C3 and C4 (see FIG. 6) to be only 3.8 angstrom (Å). This confirmed the possibility of structural connection between C3 and C4 (and hence between C1 and C2) via the introduction of new covalent bonds. According to known bond lengths between carbon atoms or carbon/hetero atoms, two or three atoms were deemed necessary to bridge between C1 and C2 of Formula (I) to form a covalent, intramolecular lock in the form of 7- or 8-membered rings, respectively (FIG. 5).

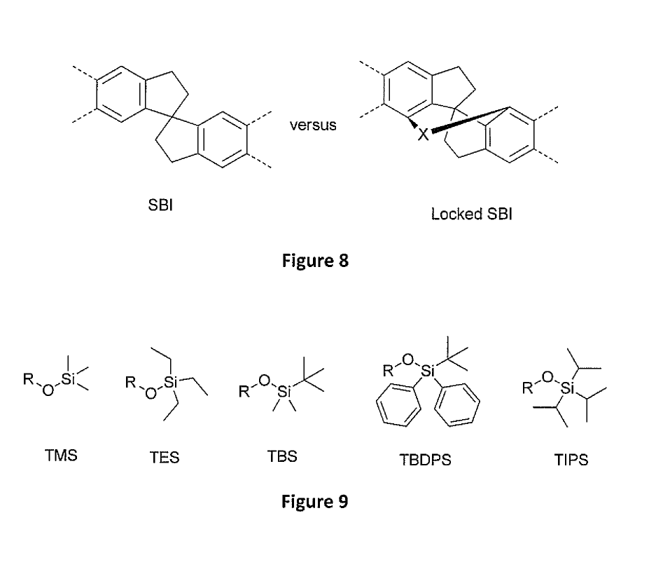

The intramolecular lock between C1 and C2 can be formed from a variety of linking group options that would be known to a person skilled in this art once in possession of this invention. This includes, for an 8 membered ring between C1 and C2: —CH₂—Y—CH₂—, wherein Y is O (FIG. 7); CH₂; C(═O); C(OR⁷)(OR⁸); NH; NR⁹; S; S(═O); SO₂; C(═S); PH; PR¹⁰; BH; BR¹¹ or BOR¹¹; and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; and for a 7 membered ring between C1 and C2: —CH₂-CH₂—, —CH═CH—, —(═O)O—, —C(═O)NH— or —CHR¹²-CHR¹²—, wherein R¹²are selected from any one or more of H or straight or branched, saturated or unsaturated lower C₁-C₆ alkyl groups, wherein the lower alkyl groups can include aromatic or non-aromatic ring structures. FIG. 8 schematically shows a locked versus an unlocked core fused spiro-bisindane (SBI) ring system.

Formation of Biscatechol Monomers with Intramolecular Lock

Formation of a biscatechol monomer having a fused SBI ring system with a locked bicyclic spiro-carbon (i.e. a covalent, intramolecular lock), may be carried out as follows. First, a dimethyl substituted biscatechol monomer containing a fused SBI ring system of Formula (II) is obtained or prepared following known protocols.¹⁵ Each hydroxyl group of the dimethyl substituted biscatechol monomer is then protected by a silyl ether protecting group to give a protected dimethyl substituted biscatechol monomer of Formula (III).

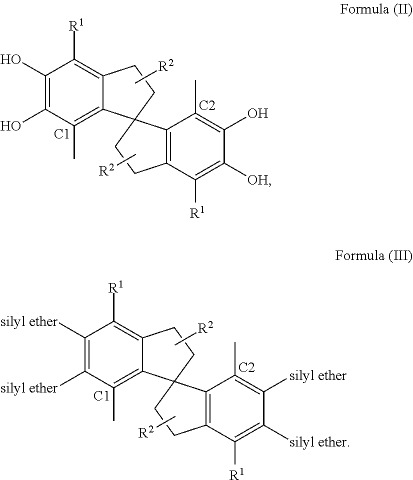

The substituents R¹ and R² are as defined previously for Formula (I) and the silyl ether protecting groups may be the same or different. Silyl ether protecting groups have the general structure R¹³R¹⁴R¹⁵Si—O—R¹⁶(Formula (VI)), in which R¹³, R¹⁴, R¹⁵ and R¹⁶ represent C₁, C₂, C₃ and C₄ alkyl or aryl groups. Examples of silyl ether protecting groups are shown in FIG. 9. Common and readily available silyl ether protecting groups include trimethylsilyl (TMS), tert-butyldiphenylsilyl (TBDPS), tert-butyldimethylsilyl (TBS) and triisopropylsilyl (TIPS). However, a person skilled in the art would understand that a number of other silyl ether protecting groups could be employed for the purpose of protecting hydroxyl groups of biscatechol monomers.

As stated previously in relation to the definition of R² and as will be apparent to a person skilled in the art, R² can include one or more optional substituents at any one or more of the four positions available on the non-aromatic five membered carbon rings. This can include ring structures formed by two or three methylene units (i.e. —CH₂-CH₂— or —CH₂-CH₂-CH₂—) between any two or more of the available positions from the four possible positions on the two non-aromatic five membered carbon rings. As previously noted, reference can be made to Scheme III of Ten Hoeve and Wynberg¹⁴ for methods of synthesis of such structures.

The protected dimethyl substituted biscatechol monomer of Formula (III) is then halogenated at the two methylated positions at C1 and C2 using any one of bromide, chloride or iodide ions (each represented by Hal) to form a silyl ether protected bis(halomethyl) substituted biscatechol monomer of Formula (IV):

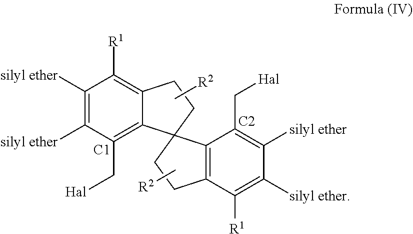

The biscatechol monomer of Formula (V) (see below), including a covalent, intramolecular lock between C1 and C2, can be formed by dehalogenating the biscatechol monomer of Formula (IV) and the inclusion of a suitable linking group indicated as X in Formula (V) below in which the substituents are as defined previously for Formula (I). This is preferably catalysed using a transition metal salt, a metal oxide and/or a pure metal. The transition metal salt is preferably selected from a silver(I), iron(III), Ti(II) or Sn(II) salt, more preferably, AgNO₃ or Ag₂CO₃. Use of a transition metal salt, particularly Ag₂CO₃, is preferred where X is —CH₂—Y—CH₂—and Y is O. The metal oxide is preferably selected from zinc oxide or silver oxide and the pure metal is preferably zinc.

Alternatively, and particularly preferred as an option when Y, is O, the halide ions in Formula (IV) are each substituted by a hydroxyl group via a suitable base (including, but not limited to, sodium hydroxide for example) catalysed hydrolysis method as would be known to a person skilled in the art. The hydroxyl groups then undergo cyclised dehydration to form a biscatechol monomer of Formula (V):

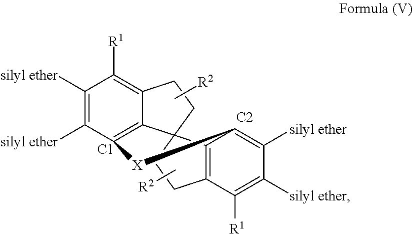

wherein, R¹, R², silyl ether, and X are as defined previously.

As noted above, where the suitable linking group X includes only two linking atoms, a 7-membered ring is formed (FIG. 5). Where the suitable linking group X includes three linking atoms, an 8-membered ring is formed (FIG. 5).

As an alternative, the present invention provides a method of preparing an unprotected biscatechol monomer of Formula (VII), in which the method includes the steps of:
a) halogenating a dimethyl substituted biscatechol monomer of Formula (II) having a bicyclic spiro-carbon to form a bis(halomethyl) substituted biscatechol monomer of Formula (IIa):

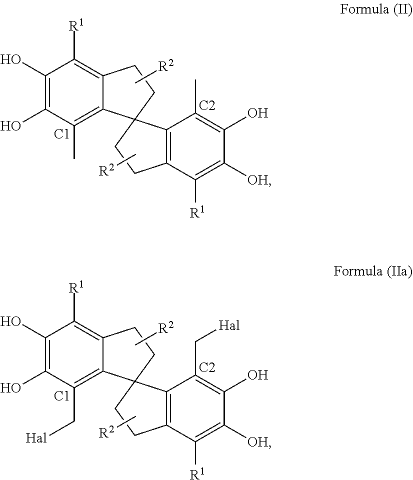

wherein, R¹, R² and Hal are as defined previously for Formula (IV);
b) (i) dehalogenating the biscatechol monomer of Formula (IIa) and forming a covalent, intramolecular lock between C1 and C2 of the biscatechol monomer of Formula (IIa) with a suitable locking group X; or
   (ii) substituting the halide ions in Formula (IIa) with hydroxyl groups, followed by cyclised dehydration to form a covalent, intramolecular lock between C1 and C2 of the biscatechol monomer of Formula (IIa);
   to provide a biscatechol monomer of Formula (VII) having a locked bicyclic spiro-carbon:

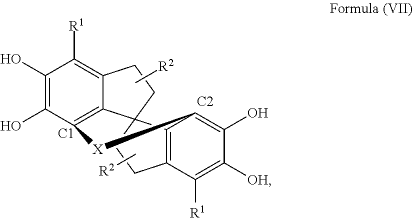

wherein, R¹, R² and X are as defined previously for Formula (V).

Again, as will be apparent to a person skilled in the art, R² and as will be apparent to a person skilled in the art, R² can include one or more optional substituents at any one or more of the four positions available on the non-aromatic five membered carbon rings. This can include ring structures formed by two or three methylene units (i.e. —CH₂-CH₂— or —CH₂-CH₂-CH₂—) between any two or more of the available positions from the four possible positions on the two non-aromatic five membered carbon rings. As previously noted, reference can be made to Scheme III of Ten Hoeve and Wynberg¹⁴ for methods of synthesis of such structures.

In a further alternative, the unprotected biscatechol monomer of Formula (VII) may be prepared by deprotecting a silyl ether protected biscatechol monomer of Formula (V) using a fluoride ion source such as tetrabutylammonium fluoride (TBAF). This alternative method is described in detail in Example One below.

Thus, the present invention also provides a locked silyl ether protected biscatechol monomer of Formula (V) and a locked unprotected biscatechol monomer of Formula (VII).

Formation of PIM Polymers

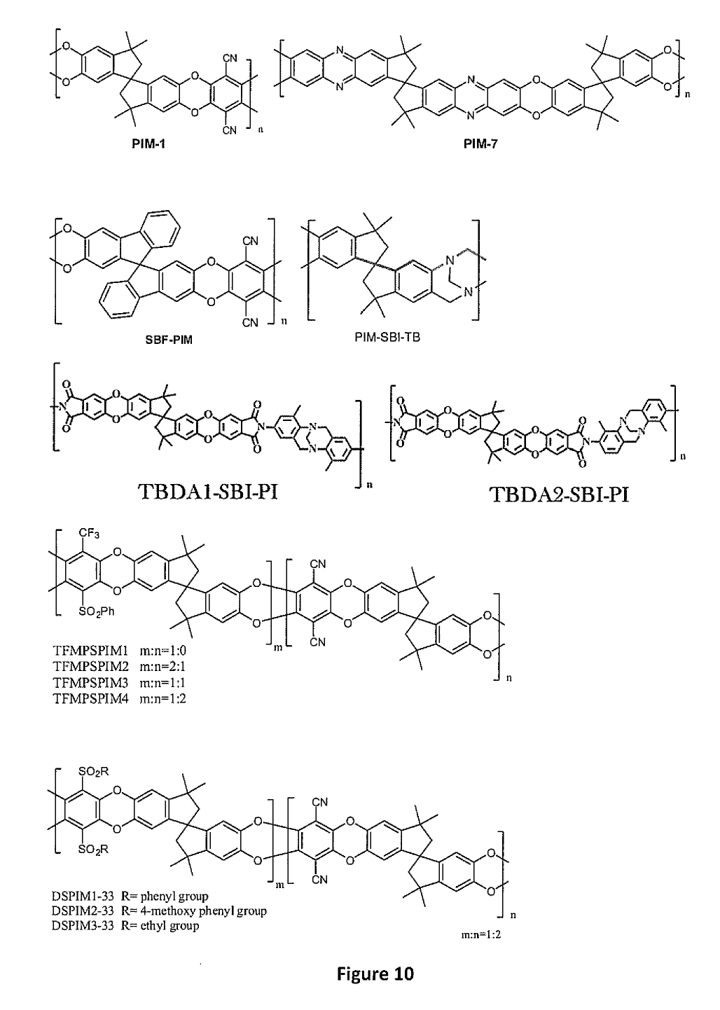

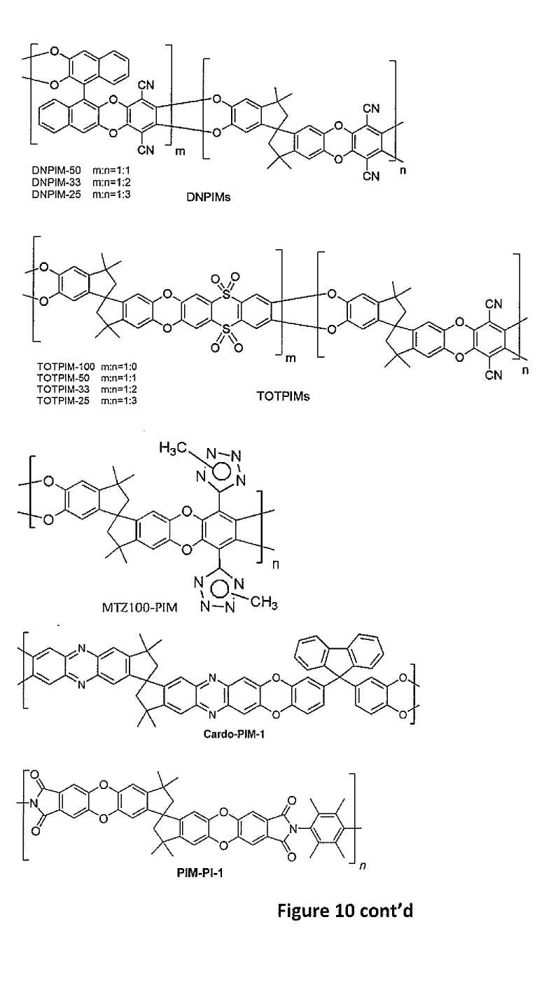

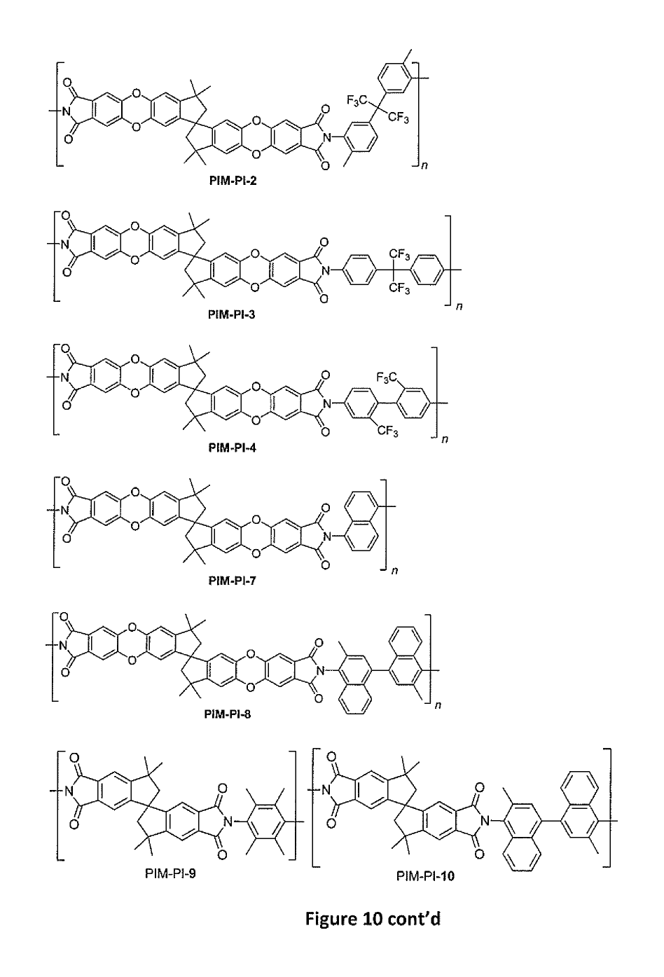

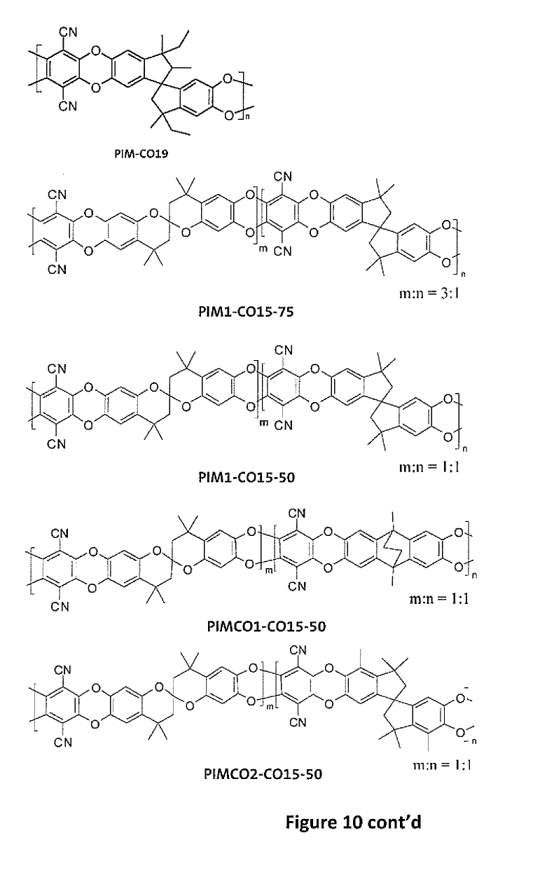

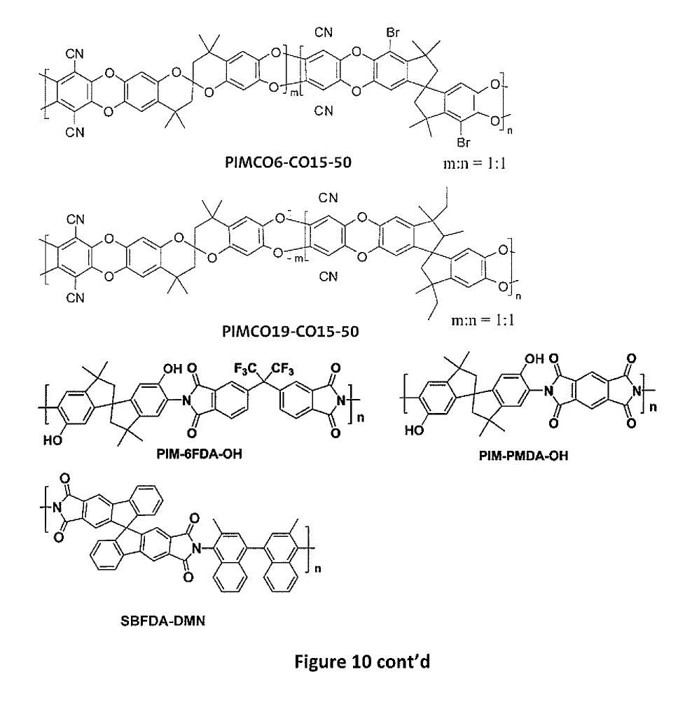

Either of the locked monomers of Formulae (V) or (VII) can be readily used in the formation of PIM homo- and co-polymers (discussed in detail below). A number of PIM polymers which include biscatechol monomers having a fused SBI ring system with an unlocked bicyclic spiro-carbon are shown in FIG. 10 by way of example. As would be apparent to a skilled person, FIG. 10 shows such biscatechol monomers together with suitable linking monomers as would be used in PIM polymers. The biscatechol monomers in FIG. 10 could be replaced in PIM polymers with the same biscatechol monomers but including a locked bicyclic spiro-carbon according to Formula (V) and (VII) of the present invention. The locked versions of the unlocked monomers (shown in FIG. 10) to be used in the preparation of PIM polymers can be prepared according the methods described herein. Biscatechol monomers, and PIM polymers including such biscatechol monomers together with linking monomers, as shown in FIG. 10 but including an intramolecular lock formed by a linking group between C1 and C2 over the bicyclic spiro-carbon, form part of the present invention.

Further biscatechol monomers having an unlocked bicyclic spiro-carbon, and PIM polymers including those biscatechol monomers together with linking monomers, are disclosed in U.S. Pat. No. 7,690,514 B2 to Budd and McKeown, the disclosure of which is hereby specifically incorporated by way of reference. Preparing PIM polymers following the preparation methods disclosed in U.S. Pat. No. 7,690,514 B2 using a biscatechol monomer containing a locked bicyclic spiro-carbon according to the present invention (Formula (V) or Formula (VII)) in place of a biscatechol monomer containing an unlocked bicyclic spiro-carbon, as are shown in U.S. Pat. No. 7,690,514 B2 (or as shown in FIG. 10), will result in a PIM polymer of increased rigidity in comparison to those shown in U.S. Pat. No. 7,690,514 B2 (or FIG. 10). Biscatechol monomers, and PIM polymers including such biscatechol monomers together with linking monomers, as shown in U.S. Pat. No. 7,690,514 B2 including an intramolecular lock formed by a linking group between C1 and C2 over the bicyclic spiro-carbon form part of the present invention.

Thus, the present invention provides the use of biscatechol monomers having a fused SBI ring system with a locked bicyclic spiro-carbon (Formula (V) or Formula (VII)) in the preparation of PIM homo- or co-polymers. Reference to PIM polymers generally herein is intended to include both homo- and co-polymers, unless the context clearly indicates otherwise. Also provided are methods for the preparation of PIM homo- and co-polymers including such monomers and PIM homo- or co-polymers including at least one biscatechol monomer of Formula (V) or Formula (VII).

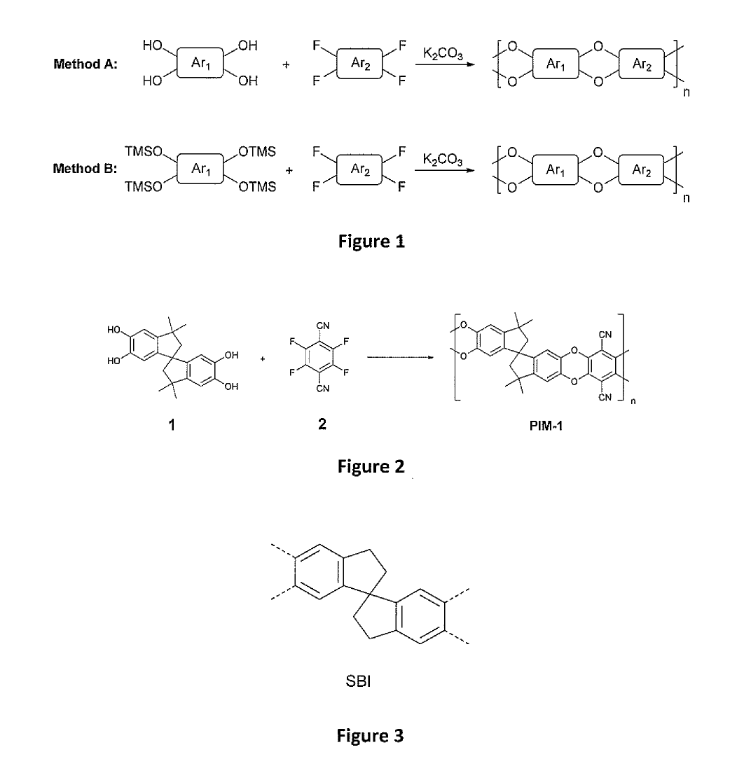

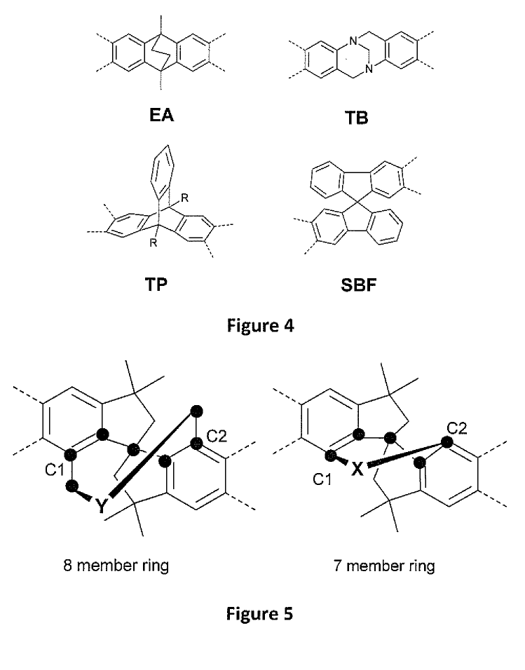

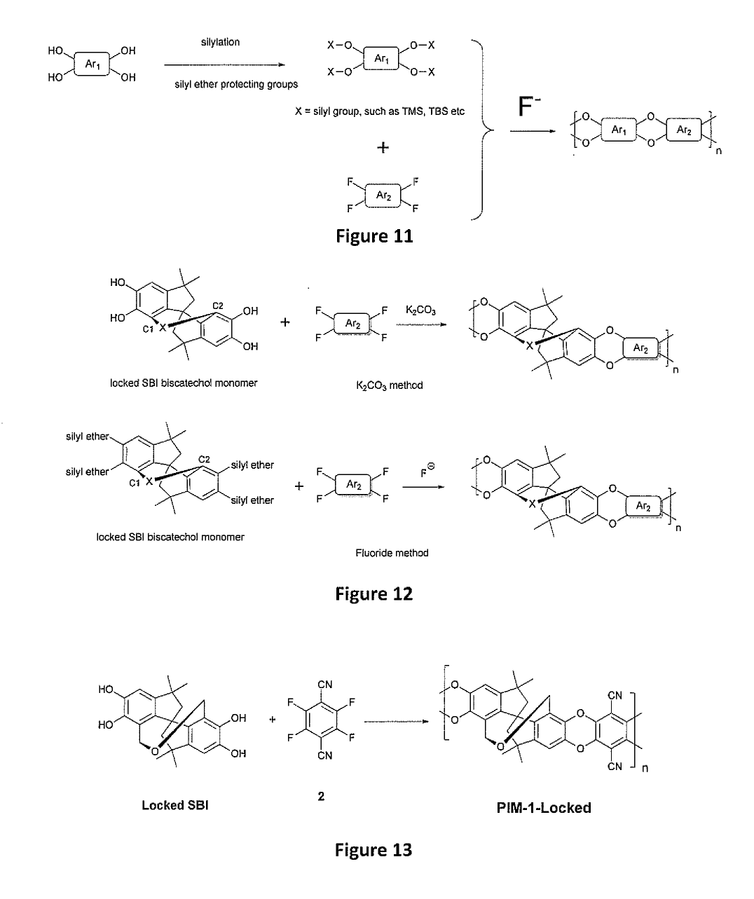

FIG. 1 shows two existing double nucleophilic aromatic substitution methods for the formation of PIM polymers. Methods A and B are comparative method examples, both requiring the presence of K₂CO₃ to catalyze the reaction. Surprisingly, the inventors have found that fluoride-mediated double nucleophilic aromatic substitution polycondensation (or polymerization) (FIG. 11) successfully results in the formation of high quality high-molecular weight PIM polymers.¹⁶

Formation of PIM Homo-Polymers

Preparation of a PIM homo-polymer includes the step of reacting a biscatechol monomer of Formula (V) or Formula (VII) with a suitable linking monomer (e.g. R⁵/R⁶from Formula (I)) (FIGS. 12 and 13).

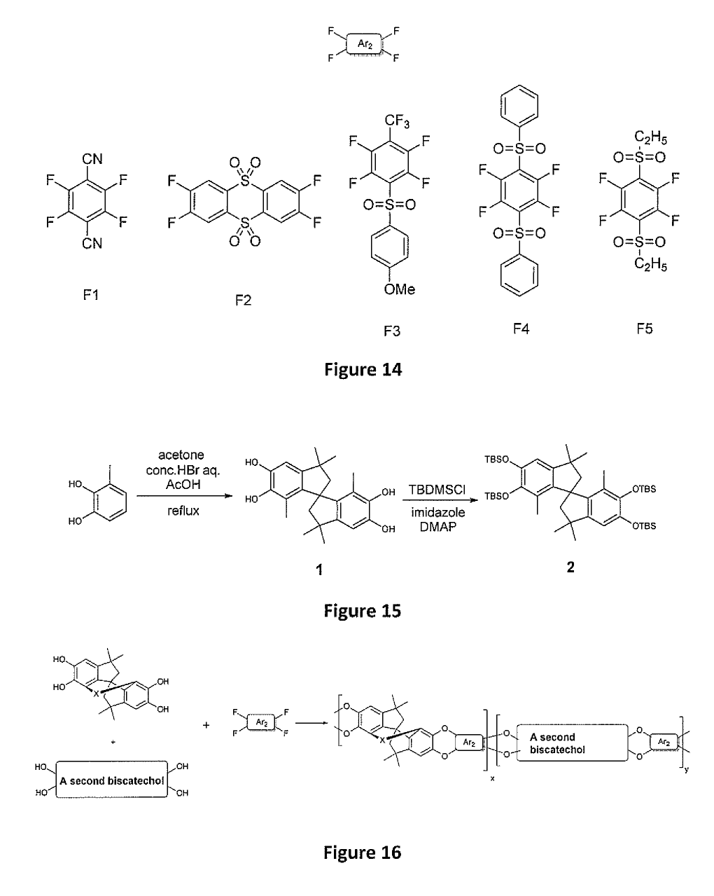

Any suitable linking monomer (R⁵/R⁶) can be used, as would be known to a person skilled in the art. Preferably, suitable linking monomers are chosen from tetrahalo aromatic monomers. Examples of tetrahalo aromatic monomers include tetrafluoro, tetrabromo, tetrachloro and tetraiodide aromatic monomers. Examples of tetrafluoro aromatic monomers are shown in FIG. 14. Further tetrahalo aromatic monomer options are disclosed in U.S. Pat. No. 7,690,514 B2 to Budd and McKeown to which the reader is again referred and the contents of which is hereby specifically incorporated by way of reference.

The biscatechol monomer of Formula (V) or Formula (VII) will be reacted with the suitable linking monomer in the presence of an organic base, an inorganic base and/or a fluoride ion source. The choice of preferred base depends on the base sensitivity of the biscatechol monomer.

Polymerisation with Base Sensitive Monomers—Fluoride-Mediated Polymerisation

Where the biscatechol monomer of Formula (V) or Formula (VII) is base sensitive and therefore chemically unstable in the presence of K₂CO₃, then the reaction is preferably conducted in the presence of the fluoride ions. Examples of base sensitive functional groups include amide groups, aldehyde groups, ester groups, benzyl ether groups, halogenoalkanes and acidic groups such as carboxylic acids. Fluoride mediated polymerisation is particularly suited where mild reaction conditions are required. However, where fluoride mediated polymerisation is employed, the hydroxyl groups of the monomer must first be protected (e.g. by silyl ether protecting groups, Formula (V)) to facilitate polymerisation.

The fluoride ions can be sourced from any suitable organic or inorganic fluoride ion sources as would be known to those skilled in the art. Common examples of organic and inorganic fluoride ion sources include potassium fluoride (KF), tetrabutylammonium fluoride (TBAF), cesium fluoride (CsF), organic quaternary ammonium fluoride salts, phosphonium fluoride and other inorganic fluoride salts, for example. Of these, KF, TBAF and CsF are readily available. TBAF and organic quaternary ammonium fluoride salts have the advantage that they allow for the formation of “metal-free” PIM polymers. Preferably, the organic fluoride salts are selected from any one or more of TBAF and other organic quaternary ammonium fluoride salts. Preferably, the inorganic fluoride salts are selected from any one or more of KF, CsF and other inorganic fluoride salts.

As would be understood by the skilled person, a sufficient amount of fluoride ions must be present to catalyse the polymerization reaction between a biscatechol monomer and a tetrafluoro aromatic monomer. Preferably, the minimum, catalytic amount of fluoride ions required is a 0.001 molar ratio equivalence of fluoride ions to silyl ether protecting groups present. Any amount above the 0.001 molar ratio equivalence will provide the necessary catalysis. Preferably, the molar ratio of fluoride ion to silyl ether group is between about 0.001 to 2 or 3 or 4 equivalent.

With reference to FIG. 11, as a first step in the preparation of a PIM polymer using fluoride-mediated polymerization, hydroxyl groups of a biscatechol monomer must be protected. Silyl ether protecting groups are particularly suitable for use in fluoride-mediated polymerization, particularly TMS, TBDPS, TBS and TIPS as they can be installed and removed very selectively under mild conditions by fluoride ions in solution.

The inventors have found that the TBS group is a particularly good protecting group where fluoride-mediated polymerization is to be employed. The TBS protection group possesses greater stability under various reaction conditions, for example, the hydrolytic stability of TBS silyl ether is ca. 10⁴ times more than that of TMS protecting group.¹⁷ TBS derivatives crystallize easily,¹⁸ and multiple purification techniques (e.g. recrystallization, column chromatography, aqueous extraction) are accessible for TBS-protected monomers.

The inventors hypothesize that the fluoride ions cleave the silyl ether bonds of the protected biscatechol monomer to restore the phenoxide anions, thus allowing polymerization between the tetrafluoro aromatic monomer and the biscatechol monomer. Any tetrafluoro aromatic monomer can be employed in the synthesis of a PIM polymer and examples of suitable tetrafluoro aromatic monomers are shown in, but not limited to, FIG. 14. The inventors have found 2,3,5,6-tetrafluoroterephthalonitrile to be particularly useful where fluoride-mediated polymerization is employed.

Polymerisation with Non-Base Sensitive Monomers

Where the biscatechol monomer of Formula (V) or Formula (VII) is not base sensitive, organic or inorganic bases can also be employed in the polymerisation process. Fluoride-mediated polymerisation is also a method, as is discussed below. Suitable organic bases are any non-nucleophilic organic base including, but not limited to, nitrogen containing organic bases such as triethylamine. Suitable inorganic bases include, but are not limited to, carbonate salts such as K₂CO₃. A preferred polymerisation reaction is as described in U.S. Pat. No. 7,690,514 B2 to which the reader is again referred and the contents of which is hereby specifically incorporated by way of reference. This preferred polymerisation reaction uses K₂CO₃ as the base, however other options as described in U.S. Pat. No. 7,690,514 B2 can also be used.

The fluoride-mediated polymerization method can also be used with non-base sensitive biscatechol monomers, provided the hydroxyl groups are protected. Thus the fluoride-mediated polymerization method has broad application to both non-base sensitive and base sensitive monomers, and also to the preparation of PIM polymers including locked and unlocked monomers. Fluoride-mediated polymerization is applicable to any biscatechol structure. For illustration purposes, the inventors used bis-catechol monomer 5,5′,6,6′-tetrahydroxy-3,3,3′,3′-tetramethyl-1,1′-spirobisindane 1. A typical synthetic method for the preparation of biscatechol 1 is shown in FIG. 15. Reaction of biscatechol 1 monomer with tert-butyl dimethyl silyl (TBS) chloride gives a TBS-protected biscatechol monomer 2 (FIG. 15).

The TBS-protected biscatechol monomer is then polymerized with a tetrafluoro aromatic monomer 3 in the presence of fluoride ions sourced from KF, TBAF and CsF. This yields PIM polymer 4 (see synthetic route in Table 4 below).

Formation of PIM Co-Polymers

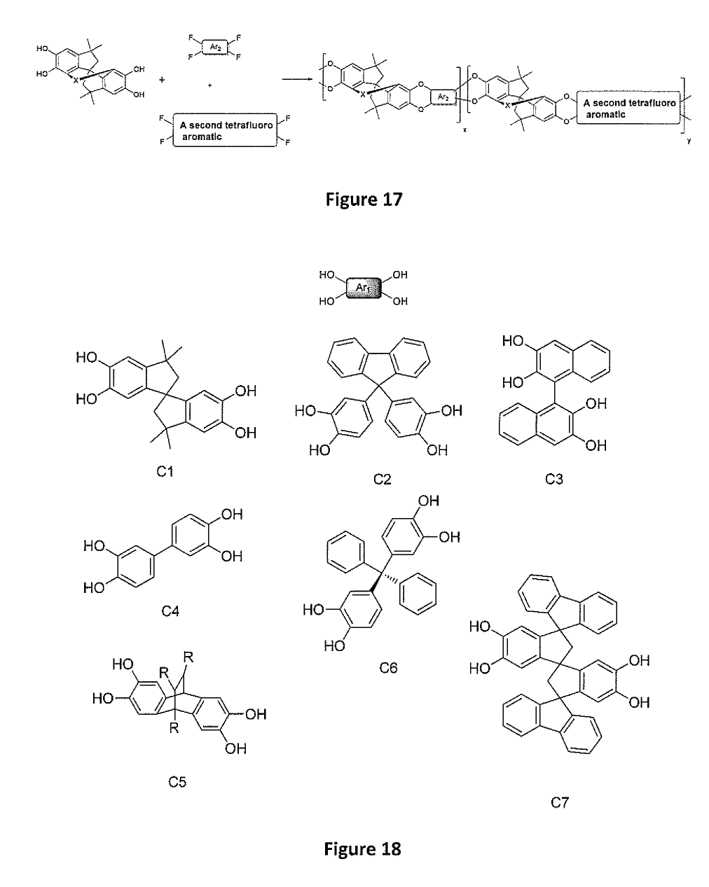

Preparation of a PIM Co-Polymer can be Achieved by Either:
a) reacting a first biscatechol monomer of Formula (V) or Formula (VII) with a second, different biscatechol monomer and a suitable linking monomer in the presence of an organic base, an inorganic base and/or a fluoride ion source (FIG. 16); or
b) reacting a biscatechol monomer of Formula (V) or Formula (VII) with first and second suitable linking monomers in the presence of an organic base, an inorganic base and/or a fluoride ion source (FIG. 17).

Suitable linking monomers, organic and inorganic bases and fluoride ion sources are as defined above. As will be understood by a person skilled in the art, PIM co-polymers incorporating two or more different types of biscatechol monomers linked together by two or more different types of linking monomers can also be prepared according to the general method of the present invention. Thus, the present invention also provides a PIM homo- or co-polymer including at least one biscatechol monomer of Formula (VIII):

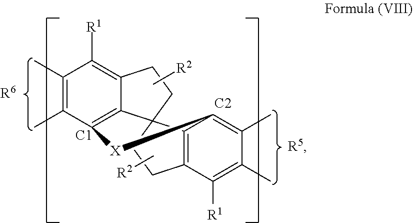

where each of R¹, R², R⁵, R⁶ and X are as previously defined.

With respect to option (a) above for the formation of PIM co-polymers, examples of suitable second biscatechol monomers are shown in FIG. 18. Further biscatechol monomer options are also disclosed in U.S. Pat. No. 7,690,514 B2 to Budd and McKeown to which the reader is again referred and the contents of which is hereby specifically incorporated by way of reference. The second biscatechol monomer may also include a fused SBI ring system having a locked bicyclic spiro-carbon according to the present invention. Suitable linking monomers (R⁵/R⁶) are as noted above.

A wide range of PIM co-polymers can be synthesised by pairing different biscatechol monomers with tetrahalogen aromatic monomers (option (a) and (b) above). There are many advantages of carrying out co-polymerisation as opposed to homo-polymerisation. For example, if one particular PIM homo-polymer is insoluble, the PIM co-polymer may be soluble due to the introduction of a highly soluble second biscatechol PIM monomer. By varying the combination and the feed ratios of PIM monomers, the resulting PIM polymers may have enhanced solubility, gas permeability and selectivity and these features can be tailored to meet different needs. In some cases, a synergistic enhancement of features may be observed.

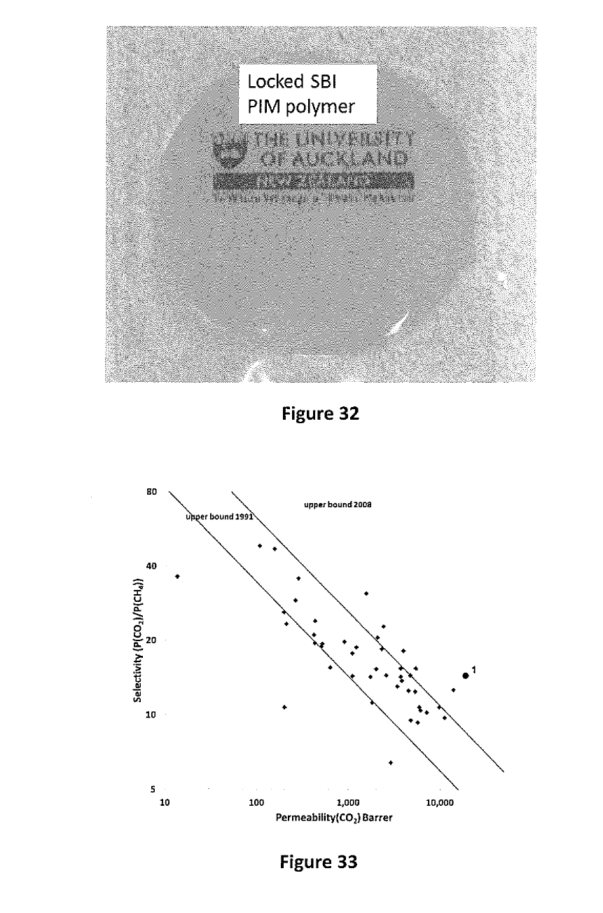

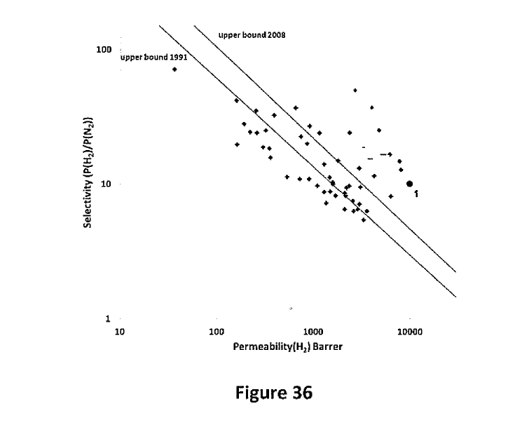

PIM polymers according to the invention have been found to be mechanically robust (as best seen in FIG. 32). Robeson upper bound plots for CO₂/CH₄, CO₂/N₂, O₂/N₂, and H₂/N₂ gas pairs (see FIGS. 33-36) show that PIM polymers according to the invention exhibit excellent characteristics. PIM polymers according to the invention therefore offer excellent alternatives to known options and have potential application in gas adsorption, gas purification, gas separation materials, organic sorbent materials, organic dye adsorption, and/or complex mixtures separation in liquid form such as organic solvent nanofiltration membrane materials.

Formation of PIM Polymers Having Locked Bicyclic Spino-Carbon from Unlocked Precursor Monomers

As will be appreciated by a person skilled in the art, biscatechol monomers of Formula (III) or Formula (IV) could also be utilised in the formation of a PIM polymer incorporating locked bicyclic spiro-carbons. As a first step, biscatechol monomers of Formula (III) or Formula (IV) could be polymerized with a tetrafluoro monomer to form a long chain “precursor” (or “pre-lock”) polymer. The C1 and C2 positions of the biscatechol monomers could then be locked by the formation of an intramolecular lock to give a PIM polymer with locked bicyclic spiro-carbons.

The inventors hypothesize that this approach would not result in all bicyclic spiro-carbons being locked. It is therefore not the preferred route for increasing the rigidity of PIM polymers.

### EXAMPLES

Formation of an intramolecular lock between C1 and C2

### Example One

General Synthetic Route for Obtaining a Biscatechol Monomer Including a Core Fused SBI Ring System with a Locked Bicyclic Spiro-Carbon (K4)

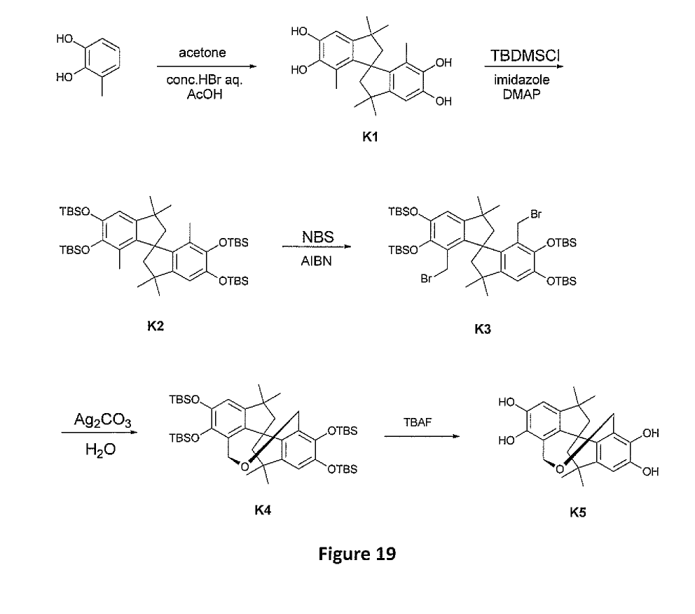

As shown in FIG. 19, the synthesis is carried out in four steps. First, dimethyl substituted biscatechol monomer K1 having a core fused SBI ring system was synthesised via a condensation reaction of 3-methyl catechol and acetone under harsh acidic conditions. The high electron density in the methyl catechol starting material allowed access to nucleophilic addition, and further intramolecular cyclisation was induced by dehydration to form the target dimethyl substituted biscatechol monomer K1. In contrast to normal biscatechol monomers employed for PIM-1 synthesis, in order to lock the bicyclic spiro-carbon, it was necessary to first synthesis K1. Silylation of the four phenolic hydroxyl groups of K1 was then carried out to protect the hydroxyl groups to form silyl ether protected biscatechol monomer K2. Tert-butyl dimethyl silyl chloride (TBDMSC1) was employed as a silylation reagent with imidazole as a base, and the reaction was catalysed by DMAP. The TBS-protected biscatechol monomer K2 was then brominated using N-bromosuccinimide (NBS) via a free-radical benzyl bromination to give dibenzylbromide biscatechol K3. Compounds K1, K2 and K3 were characterised by ¹H, ¹³C NMR. The resulting spectra agree well with the target molecules. The final cyclization step to deliver biscatechol monomer K4 having a locked bicyclic spiro-carbon was successfully achieved by using Ag₂CO₃ under relatively mild, neutral conditions.

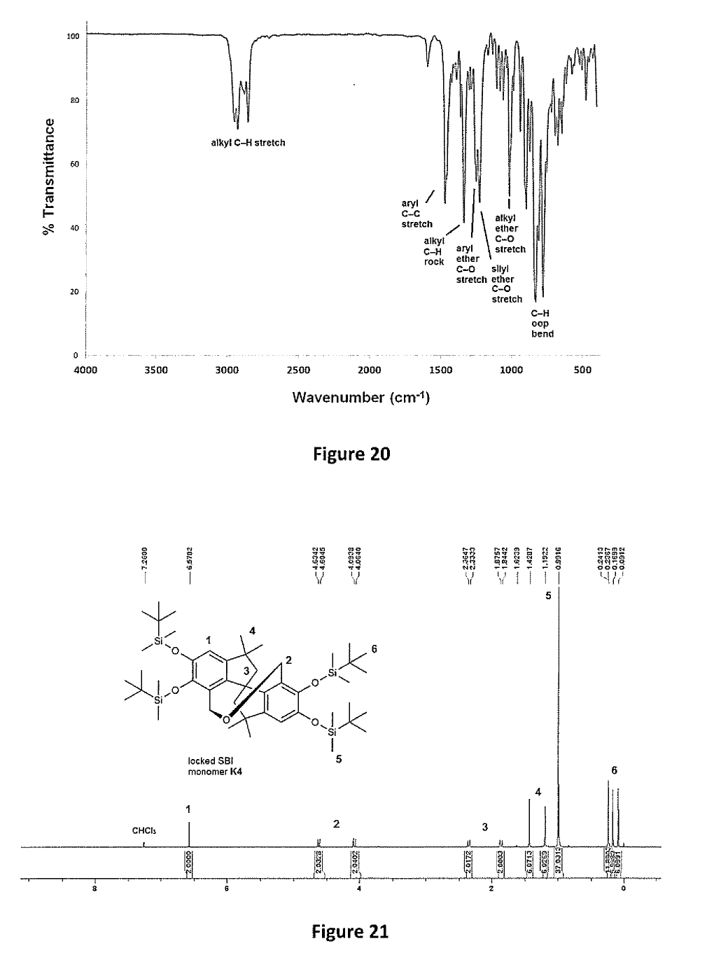

The FT-IR spectrum of silyl ether protected biscatechol monomer K4 (FIG. 20) showed an adsorption band corresponding to the classical C—O—C stretching and bending vibration, confirming that the desired intramolecular lock had been formed.

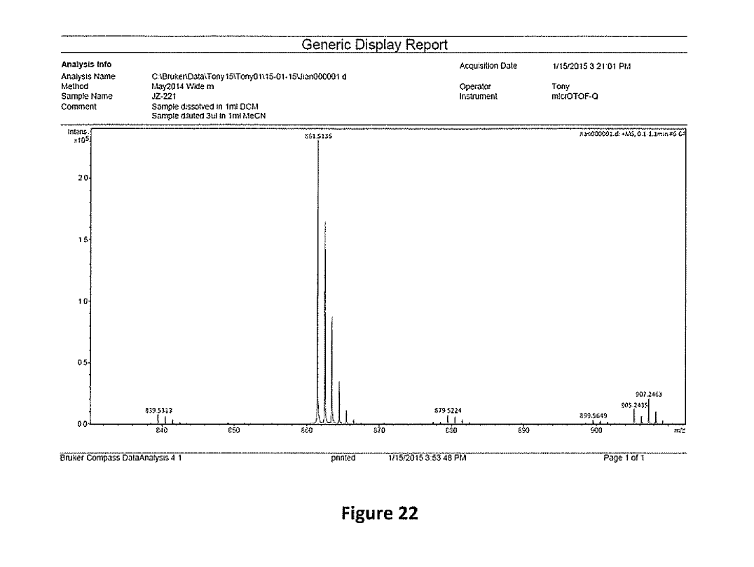

The structure of K4 was further characterised by ¹H NMR (FIG. 21) and HR-MS (FIG. 22). Both agree well with the proposed structure. Interestingly, in the ¹H NMR spectrum, diastereotopic protons were also found in the two methylene groups, giving two doublets at 4.62-4.07 and 2.34-1.85, respectively. HR-MS was carried out and found 861.5136 for the [M+Na]⁺ signal (calcd. 861.5132 for C₄₇H₈₂NaO₅Si₄).

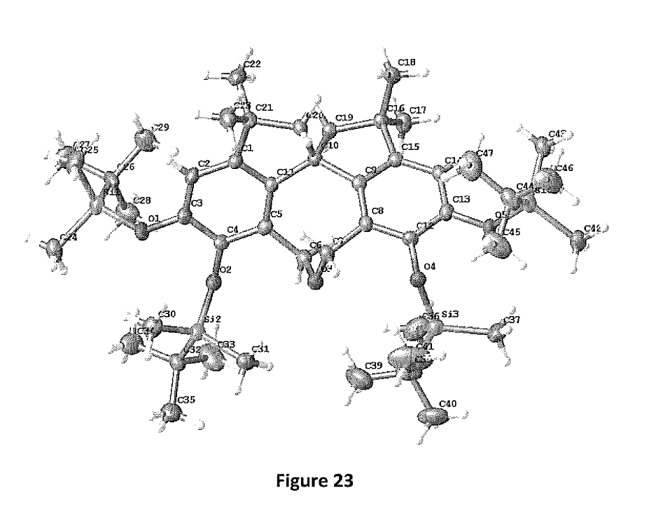

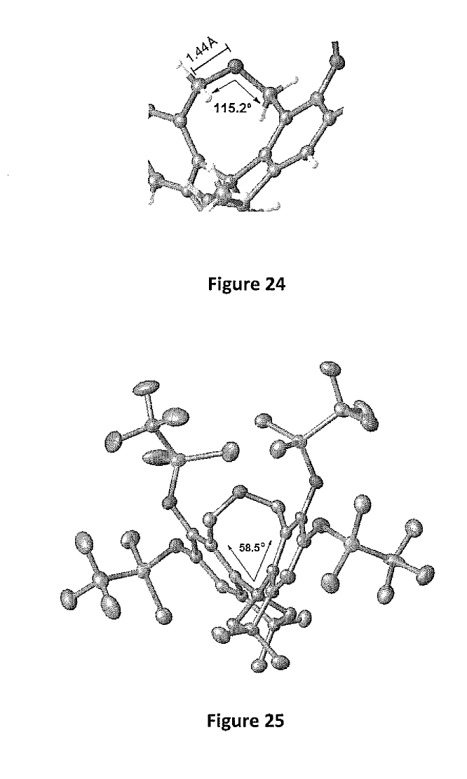

The most convincing evidence of the formation of biscatechol monomer K4 were the results obtained from the single-crystal XRD analysis of K4 (FIG. 23). A tetra-TBS-protected SBI ring system locked by a dibenzyl ether was observed. The mean value of C-O ether bond connecting the two benzyl carbons was found to be 1.44 Å (FIG. 24), which is longer than the average C—O bond length of ethers (1.42 Å). Also, the C—O—C bonds angle of the ether were found to be 115.2° larger than that of common ethers (about)112° (FIG. 24). The dihedral angle between the two benzene planes around the bicyclic spiro-carbon in monomer K4 was 58.5° (FIG. 25), which is significantly smaller than the normal dihedral angle of a SBI ring system (ca.)85°. The single-crystal XRD analysis of K4 therefore clearly shows that due to intramolecular locking, the SBI ring system is more distorted and strained, and hence an enhanced forbidden movement of the bicyclic spiro-carbon is accomplished. Tables 1 and 2 below show bond length and bond angle data for monomer K4.

TABLE 1
Bond lengths for monomer K4

| Atom | Atom | Length/Å |
|---|---|---|
| C1 | C2 | 1.390(4) |
| C1 | C11 | 1.387(4) |
| C1 | C21 | 1.516(4) |
| C2 | C3 | 1.388(4) |
| C3 | C4 | 1.403(4) |
| C3 | O1 | 1.379(3) |
| C4 | C5 | 1.403(4) |
| C4 | O2 | 1.378(3) |
| C5 | C6 | 1.507(4) |
| C5 | C11 | 1.402(4) |
| C6 | O3 | 1.440(3) |
| C7 | C8 | 1.501(4) |
| C7 | O3 | 1.440(3) |
| C8 | C9 | 1.402(4) |
| C8 | C12 | 1.399(4) |
| C9 | C10 | 1.530(3) |
| C9 | C15 | 1.387(4) |
| C10 | C11 | 1.533(4) |
| C10 | C19 | 1.555(4) |
| C10 | C20 | 1.554(4) |
| C12 | C13 | 1.398(4) |
| C12 | O4 | 1.384(3) |
| C13 | C14 | 1.386(4) |
| C13 | O5 | 1.385(3) |
| C14 | C15 | 1.390(4) |
| C15 | C16 | 1.518(4) |
| C16 | C17 | 1.533(4) |
| C16 | C18 | 1.538(4) |
| C16 | C19 | 1.554(4) |
| C20 | C21 | 1.546(4) |
| C21 | C22 | 1.538(4) |
| C21 | C23 | 1.528(4) |
| C24 | Si1 | 1.855(3) |
| C25 | Si1 | 1.855(3) |
| C26 | C27 | 1.599(4) |
| C26 | C28 | 1.545(4) |
| C26 | C29 | 1.550(4) |
| C26 | Si1 | 1.816(4) |
| C30 | Si2 | 1.863(3) |
| C31 | Si2 | 1.857(3) |
| C32 | C33 | 1.531(4) |
| C32 | C34 | 1.539(4) |
| C32 | C35 | 1.536(4) |
| C32 | Si2 | 1.874(3) |
| C36 | Si3 | 1.850(3) |
| C37 | Si3 | 1.852(4) |
| C38 | C39 | 1.537(5) |
| C38 | C40 | 1.534(5) |
| C38 | C41 | 1.539(5) |
| C38 | Si3 | 1.876(3) |
| C42 | Si4 | 1.861(3) |
| C43 | Si4 | 1.861(3) |
| C44 | C45 | 1.536(5) |
| C44 | C46 | 1.547(4) |
| C44 | C47 | 1.546(5) |
| C44 | Si4 | 1.876(3) |
| O1 | Si1 | 1.6579(19) |
| O2 | Si2 | 1.663(2) |
| O4 | Si3 | 1.671(2) |
| O5 | Si4 | 1.6638(19) |

TABLE 2
Bond angles for monomer K4

| Atom | Atom | Atom | Angle/° |
|---|---|---|---|
| C2 | C1 | C21 | 126.7(2) |
| C11 | C1 | C2 | 120.8(3) |
| C11 | C1 | C21 | 112.5(2) |
| C3 | C2 | C1 | 119.7(3) |
| C2 | C3 | C4 | 119.6(2) |
| O1 | C3 | C2 | 122.0(2) |
| O1 | C3 | C4 | 118.4(2) |
| C3 | C4 | C5 | 120.6(2) |
| O2 | C4 | C3 | 119.0(2) |
| O2 | C4 | C5 | 120.3(2) |
| C4 | C5 | C6 | 120.2(2) |
| C11 | C5 | C4 | 118.6(2) |
| C11 | C5 | C6 | 120.6(2) |
| O3 | C6 | C5 | 110.9(2) |
| O3 | C7 | C8 | 111.3(2) |
| C9 | C8 | C7 | 120.6(2) |
| C12 | C8 | C7 | 120.5(2) |
| C12 | C8 | C9 | 118.3(2) |
| C8 | C9 | C10 | 129.1(2) |
| C15 | C9 | C8 | 120.3(2) |
| C15 | C9 | C10 | 110.6(2) |
| C9 | C10 | C11 | 120.8(2) |
| C9 | C10 | C19 | 99.7(2) |
| C9 | C10 | C20 | 112.2(2) |
| C11 | C10 | C19 | 112.7(2) |
| C11 | C10 | C20 | 100.2(2) |
| C20 | C10 | C19 | 111.6(2) |
| C1 | C11 | C5 | 120.1(2) |
| C1 | C11 | C10 | 110.3(2) |
| C5 | C11 | C10 | 129.6(2) |
| C13 | C12 | C8 | 120.8(2) |
| O4 | C12 | C8 | 119.4(2) |
| O4 | C12 | C13 | 119.8(2) |
| C14 | C13 | C12 | 119.8(2) |
| O5 | C13 | C12 | 118.7(2) |
| O5 | C13 | C14 | 121.2(2) |
| C13 | C14 | C15 | 119.4(3) |
| C9 | C15 | C14 | 120.8(2) |
| C9 | C15 | C16 | 112.5(2) |
| C14 | C15 | C16 | 126.7(2) |
| C15 | C16 | C17 | 111.8(2) |
| C15 | C16 | C18 | 111.4(2) |
| C15 | C16 | C19 | 100.6(2) |
| C17 | C16 | C18 | 108.1(2) |
| C17 | C16 | C19 | 110.4(2) |
| C18 | C16 | C19 | 114.4(2) |
| C16 | C19 | C10 | 108.0(2) |
| C21 | C20 | C10 | 107.9(2) |
| C1 | C21 | C20 | 101.1(2) |
| C1 | C21 | C22 | 110.5(2) |
| C1 | C21 | C23 | 112.0(2) |
| C22 | C21 | C20 | 113.6(2) |
| C23 | C21 | C20 | 110.8(2) |
| C23 | C21 | C22 | 108.7(2) |
| C27 | C26 | Si1 | 109.5(2) |
| C28 | C26 | C27 | 104.3(3) |
| C28 | C26 | C29 | 109.4(3) |
| C28 | C26 | Si1 | 113.5(2) |
| C29 | C26 | C27 | 104.9(3) |
| C29 | C26 | Si1 | 114.4(2) |
| C33 | C32 | C34 | 109.4(3) |
| C33 | C32 | C35 | 108.4(3) |
| C33 | C32 | Si2 | 110.1(2) |
| C34 | C32 | Si2 | 110.6(2) |
| C35 | C32 | C34 | 108.2(3) |
| C35 | C32 | Si2 | 110.1(2) |
| C39 | C38 | C41 | 108.0(4) |
| C39 | C38 | Si3 | 110.6(2) |
| C40 | C38 | C39 | 108.6(3) |
| C40 | C38 | C41 | 109.4(3) |
| C40 | C38 | Si3 | 110.1(3) |
| C41 | C38 | Si3 | 110.1(3) |
| C45 | C44 | C46 | 108.5(3) |
| C45 | C44 | C47 | 109.4(3) |
| C45 | C44 | Si4 | 111.2(2) |
| C46 | C44 | Si4 | 108.8(2) |
| C47 | C44 | C46 | 107.7(3) |
| C47 | C44 | Si4 | 111.1(2) |
| C3 | O1 | Si1 | 131.06(18) |
| C4 | O2 | Si2 | 131.15(17) |
| C7 | O3 | C6 | 115.2(2) |
| C12 | O4 | Si3 | 127.97(17) |
| C13 | O5 | Si4 | 127.75(17) |
| C24 | Si1 | C25 | 107.74(15) |
| C26 | Si1 | C24 | 112.90(14) |
| C26 | Si1 | C25 | 111.28(15) |
| O1 | Si1 | C24 | 103.95(13) |
| O1 | Si1 | C25 | 112.41(13) |
| O1 | Si1 | C26 | 108.39(12) |
| C30 | Si2 | C32 | 111.92(14) |
| C31 | Si2 | C30 | 108.68(15) |
| C31 | Si2 | C32 | 110.41(14) |
| O2 | Si2 | C30 | 112.14(13) |
| O2 | Si2 | C31 | 109.80(12) |
| O2 | Si2 | C32 | 103.84(12) |
| C36 | Si3 | C37 | 109.8(2) |
| C36 | Si3 | C38 | 112.19(16) |
| C37 | Si3 | C38 | 109.69(18) |
| O4 | Si3 | C36 | 108.95(13) |
| O4 | Si3 | C37 | 112.07(14) |
| O4 | Si3 | C38 | 104.10(13) |
| C42 | Si4 | C43 | 109.70(14) |
| C42 | Si4 | C44 | 111.25(15) |
| C43 | Si4 | C44 | 110.54(15) |
| O5 | Si4 | C42 | 104.05(13) |
| O5 | Si4 | C43 | 110.32(12) |
| O5 | Si4 | C44 | 110.80(12) |

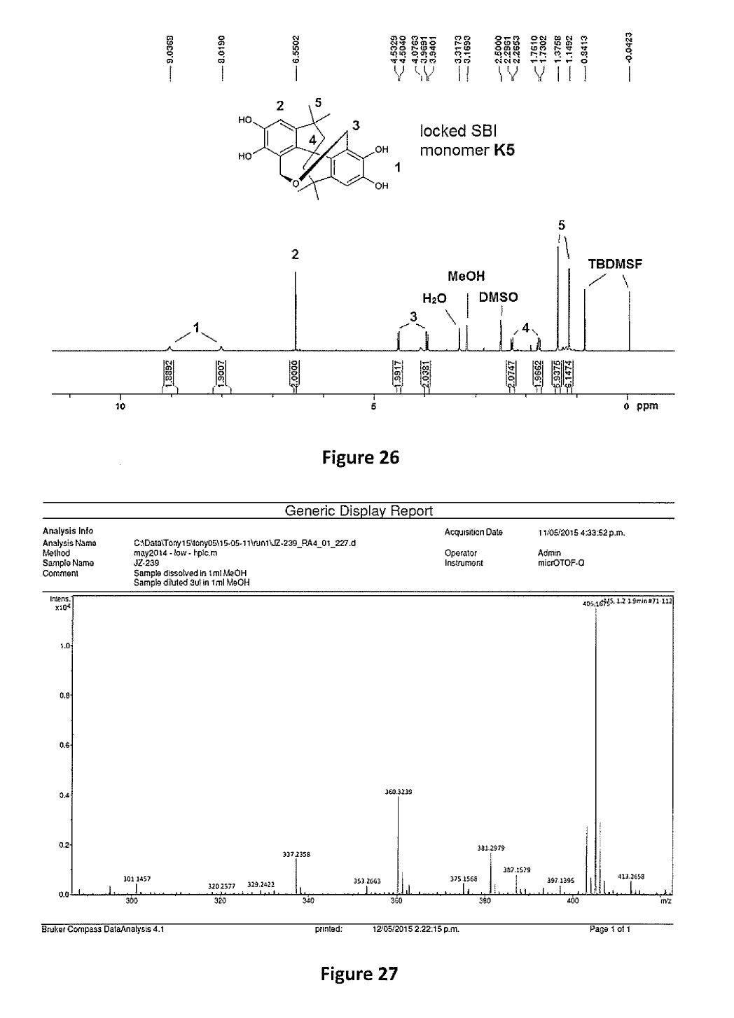

As shown in FIG. 19, monomer K4 may be deprotected by tetrabutylammonium fluoride (TBAF) to give deprotected monomer K5. The structure of K5 was characterised by ¹H NMR (FIG. 26) and HR-MS (FIG. 27).

### Synthesis of 5,5′,6,6′-Tetrahydroxy-3,3,3′,3′,7,7′-hexamethylspirobisindane (K1)

To a mixture of conc. HBr aq. (36 ml) and acetic acid (33 ml) was added 1,2-dihydroxy-3methylbenzene (16.8 g, 135 mmol) to give a clear solution. Acetone (21 ml, 286 mmol, 2.1 eq) was then added drop wisely. After addition, the resulting solution was heated to reflux and kept refluxing for 24 hours. The hot mixture was poured into water (360 ml) with vigorous stirring. The precipitate was filtered out. Then the solid was stirred in acetic acid (150 ml) for 1 hour, filtered, and washed with acetic acid to give title compound K1 (15 g, 40.7 mmol, 60%) as a white solid.

¹H NMR (400 MHz, DMSO-d₆): δ ppm 8.83 (br, 2H), 7.67 (br, 2H), 6.43 (s, 2H), 2.16-2.05 (m, 4H), 1.51 (s, 6H), 1.25 (s, 6H), 1.22 (s, 6H); ¹³C NMR (100 MHz, DMSO-d₆): δ ppm 143.95, 142.32, 141.28, 137.46, 119.76, 106.02, 56.96, 56.67, 41.76, 32.54, 29.87, 10.74; HR-MS (ESI) calcd. 391.1880 for C₂₃H₂₈NaO₄, found 391.1868 [M+Na]⁺.

### Synthesis of 5,5′,6,6′-Tetrakis(tert-butyldimethylsilyloxy)-3,3,3′,3′,7,7′-hexamethylspirobisindane (K2)

To a suspension of the bis-catechol K1 (10.85 g, 29.4 mmol) in anhydrous DMF (135 ml) was added t-butyl dimethyl silyl chloride (36 g, 239 mmol). The mixture was cooled down in ice-bath and added imidazole (24 g, 353 mmol) slowly within 5 minutes followed by addition of DMAP (0.36 g, 2.95 mmol). The mixture was stirred under Ar at room temperature for 3 days. Afterwards, the mixture was poured into methanol (400 ml) slowly and stirred for 1 hour. Then, the precipitate was filtered, washed with methanol and dried under vacuum to give the title compound K2 (22.6 g, 27.4 mmol, 93%) as white solid. ¹H NMR (400 MHz, CDCl₃): δ ppm 6.48 (s, 2H), 2.24-2.17 (m, 4H), 1.62 (s, 6H), 1.29 (s, 6H), 1.27 (s, 6H), 0.97 (s, 18H), 0.96 (s, 18H), 0.22 (s, 6H), 0.19 (s, 6H), 0.14 (s, 6H), 0.05 (s, 6H); ¹³C NMR (100 MHz, CDCl₃): δ ppm 146.21, 144.21, 143.72, 139.96, 125.99, 111.95, 57.90, 56.76, 42.24, 32.69, 29.72, 26.33, 26.27, 18.84, 18.59, 12.73, −3.04, −3.31, −3.68, −3.70; HR-MS (ESI) calcd. 825.5519 for C₄₇H₈₅O₄Si₄, found 825.5540 [M+H]⁺.

### Synthesis of Brominated Monomer K3

Using CCl₄ as solvent: To a solution of dimethyl TTBS monomer K2 (1 g, 1.21 mmol) in CCl₄ (20 ml) was added NBS (0.44 g, 2.47 mmol), followed by addition of AIBN (20 mg, 0.12 mmol). The mixture was brought to reflux for 18 h. Then, the mixture was cooled down to room temperature and poured into methanol (200 ml) to crystalize. The crystals was filtered and washed with methanol to give the title compound K3 (0.93 g, 0.946 mmol, 78%) as slightly yellowish white crystals.

¹H NMR (400 MHz, CDCl₃): δ ppm 6.67 (s, 2H), 4.17 (d, J=9.6 Hz, 2H), 3.86 (d, J=9.6 Hz, 2H), 2.71 (d, J=13.0 Hz, 2H), 2.23 (d, J=13.0 Hz, 2H), 1.40 (s, 6H), 1.29 (s, 6H), 1.01 (s, 18H), 0.95 (s, 18H), 0.30 (s, 6H), 0.28 (s, 6H), 0.19 (s, 6H), −0.02 (s, 6H); ¹³C NMR (100 MHz, CDCl₃): δ ppm 147.26, 145.79, 145.32, 140.76, 126.20, 115.74, 58.64, 55.81, 42.91, 32.78, 30.06, 26.83, 26.54, 26.27, 19.15, 18.76, −2.25, −2.67, −3.65, −4.11; HR-MS (ESI) calcd. 1003.3549 for C₄₇H₈₂Br₂NaO₄Si₄, found 1003.3538 [M+Na]⁺.

### Synthesis of Monomer K4 Having a Locked Bicyclic Carbon

The dibromomethyl TTBS intermediate K3 (3.4 g, 3.46 mmol) and Ag₂CO₃ (4.77 g, 17.3 mmol) were added to a mixture of dioxane (160 ml) and water (16 ml). Then, the slurry was brought to reflux atmosphere with effective stirring for 66 h. The mixture was filtered when hot and the solid washed with dichloromethane. The filtrate was evaporated. The residue was partitioned between water and dichloromethane, and the aqueous phase was extracted by dichloromethane for another time. The organic layers were combined, dried over anhydrous Na₂SO₄ and evaporated to give monomer K4 (3.1 g, 3.7 mmol, quant.) as a lightly yellow dense oil, which was solidified to give crystals upon storage. Monomer 10 was purified by recrystallization from EtOH before use.

¹H NMR (400 MHz, CDCl₃): δ ppm 6.58 (s, 2H), 4.62 (d, J=11.9 Hz, 2H), 4.08 (d, J=11.9 Hz, 2H), 2.35 (d, J=12.6 Hz, 2H), 1.86 (d, J=12.6 Hz, 2H), 1.43 (s, 6H), 1.19 (s, 6H), 0.99 (s, 36H), 0.24 (s, 6H), 0.23 (s, 6H), 0.17 (s, 6H), 0.09 (s, 6H) (FIG. 18); ¹³C NMR (100 MHz, CDCl₃): δ ppm 147.15, 144.08, 143.62, 142.90, 121.70, 114.31, 58.10, 57.85, 56.78, 41.53, 32.39, 30.19, 26.36, 26.25, 18.86, 18.64, −3.34, −3.59, −3.66, −3.98; HR-MS (ESI) calcd. 861.5132 for C₄₇H₈₂NaO₅Si₄, found 861.5136 [M+Na]⁺(FIG. 22).

### Synthesis of Deprotected Monomer K5 Having a Locked Bicyclic Carbon

To a solution of compound K4 (2 g, 2.4 mmol) in THF (20 ml) was added AcOH (0.6 g, 10 mmol), followed by dropwise addition of 1N TBAF solution in THF (10 ml, 10 mmol) at 0° C. The resulting mixture was stirred at 0° C. for 1.5 h. Then, the mixture was evaporated to remove THF, and the residue was dissolved in EtOAc and washed with water 5 times. The organic layer was dried over Na₂SO₄, evaporated and purified by flash silica gel column using DCM and MeOH (10:1) as eluent to give the title compound K5 (0.76 g, 2.0 mmol, 83%) as red dense oil which solidified upon storage.

¹H NMR (400 MHz, DMSO-d₆): δ ppm 9.04 (s, 2H), 8.02 (s, 2H), 6.55 (s, 2H), 4.53-3.94 (m, 4H), 2.30-1.73 (m, 4H), 1.38 (s, 6H), 1.15 (s, 6H) (FIG. 23); HR-MS (ESI) calcd. 405.1672 for C₂₃H₂₆NaO₅, found 405.1675 [M+Na]⁺ (FIG. 27).

### Example Two

General Synthetic Route for Obtaining a PIM Polymer Including Monomer K4 (UOAPIM)

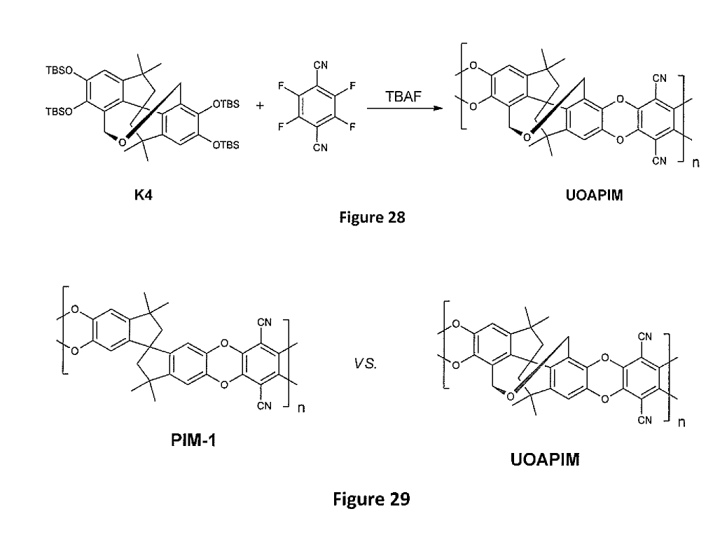

Synthesis of PIM polymer UOAPIM was conducted using TBS-protected monomer K4 and the most common tetrafluoro monomer (2,3,5,6-tetrafluoroterephthalonitrile) according to FIG. 28. Instead of utilising the traditional PIM synthesis method, we found that fluoride-mediated polymerisation is particularly useful in the preparation of UOAPIM as K4 is unstable under traditional polymerisation conditions. The characterisation of the target polymer UOAPIM are completed by FTIR, NMR, BET, GPC. FIG. 29 provides a structural comparison between the well-known PIM-1 polymer and UOAPIM.

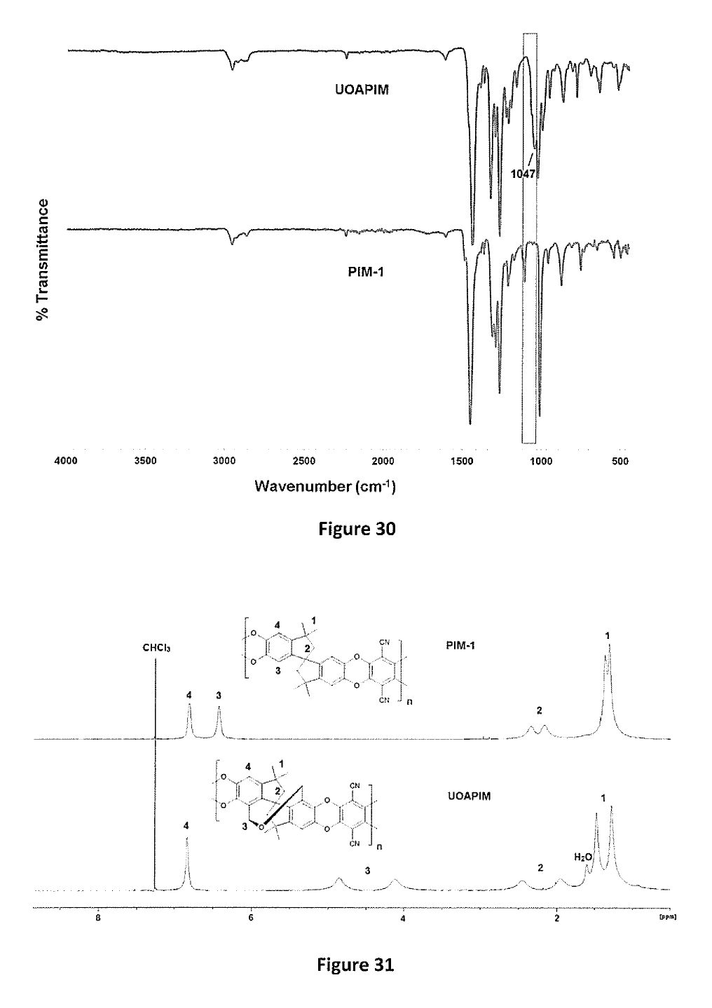

FT-IR spectra of both polymers (UOAPIM and PIM-1) are shown in FIG. 30. Since polymer UOAPIM is an ether derivative of PIM-1, their FT-IR spectra are somewhat similar. An additional adsorption band was observed at 1047cm⁻¹ for polymer UOAPIM, which could be assigned to the aliphatic ether C—O—C vibration band.

¹H NMR spectra are shown in FIG. 31, where all peaks agree with the target structures. Notably, as indicated by NMR, the introduction of the ether linkage results in a remarkable difference between the two polymers PIM-1 and UOAPIM in terms of chemical shifts of the protons labelled 1 in the methyl groups or the protons labelled 2 in the methylene groups.

Detailed Synthesis Procedure of Polymer UOAPIM

To a mixture of monomer K4 (0.6396 g, 0.762 mmol) and 2,3,5,6-tetrafluoro-terephthalonitrile (0.1524 g, 0.762 mmol) was added anhydrous NMP (5.8 ml) to give a suspension. 1N TBAF in THF (0.076 ml, 0.076 mmol) was diluted 10 times with anhydrous NMP. The resulting 0.1N TBAF solution (0.076 ml, 0.0076 mmol) was added to the reaction mixture at room temperature. The reaction mixture was heated to 160° C. gradually within 45 min and kept at 160° C. for 1 h. The mixture was cooled down to 60° C. and diluted with NMP (3 ml). Then, it was poured into EtOAc (60 ml) with vigorous stirring for lh. The solid was filtered and washed with EtOAc to give crude product which was re-dissolved in CHCl₃ and precipitated from MeOH to give polymer UOAPIM (0.35 g, 91%) as a bright yellow solid.

¹H NMR (400 MHz, CDCl₃): δ ppm 6.83 (br s, 2H), 4.85 (br, 2H), 4.12 (br, 2H), 2.44(br, 2H), 1.95 (br, 2H), 1.48 (br s, 6H), 1.28 (br s, 6H); FT-IR (ATR): v=2956, 2240, 1436, 1324, 1267, 1047, 1025, 866; Anal. Calcd (%) for repeating unit [C₃₁H₂₂N₂O₅]: C 74.09, H 4.41, N 5.57, found C 73.39, H 4.44, N 5.53.

### Example Three

Membranes Casting and Pure Gas Permeation Tests

A mechanically robust isotropic film of polymer UOAPIM was cast from its chloroform solution (FIG. 32). Samples for pure-gas permeation measurements were soaked in methanol and dried before testing to remove residual solvent molecules and reverse the physical ageing effect allowing direct comparison of gas transport properties with other polymers reported in the public domain. The permeability (P) and ideal selectivity (α) of polymer UOAPIM is summarized in Table 3 below. Also given in Table 3 are the gas separation performance of currently leading PIM materials PIM-1 and SBF-PIM.

TABLE 3
Pure gas permeability and selectivity of polymer UOAPIM (31 μm) and PIM-1
control (65 μm) measured at 22° C. and a feed pressure of 50 psig. For
comparison purposes, other important PIM polymers are also listed.

| | Permeability [Barrier]^{a)} | | | | | | Ideal selectivity Pₓ/P_{y} | | | |
|---|---|---|---|---|---|---|---|---|---|---|
| Polymer | N₂ | O₂ | CO₂ | CH₄ | He | H₂ | O₂/N₂ | CO₂/CH₄ | CO₂/N₂ | H₂/N₂ |
| UOAPIM^{b)} | 980 | 3410 | 18900 | 1310 | 3880 | 9870 | 3.5 | 14.4 | 19.3 | 10.1 |
| UOAPIM^{c)} | 580 | 2210 | 14040 | 720 | 3740 | 8660 | 3.8 | 19.5 | 24.2 | 14.9 |
| PIM-1^{b)} | 540 | 1620 | 8570 | 1000 | 1660 | 4270 | 3 | 8.6 | 15.9 | 7.9 |
| PIM-1^{c)} | 415 | 1520 | 7560 | 710 | 1570 | 3880 | 3.7 | 10.6 | 18.2 | 9.3 |
| SBF-PIM^{d)} | 786 | 2640 | 13900 | 1100 | 2200 | 6320 | 3.35 | 12.6 | 17.7 | 8.1 |

^{a)}1 Barrier = 10⁻¹⁰ [cm³ (STP) cm]/(cm² s cmHg);
^{b)}fresh methanol-treated samples;
^{c)}samples aged for 24 h;
d)reported by McKeown;⁸

Of particular interest is the enhanced performance of polymer UOAPIM compared with the unlocked original PIM-1 and “improved yet not-locked” SBF-PIM. As mentioned earlier, Polymer PIM-1 is the best known PIM polymer, displaying great permeability combined with moderate selectivity. SBF-PIM is a modified version of a spiro-based PIM polymer, consisting of a bulky spirobifluorene unit that restricts the motion of polymer chains (i.e. steric hindrance is introduced). As shown in Table 3, for all tested gases, polymer UOAPIM exhibited higher permeability values than that of PIM-1 and PIM-SBF. This observation indicates that polymer UOAPIM has a larger amount of free volume and a looser space packing of the polymer chains resulting from the stiffer and more shape-persistant polymer backbone. More strikingly, as listed in Table 3, polymer UOAPIM maintained or even elevated permselectivity values compared to the corresponding values of polymer PIM-1 and PIM-SBF. The observed simultaneous improvement of both permeability and selectivity demonstrates that locking the bicyclic spiro-carbon of the SBI ring system improves the performance characteristics compared to the unlocked polymer PIM-1 and is also more effective than the alternative approach of introducing steric hindrance to improve performance.

To further evaluate polymer UOAPIM, the gas separation performance of UOAPIM was compared against data collated from different literature sources on a variety of known PIM polymers. This collated data (which includes data on PIM polymers developed by Budd and McKeown) is shown in FIGS. 33 to 36, which are the Robeson upper bound plots for gas pairs CO₂/CH₄, CO₂/N₂, H₂/N₂ and O₂/N₂, respectively, the upper bound being represented by the trade-off curves between selectivity and permeability. These trade off curves represent what was considered, at the dates indicated, as being the upper bounds for improvements in both selectivity and permeability, it being recognised that an improvement in selectivity does not necessarily mean an improvement in permeability, and vice versa. Data point 1 represents UOAPIM.

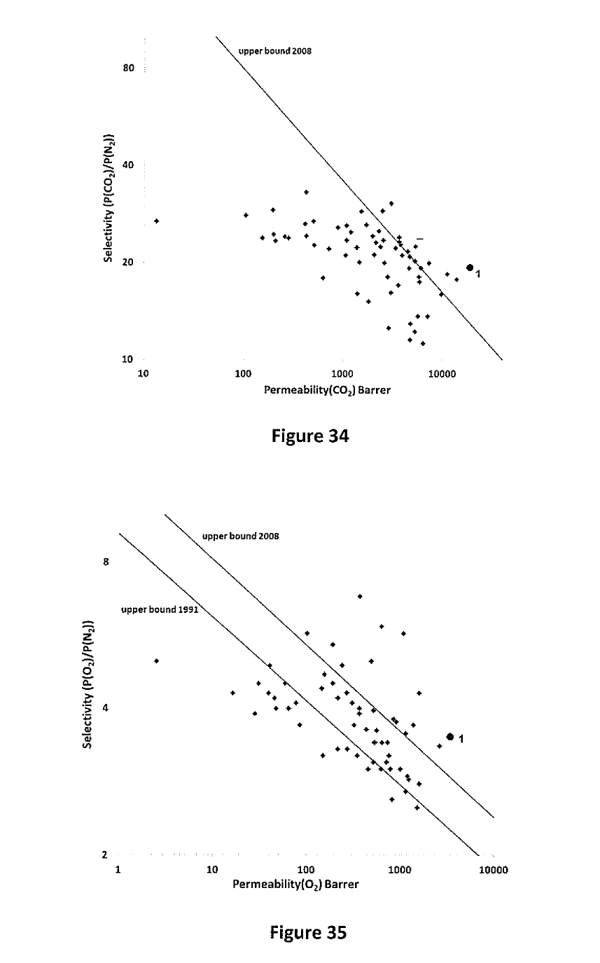

In general, polymer UOAPIM exhibits outstanding gas permeabilities within the bounds of all PIM polymers. For all tested gases, polymer UOAPIM exhibits permeability values higher than that of most polymers. For example, polymer UOAPIM has a initial permeability of 18900 Barrer for CO₂ coupled with ideal selectivities of 14.4 and 19.3 for gas pairs CO₂/CH₄ and CO₂/N₂, respectively (Table 3). FIGS. 33 and 34, which correspond to gas pairs CO₂/CH₄ and CO₂/N₂, show the longest distances achieved from the current upper bound. This advantageous performance allows potential applications in particular to commercial CO₂ removal processes, such as the industrial processes of natural gas upgrading, biogas upgrading and post-combustion CO₂ capture.

Thus, the PIM polymers including biscatechol monomer having a locked bicyclic carbon according to the present invention are very suitable for use as a material for developing gas adsorption, purification and separation membranes.

Use of Fluoride-Mediated Polymerisation in the Formation of Unlocked PIM Polymers

### Example Four

### Synthesis of 5,5′,6,6′-Tetrahydroxy-3,3,3′,3′,7,7′-hexamethylspirobisindane (1)⁸

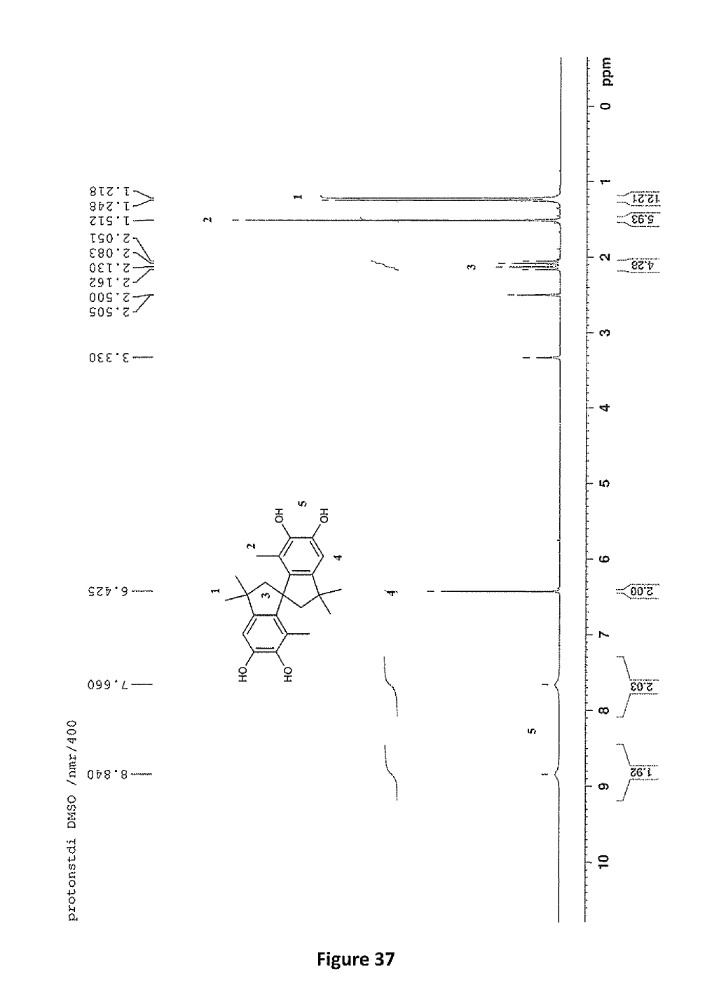

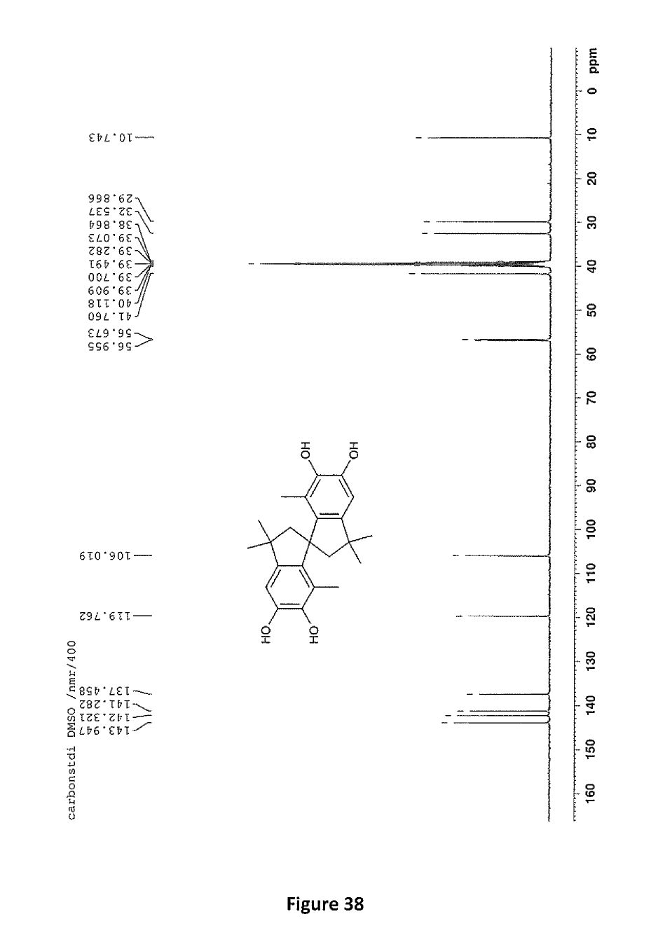

To a mixture of conc. HBr aq. (36 ml) and acetic acid (33 ml) was added 1,2-dihydroxy-3-methylbenzene (16.8 g, 135 mmol) to give a clear solution. Acetone (21 ml, 286 mmol, 2.1 eq) was then added drop wisely. After addition, the resulting solution was heated to reflux and kept refluxing for 24 hours. The hot mixture was poured into water (360 ml) with vigorous stirring. The precipitate was filtered out. Then the solid was stirred in acetic acid (150 ml) for 1 hour, filtered, and washed with acetic acid to give title compound 1 (15 g, 40.7 mmol, 60%) as a white solid. ¹H NMR (400 MHz, DMSO-d₆): δ ppm 8.83 (br, 2H), 7.67 (br, 2H), 6.43 (s, 2H), 2.16-2.05 (m, 4H), 1.51 (s, 6H), 1.25 (s, 6H), 1.22 (s, 6H) (FIG. 37); ¹³C NMR (100 MHz, DMSO-d₆): δ ppm 143.95, 142.32, 141.28, 137.46, 119.76, 106.02, 56.96, 56.67, 41.76, 32.54, 29.87, 10.74 (FIG. 38); HR-MS (ESI) calcd. 391.1880 for C₂₃H₂₈NaO₄, found 391.1868 [M+Na]⁺.

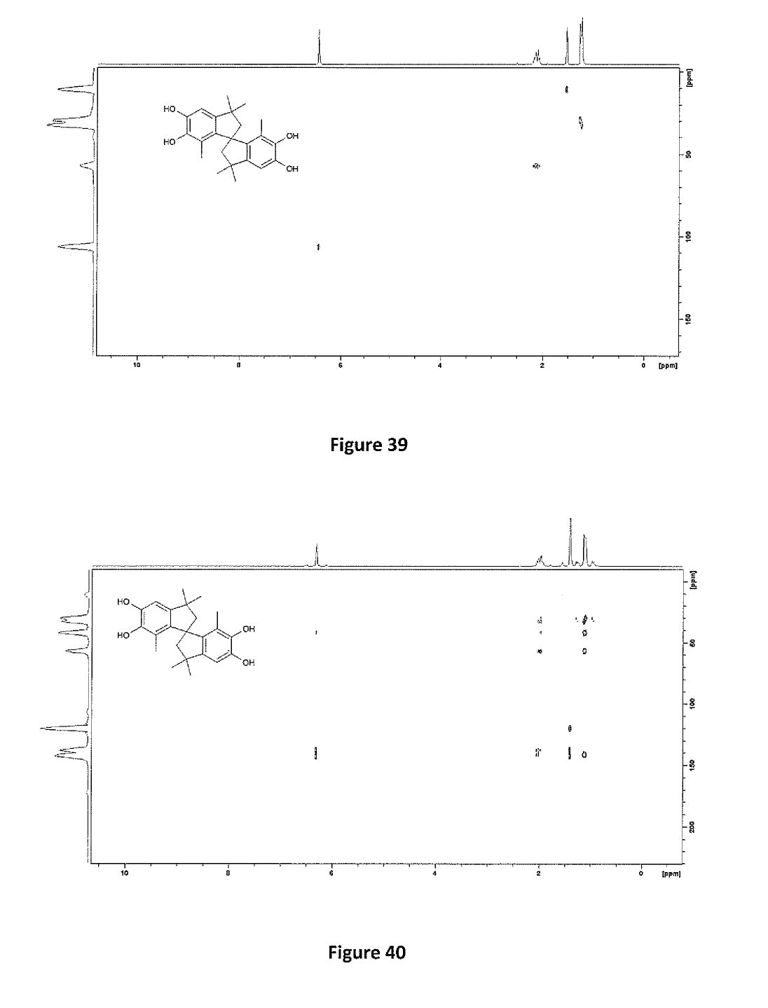

The position of the methyl group in the benzene ring of biscatechol 1 was further elucidated by ¹H-¹³C HSQC and HMBC 2D NMR (FIGS. 39 and 40).

### Example Five

### Synthesis of 5,5′,6,6′-Tetrakis(tert-butyldimethylsilyloxy)-3,3,3′,3′,7,7′-hexamethylspirobisindane(2)

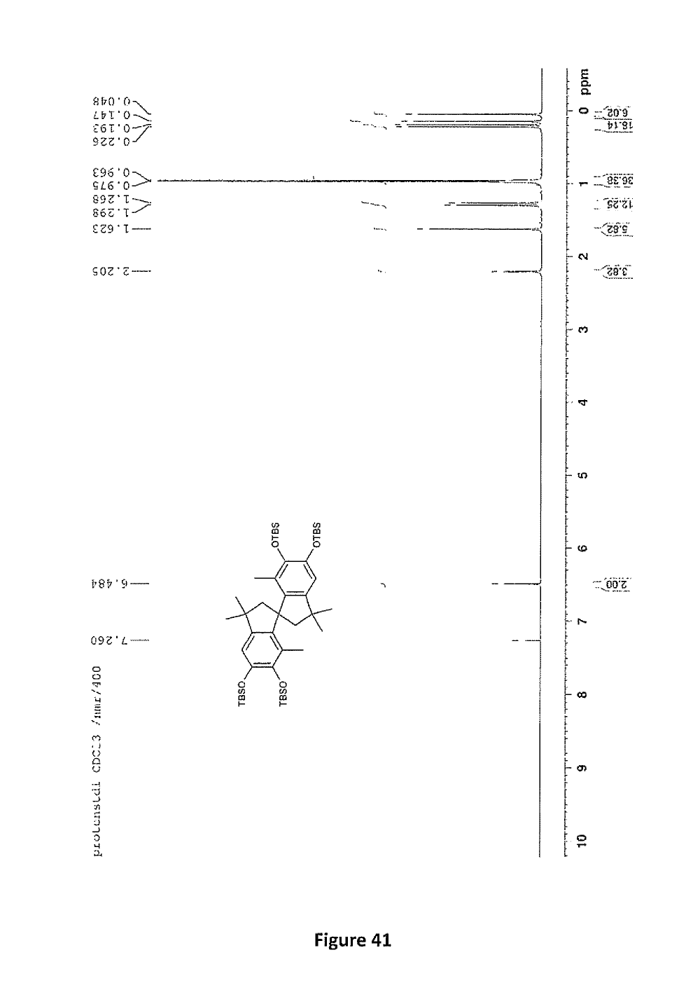

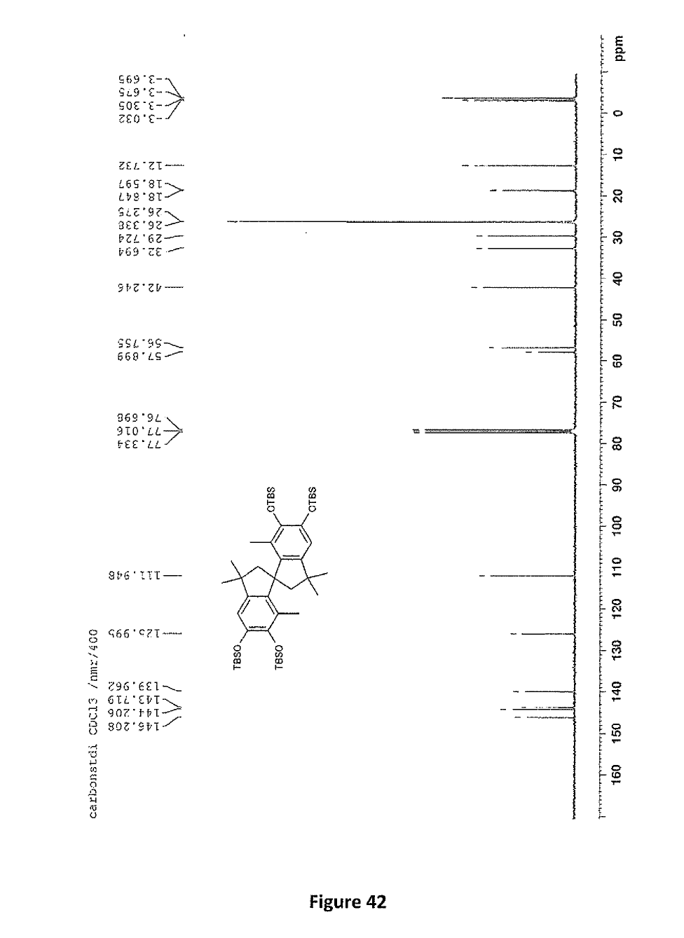

To a suspension of the bis-catechol 1 (10.85 g, 29.4 mmol) in anhydrous DMF was added t-butyl dimethyl silyl chloride (36 g, 239 mmol). The mixture was cooled down in ice-bath and added imidazole (24 g, 353 mmol) slowly within 5 minutes followed by addition of DMAP (0.36 g, 2.95 mmol). The mixture was stirred under Ar at room temperature for 3 days. Afterwards, the mixture was poured into methanol (400 ml) slowly and stirred for 1 hour. Then, the precipitate was filtered, washed with methanol and dried under vacuum to give the title compound 2 (22.6 g, 27.4 mmol, 93%) as white solid. The crude product was recrystallized from a mixture of THF and MeOH before polymerization. ¹H NMR (400 MHz, CDCl₃): δ ppm 6.48 (s, 2H), 2.24-2.17 (m, 4H), 1.62 (s, 6H), 1.29 (s, 6H), 1.27 (s, 6H), 0.97 (s, 18H), 0.96 (s, 18H), 0.22 (s, 6H), 0.19 (s, 6H), 0.14 (s, 6H), 0.05 (s, 6H) (FIG. 41); ¹³C NMR (100 MHz, CDCl₃): δ ppm 146.21, 144.21, 143.72, 139.96, 125.99, 111.95, 57.90, 56.76, 42.24, 32.69, 29.72, 26.33, 26.27, 18.84, 18.59, 12.73, −3.04, −3.31, −3.68, −3.70 (FIG. 42); HR-MS (ESI) calcd. 825.5519 for C₄₇H₈₅O₄Si₄, found 825.5540 [M+H]⁺.

### Example Six

General Procedure for Synthesis of PIM Polymer 4

A mixture of TBS-protected monomer 2 (leq.) and Tetrafluoroterephthalonitrile 3 (1 eq.) was suspended in anhydrous DMF or DMAc (solid content ca. 0.125 g total monomer weight in lml solvent). Anhydrous fluoride salt (KF or CsF) was dried in high vacuum at about 120° for 2 h before use. (TBAF solution in THF was used directly) After addition of fluoride salt at r.t., the resulting mixture was heated under argon for 72 h. (Heating procedure I: 70° C. 72 h , II: 70° C. for 6 h, then 120° C. for 66 h). Then, the reaction was cooled down, and poured into water with stirring for 1 h. The mixture was filtered, washed with water thoroughly and dried to give yellow solid as crude product 4 as a yellow solid (crude yield from 74% to quantitative.), which was used directly for GPC measurements. Prior to NMR analysis, polymers were purified via precipitation of polymer solution in CHCl₃ into MeOH. ¹H NMR (400 MHz, CDCl₃): δ ppm 6.71 (br s, 2H), 2.25 (br, 4H), 1.74 (br s, 6H), 1.35-1.32 (br m, 12H).

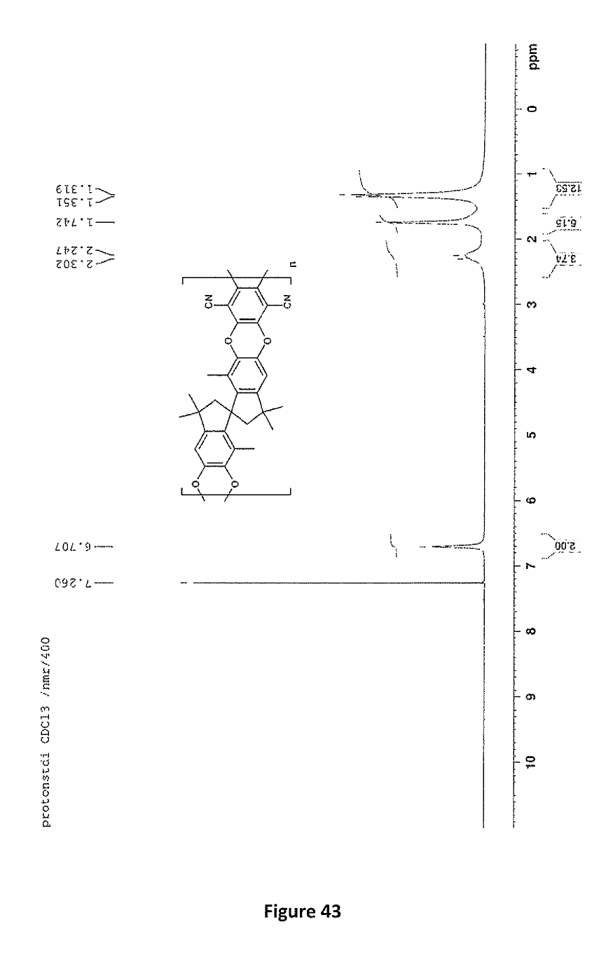

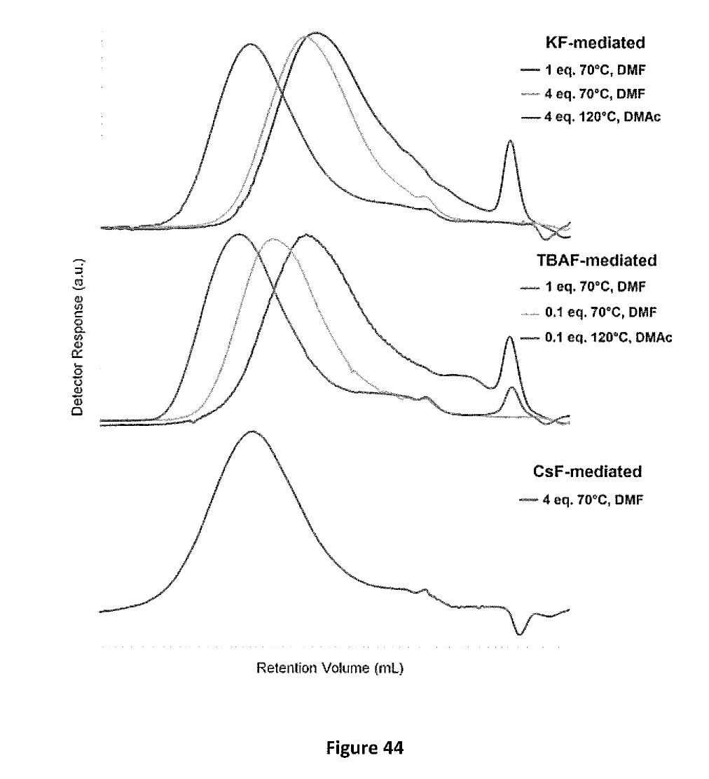

PIM Polymer 4 was found to be readily soluble in several solvents (e.g. THF, Chloroform) allowing solution NMR (FIG. 43) and Gel Permeation Chromatography (GPC) analysis (FIG. 44) in order to determine the chemical structure and the molecular weight.

### Example Seven

Demonstration of the Effectiveness of Fluoride Mediated Polymerization for the Formation of High Molecular Weight PIM Polymer Synthesis

PIM polymer 4 was prepared by the method of Example 6 under various reaction conditions and using three different fluoride ion sources, as summarized in Table 4 below.

TABLE 4
Fluoride-mediated polymerization results under different synthesis conditions

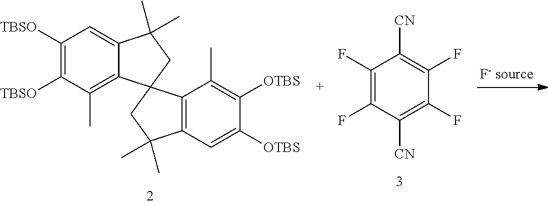 [SMILES: Cc1c(O[Si](C)(C)C(C)(C)C)c(O[Si](C)(C)C(C)(C)C)cc2c1C1(CC2(C)C)CC(C)(C)c2cc(O[Si](C)(C)C(C)(C)C)c(O[Si](C)(C)C(C)(C)C)c(C)c21 ; N#Cc1c(F)c(F)c(C#N)c(F)c1F]

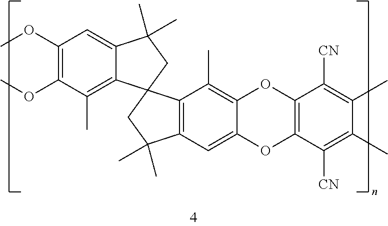

| | | Molar | | | Yield | | | M_{w}/ | Film |
|---|---|---|---|---|---|---|---|---|---|
| Entry | Catalyst | Ratio | Conditions^{b} | Solvents | % | Mₙ | M_{w} | Mₙ | Formation |
| 1 | KF | 1 eq. | 70° C. | DMF | 88 | 7,300 | 22,900 | 3.1 | No |
| 2 | KF | 4 eq. | 70° C. | DMF | 97 | 17,000 | 32,800 | 1.9 | No |
| 3 | KF | 4 eq. | 120° C. | DMAc | 99 | 44,300 | 99,700 | 2.3 | Yes |
| 4 | TBAF | 1 eq. | 70° C. | DMF | 74 | 8,100 | 30,400 | 3.8 | No |
| 5 | TBAF | 0.1 eq. | 70° C. | DMF | 93 | 29,100 | 59,700 | 2.1 | Yes |
| 6 | TBAF | 0.1 eq. | 120° C. | DMAc | 99 | 83,000 | 142,200 | 1.7 | Yes |
| 7 | CsF | 4 eq. | 70° C. | DMF | 99 | 51,600 | 113,700 | 2.2 | Yes |

Crude polymer products precipitated in water were used to conduct GPC measurements in order to determine the existence of possible cyclic oligomers (see experimental). Conditions: 70° C. for 72 h in DMF and 70° C. for 6 h, then 120° C. for 66 h in DMAc.

Yields were calculated based on the weight of crude products without removal of cyclics. Crude polymers without any re-precipitation/purification were tested for film formation tests.

As noted in Table 4, different fluoride sources were investigated and included potassium fluoride (KF), tetrabutylammonium fluoride (TBAF) and cesium fluoride (CsF). ¹H NMR spectra showed that all polymers (polymer 4) prepared via fluoride-mediated polymerization were identical. Although all three fluoride sources investigated were effective, their catalytic activities were found to depend on the solubility and hygroscopic nature of the fluoride species. Table 4 summarizes these findings based on GPC analysis (FIG. 44) and film forming test results.

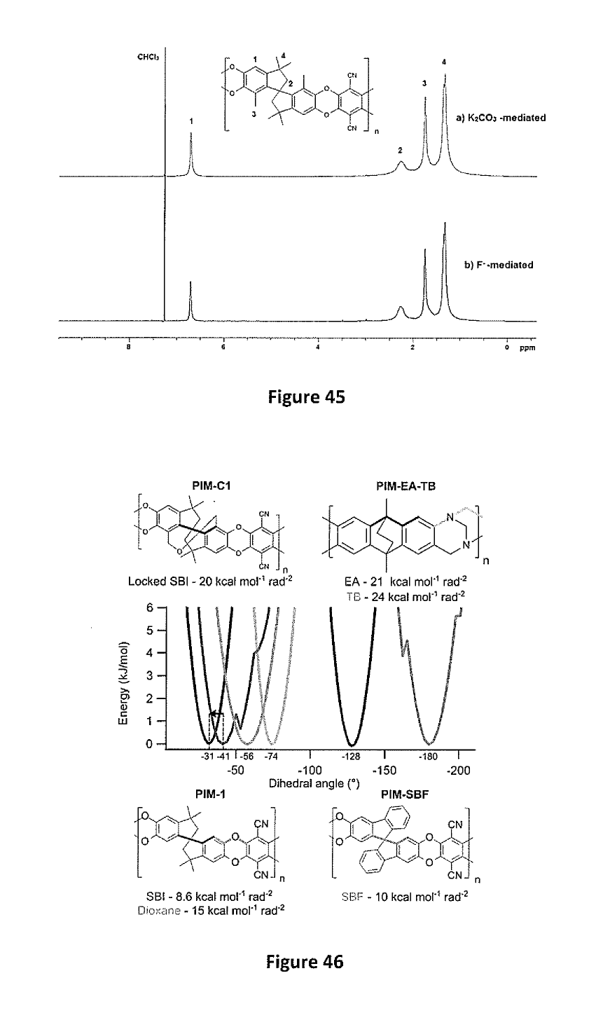

For comparison purposes, a separate batch of PIM polymer 4 was prepared via the original protocol developed by Budd and McKeown and using an un-protected biscatechol monomer 1 in the presence of K₂CO₃.¹ As shown in FIG. 45, ¹H NMR spectra of PIM polymer 4 from both methods are nearly identical, which indicates both polymers have the same chemical structures in their repeating units.

In the case of KF, a relatively inexpensive readily available fluoride ion source, two resultant PIM polymers were found to have a low molecular weight (Mₙ=7,300, M_{w}=22,900, PDI=3.1) (Entry 1 and Entry 2). As noted above, high molecular weights between 30,000 to 150,000 dalton are required for commercially useful film formation. Low molecular weight PIM polymers are too brittle for film formation. However, the inventors found that by changing the reaction solvent to DMAc and raising the temperature to 120° C., a high-molecular weight polymer was obtained which could be cast into a flexible free-standing film (Mₙ=44,300, M_{w}=99,700, PDI=2.3) (Entry 3).

Tetrabutylammonium fluoride (TBAF), a soluble organic fluoride salt, has good solubility in aprotic polar solvents (e.g. DMF, DMAc). The inventors therefore hypothesized that TBAF could have an advantage on catalytic efficiency over inorganic KF. However, the product of Entry 4 again had a low molecular weight with high PDI value (Mₙ=8,100, M_{w}=30,400, PDI=3.8) similar to Entry 1. Since the commercial TBAF solution in THF has an approximately 5 wt. % water content, the side reaction produced dead oligomers, hence considerably impaired molecular weight with broadened PDI value. In order to suppress the hydrolysis of aryl fluoride, a smaller amount of TBAF was used in Entry 5, and a much higher molecular weight polymer was obtained which was able to fabricate self-standing films (Mₙ=29,100, M_{w}=59,700, PD2.1). The polymerization was further modified by stirring at 120° C. in DMAc and a polymer with even higher molecular weight was obtained (Mₙ=83,000, M_{w}=142,200, PDI=1.7) (Entry 6). Thus, Entry 6 seems to suggest the best reaction conditions.

CsF is partially soluble in DMF and readily accessible as an anhydrous fluoride source, which encouraged the inventors to use it in the new polymerization method. Polymerization was conducted by use of 4 equivalent CsF afford a high molecular weight product (Mₙ=51,600, M_{w}=113,700, PDI=2.2), which can form strong self-standing film as expected (Entry 7).

Thus, by optimizing reaction conditions, the inventors were able to obtain PIM polymers capable of forming free-standing films with each of KF, TBAF and CsF. Each fluoride source investigated gave high molecular weight PIM polymer 4 with desirable polydispersity. Fluoride-mediated polymerization therefore provides a commercially useful mild synthetic route to the formation of PIM polymers.

It is noteworthy that all samples subjected to GPC analysis were obtained from the direct precipitation of reaction mixture in water. Here, the motivation was to capture the entire composition of the product prepared by this fluoride-mediated method. As shown in FIG. 11, high molecular weight film forming polymers 4 were obtained with moderate polydispersity (Entry 3, 5, 6 and 7) even without the re-dissolving/precipitation step. This evidence strongly suggests that this polymerization method has a low tendency of cyclic oligomer formation. Cyclic oligomers are usually removed during the polymer formation process as they impair the resulting polymer's mechanical properties.

As is apparent from the above results, all three fluoride ion sources investigated (KF, TBAF and CsF) can effectively catalyze the polymerization reaction between a TBS-protected biscatechol monomer 2 (see FIG. 15) and an activated tetrafluoro monomer 3 (see synthetic route in Table 4). As will be apparent to a skilled person, the invention is not intended to be limited to these particular examples. This new method can serve as an alternative PIMs polymerization protocol to known options.

### Example Eight

Increased Rigidity of PIM Polymers Having a Locked SBI Unit

To quantify the rigidity of a locked SBI unit (indicated as PIM-C1 in FIG. 46 and which is equivalent to UOAPIM referred to earlier in respect of Examples 2 and 3) according to the present invention and to compare it with other high-performance PIMs (indicated as PIM-1, PIM-EA-TB and PIM-SBF in FIG. 46), a full quantum mechanical approach using density functional theory was used.

FIG. 46 shows a plot of dihedral potential energy surfaces for different high-performance PIM moieties. It can be seen that the equilibrium dihedral angle of the SBI structure is shifted to the left after locking (from −41° for unlocked SBI to −31° for locked SBI). Such a reduced dihedral angle may alter the polymer free volume size and micropore geometry.

To quantify the difference in the structural rigidities of the different moieties, the curvature of the dihedral potential energy surface was calculated by fitting a harmonic model to the surface. The spring constant is taken as a rigidity parameter with units of kcal mol⁻¹ rad⁻². Energetically, locking the SBI unit substantially increases the rigidity parameter value of the spiro-carbon by 230% relative to the unlocked version (20 kcal mol⁻¹ rad⁻² for the locked SBI unit (PIM-C1) compared with 8.6 kcal mol⁻¹ rad⁻² for the unlocked SBI unit (PIM-1)). The rigidity of the locked SBI unit (PIM-C1) is also found to be close to that of the Troger's base (TB) and ethano dihydroanthracene (EA) structures (21 kcal mol⁻¹ rad⁻² for EA and 24 kcal mol⁻¹ rad⁻² for TB), but is slightly more than PIM-SBF (10 kcal mol ⁻¹ rad⁻²).

Molecular dynamics simulations were also employed to thermally excite two simplified oligomeric chains containing only six units of either SBI (PIM-1) or locked SBI (PIM-C1).

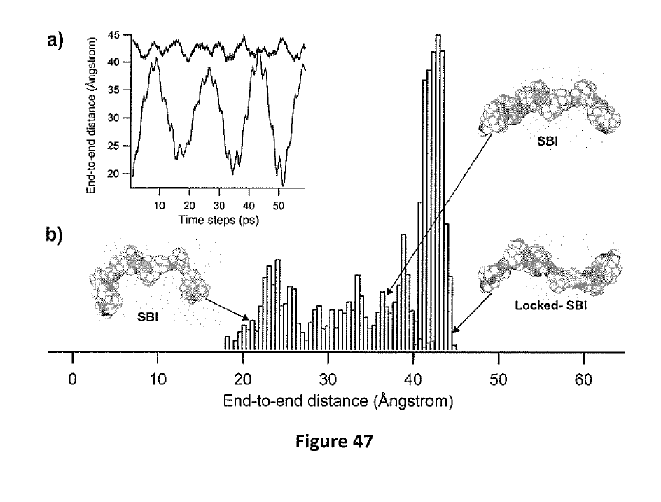

FIG. 47a plots the time resolved end-to-end distance change and shows the SBI oligomer oscillating between the two conformations with a time period of about 20 ps, while the locked SBI oscillations are higher in frequency with a lower period of about 7 ps. This behaviour is indicative of greater rigidity for the locked SBI than the unlocked SBI.

FIG. 47b is a histogram showing the distribution of accessible end-to end distances for the oligomers with the locked and unlocked SBI units. The chain having six locked SBI units not only leads to a more stretched conformation compared with the unlocked SBI structures, but also maintains a very tight end-to-end distance within the 40 to 45 Angstrom range, while the unlocked SBI travels between 20 and 43 Angstroms indicating various accessible conformations.

The results indicate that the locked SBI unit (PIM-C1) is twice as rigid as the base SBI structure (PIM-1) and that the locking improves the rigidity of the base PIM unit such that it is equivalent to some of the most successful high performance building blocks utilized for the purpose of obtaining a rigid PIM polymer backbone, including EA, TB and PIM-SBF, as previously mentioned herein in the Background section.

The forgoing describes the invention including preferred forms thereof. Alterations or modifications that would be apparent to a person skilled in the art are intended to be included in the scope of the invention.

Reference to any prior art in this specification is not intended to acknowledge that the art is common general knowledge of a person skilled in the art in any particular country.

Reference to C₁ -C₆ herein is intended to include reference to each of C₁, C₂, C₃, C₄, C₅ and C₆.

The Following Paragraphs Describe the Present Invention:
1. A method of increasing the rigidity of PIM homo- or co-polymers including repeating units containing at least one biscatechol monomer having a fused spiro-bisindane ring system of Formula (I), the spiro-bisindane ring system (SBI) including a bicyclic spiro-carbon:

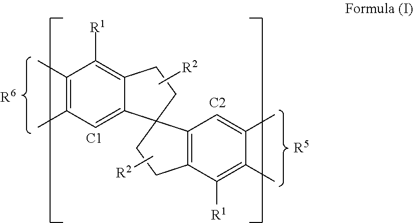

wherein
each R¹ can be the same or different, each R² can be the same or different and wherein R¹ and/or R² are selected from any one or more of H; straight or branched, saturated or unsaturated lower C₁-C₆ alkyl groups, wherein the lower alkyl groups can include aromatic or non-aromatic ring structures; R³OR⁴, R³O(C═O)R⁴, R³C(═O)OR⁴, or R³OH, wherein each of R³ and R⁴ are the same or different and are selected from straight or branched, saturated or unsaturated lower C₁-C₆ alkyl groups; and
R⁵ and R⁶ represent suitable linking monomers, wherein the linking monomers can be the same or different and are preferably selected from any one or more tetrahalo aromatic monomers;
and wherein the method includes the step of introducing an intra-molecular lock between C1 and C2 of the biscatechol monomer of Formula (I).
2. The method as described in paragraph 1, wherein the intramolecular lock between C1 and C2 forms:
   (a) an 8 membered ring structure including —CH₂—Y—CH₂—, and wherein Y is O; CH₂; C(═O); C(OR⁷)(OR⁸); NH; NR⁹; S; S(═O); SO₂; C(═S); PH; PR¹⁰; BH; BR¹¹ or BOR¹¹; and wherein R⁷ and R⁸ are selected from any one or more of H or C₁-C₆ alkyl groups; and wherein R⁹, R¹⁰and R¹¹are selected from any one or more of H or straight or branched, saturated or unsaturated lower C₁-C₆ alkyl groups, wherein the lower alkyl groups can include aromatic or non-aromatic ring structures; or
   (b) a 7 membered ring structure including —CH₂-CH₂—, —CH═CH—, —(═O)O—, —C(═O)NH- or —CHR¹²-CHR¹²—, wherein R¹²are selected from any one or more of H or straight or branched, saturated or unsaturated lower C₁-C₆ alkyl groups, wherein the lower alkyl groups can include aromatic or non-aromatic ring structures.
3. The method as described in paragraph 1 or 2 wherein each R² is dimethyl and the biscatechol monomer of Formula (I) is:

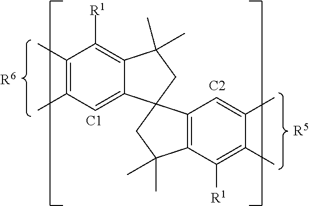

wherein the biscatechol monomer contains a fused spiro-bisindane ring system (SBI).
4. The method as described in paragraph 3, wherein each R¹ is H.
5. The method as described in paragraph 1 or 2, wherein each R² is a C₆ aromatic ring and the biscatechol monomer of Formula (I) is:

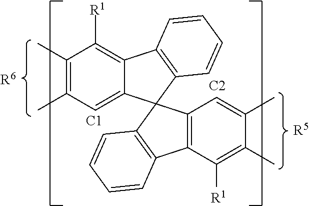

wherein the biscatechol monomer contains a fused spiro-bisfluorene ring system (SBF).
6. The method as described in any one of paragraphs 1 to 5, wherein the suitable tetrahalo aromatic monomer is a tetrafluoro, tetrabromo, tetrachloro or tetraiodo aromatic monomer.
7. The method as described in paragraph 6, wherein the tetrahalo aromatic monomer is a tetrafluoro aromatic monomer.
8. A method of preparing a biscatechol monomer of Formula (V), the biscatechol monomer including a fused spiro-bisindane ring system (SBI) having a locked bicyclic spiro-carbon, wherein the method includes the steps of:
   (a) protecting each hydroxyl group of a dimethyl substituted biscatechol monomer of Formula (II) having a bicyclic spiro-carbon with a silyl ether protecting group to give a protected dimethyl substituted biscatechol monomer of Formula (III):

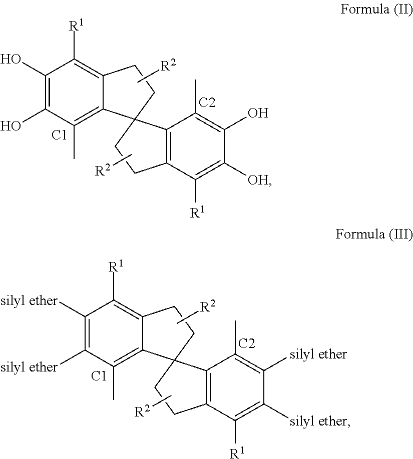

wherein the silyl ether protecting groups are the same or different; and
wherein, R¹ and R² are as defined previously for Formula (I) and
(b) halogenating the silyl ether protected biscatechol monomer of Formula (III) from step (a) to form a silyl ether protected bis(halomethyl) substituted biscatechol monomer of Formula (IV), wherein the halide ion employed in the halogenation is selected from bromide, chloride or iodide ions:

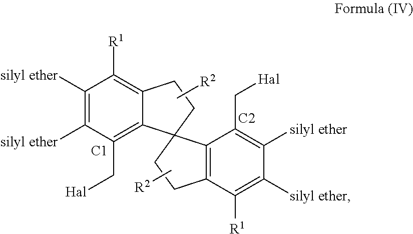

wherein R¹ and R² and silyl ether are as defined previously for Formula (III), and
wherein Hal is any one of bromide, chloride or iodide ions, and
(c) (i) dehalogenating the biscatechol monomer of Formula (IV) and forming a covalent, intramolecular lock between C1 and C2 of the protected biscatechol monomer of Formula (IV) with a suitable locking group X; or
   (ii) substituting the halide ions in Formula (IV) with hydroxyl groups, followed by cyclised dehydration to form a covalent, intramolecular lock between C1 and C2 of the protected biscatechol monomer of Formula (IV);
   to provide a silyl ether protected biscatechol monomer of Formula (V) having a locked bicyclic spiro-carbon:

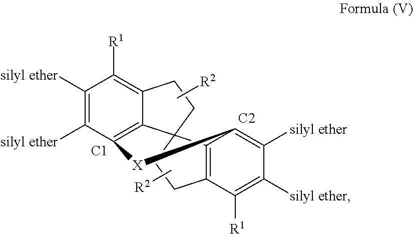

wherein, R¹ and R² and silyl ether are as defined previously for Formula (III), and
(i) X is —CH₂—Y—CH₂—, and wherein Y is O; CH₂; C(═O); C(OR⁷)(OR⁸); NH; NR⁹; S; S(═O); SO₂; C(═S); PH; PR¹⁰; BH; BR¹¹ or BOR¹¹;
   and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or
(ii) X is —CH₂-CH₂—, —CH═CH—, —(═O)O—, —C(═O)NH— or —CHR¹²-CHR¹²—, wherein R¹² are selected from any one or more of H or straight or branched, saturated or unsaturated lower C₁-C₆ alkyl groups, wherein the lower alkyl groups can include aromatic or non-aromatic ring structures.
9. In an alternative to the method of paragraph 8 for preparing a biscatechol monomer of Formula (V), the biscatechol monomer including a fused spiro-bisindane ring system (SBI) having a bicyclic spiro-carbon, wherein the alternative method includes the steps of:
   (a) halogenating a silyl ether protected biscatechol monomer of Formula (III) to form a silyl ether protected bis(halomethyl) substituted biscatechol monomer of Formula (IV), wherein the halide ion employed in the halogenation is selected from bromide, chloride or iodide ions:

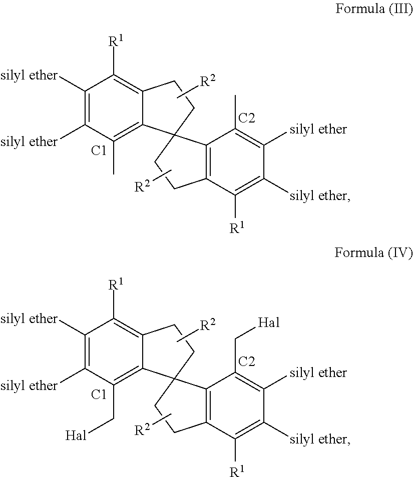

wherein, the silyl ether protecting groups are the same or different, and
wherein, R¹ and R² are as defined previously for Formula (I), and wherein Hal is any one of bromide, chloride or iodide ions, and
(b) (i) dehalogenating the biscatechol monomer of Formula (IV) and forming a covalent, intramolecular lock between C1 and C2 of the protected biscatechol monomer of Formula (IV) with a suitable locking group X; or
   (ii) substituting the halide ions in Formula (IV) with hydroxyl groups, followed by cyclised dehydration to form a covalent, intramolecular lock between C1 and C2 of the protected biscatechol monomer of Formula (IV);
   to provide a silyl ether protected biscatechol monomer of Formula (V) having a locked bicyclic spiro-carbon:

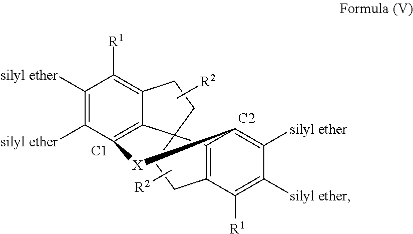

wherein, R¹ and R² and silyl ether are as defined previously for Formula (V), and
(i) X is —CH₂—Y—CH₂—, and wherein Y is O; CH₂; C(═O); C(OR⁷)(OR⁸); NH; NR⁹; S; S(═O); SO₂; C(═S); PH; PR¹⁰; BH; BR¹¹ or BOR¹¹;
   and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or
(ii) X is —CH₂-CH₂—, —CH═CH—, —(═O)O—, —C(═O)NH— or —CHR¹²-CHR¹²—, wherein R¹² are selected from any one or more of H or straight or branched, saturated or unsaturated lower C₁-C₆ alkyl groups, wherein the lower alkyl groups can include aromatic or non-aromatic ring structures.
10. The method as described in paragraph 8 or 9, wherein the silyl ether protecting group is selected from Formula (VI):

R¹³R¹⁴R¹⁵Si—O−R¹⁶ (VI)
wherein R¹³ to R¹⁶ are alkyl groups or aryl groups.
11. The method as described in paragraph 10, wherein the silyl ether protecting group is selected from any one or more of trimethylsilyl (TMS), tert-butyldiphenylsilyl (TBDPS), tert-butyldimethylsilyl (TBS) and triisopropylsilyl (TIPS).
12. The method as described in any one of paragraphs 8 to 11, wherein the dehalogenation step to form an intramolecular lock between C1 and C2 is catalysed by a transition metal salt, metal oxide and/or a pure metal.
13. The method as described in paragraph 12, wherein the transition metal salt is a silver(I), iron(III), titanium(II) or tin(II) salt.
14. The method as described in paragraph 13, wherein the silver(I) salt is AgNO₃ or Ag₂CO₃.
15. The method as described in paragraph 13, wherein the metal oxide is ZnO or Ag₂O.
16. The method as described in paragraph 13, wherein the pure metal is zinc.
17. The method as described in any one of paragraphs 8 to 16, wherein each R² is a dimethyl and the biscatechol monomer of Formula (V) contains a fused SBI ring system.
18. The method as described in any one of paragraphs 8 to 16, wherein each R² is a C₆ aromatic ring and the biscatechol monomer of Formula (V) contains a fused SBF ring system.
19. A silyl ether protected biscatechol monomer of Formula (IV), the biscatechol monomer including a fused spiro-bisindane ring system (SBI) having a bicyclic spiro-carbon:

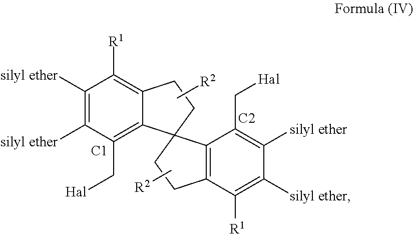

wherein
each R¹ can be the same or different, each R² can be the same or different and wherein R¹ and/or R² are selected from any one or more of H; straight or branched, saturated or unsaturated lower C₁-C₆ alkyl groups, wherein the lower alkyl groups can include aromatic or non-aromatic ring structures R³OR⁴, R³O(C═O)R⁴, R³C(═O)OR⁴, or R³OH,
wherein each of R³ and R⁴ are the same or different and are selected from straight or branched, saturated or unsaturated lower C₁-C₆ alkyl groups; and
wherein the silyl ether groups are the same or different.
20. The silyl ether protected biscatechol monomer as described in paragraph 19, wherein Hal represents any one of bromide, chloride or iodide ions.
21. A silyl ether protected biscatechol monomer of Formula (V), the biscatechol monomer including a fused spiro-bisindane ring system (SBI) having a bicyclic spiro-carbon:

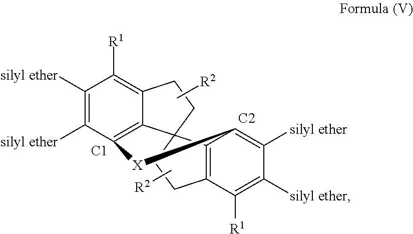

wherein,
each R¹ can be the same or different, each R² can be the same or different and wherein R¹ and/or R² are selected from any one or more of H; straight or branched, saturated or unsaturated lower C₁-C₆ alkyl groups, wherein the lower alkyl groups can include aromatic or non-aromatic ring structures R³OR⁴, R³O(C═O)R⁴, R³C(═O)OR⁴, or R³OH,
wherein each of R³ and R⁴ are the same or different and are selected from straight or branched, saturated or unsaturated lower C₁-C₆ alkyl groups; and
wherein,
   (i) X is —CH₂—Y—CH₂—, and wherein Y is O; CH₂; C(═O); C(OR⁷)(OR⁸); NH; NR⁹; S; S(═O); SO₂; C(═S); PH; PR¹⁰; BH; BR¹¹ or BOR¹¹;
      and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or
   (ii) X is —CH₂-CH₂—, —CH═CH—, —(═O)O—, —C(═O)NH— or —CHR¹²-CHR¹²—, wherein R¹² are selected from any one or more of H or straight or branched, saturated or unsaturated lower C₁-C₆ alkyl groups, wherein the lower alkyl groups can include aromatic or non-aromatic ring structures ; and
wherein the silyl ether protecting groups are the same or different.
22. The silyl ether protected biscatechol monomer as described in any one of paragraphs 19 to 21, wherein the silyl ether protecting group is selected from Formula (VI):

R¹³R¹⁴R¹⁵Si—O—R¹⁶ (VI)
wherein R¹³ to R¹⁶ are alkyl groups or aryl groups.
23. The silyl ether protected biscatechol monomer as described in paragraph 22, wherein the silyl ether protecting group is selected from any one or more of trimethylsilyl (TMS), tert-butyldiphenylsilyl (TBDPS), tert-butyldimethylsilyl (TBS) and triisopropylsilyl (TIPS).
24. The silyl ether protected biscatechol monomer as described in any one of paragraphs 19 to 23, wherein each R² is a dimethyl and the biscatechol monomer contains a fused SBI ring system.
25. The silyl ether protected biscatechol monomer as described in any one of paragraphs 19 to 23, wherein each R² is a C₆ aromatic ring and the biscatechol monomer contains a fused SBF ring system.
26. A method of preparing a biscatechol monomer of Formula (VII), the biscatechol monomer including a fused spiro-bisindane ring system (SBI) having a locked bicyclic spiro-carbon, wherein the method includes the steps of deprotecting a silyl ether protected biscatechol monomer of Formula (V) using a fluoride ion source to form a biscatechol monomer of Formula (VII) having a locked bicyclic carbon:

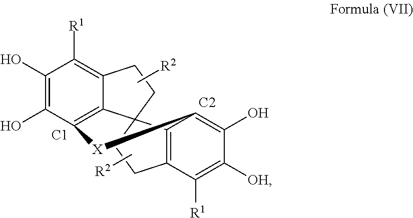

wherein,
   (i) X is —CH₂—Y—CH₂—, and wherein Y is O; CH₂; C(═O); C(OR⁷)(OR⁸); NH; NR⁹; S; S(═O); SO₂; C(═S); PH; PR¹⁰; BH; B R¹¹ or BO R¹¹;
      and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or
   (iii) X is —CH₂-CH₂—, —CH═CH—, —(═O)O—, —C(═O)NH— or —CHR¹²-CHR¹²—, wherein R¹² are selected from any one or more of H or straight or branched, saturated or unsaturated lower C₁-C₆ alkyl groups, wherein the lower alkyl groups can include aromatic or non-aromatic ring structures; and
   (iv) R¹ and R² are as defined for Formula (V).
27. The method as described in paragraph 26, wherein the fluoride ion source is tetrabutylammonium fluoride (TBAF).
28. The method as described in paragraph 26 or 27, wherein each R² is a dimethyl and the biscatechol monomer contains a fused SBI ring system.
29. The method as described in paragraph 26 or 27, wherein each R² is a C₆ aromatic ring and the biscatechol monomer contains a fused SBF ring system.
30. A biscatechol monomer of Formula (VII), the biscatechol monomer including a fused spiro-bisindane ring system (SBI) having a locked bicyclic spiro-carbon:

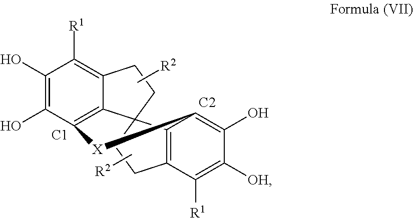

wherein,
each R¹ can be the same or different, each R² can be the same or different and wherein R¹ and/or R² are selected from any one or more of H; straight or branched, saturated or unsaturated lower C₁-C₆ alkyl groups, wherein the lower alkyl groups can include aromatic or non-aromatic ring structures R³OR⁴, R³O(C═O)R⁴, R³C(═O)OR⁴, or R³OH, wherein each of R³ and R⁴ are the same or different and are selected from straight or branched, saturated or unsaturated lower C₁-C₆ alkyl groups; and
   (i) X is —CH₂—Y—CH₂—, and wherein Y is O; CH₂; C(═O); C(OR⁷)(OR⁸); NH; NR⁹; S; S(═O); SO₂; C(═S); PH; PR¹⁰; BH; BR¹¹ or BOR¹¹;
      and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or
   (ii) X is —CH₂-CH₂—, —CH═CH—, —(═O)O—, —C(═O)NH— or —CHR¹²-CHR¹²—, wherein R¹² are selected from any one or more of H or straight or branched, saturated or unsaturated lower C₁-C₆ alkyl groups, wherein the lower alkyl groups can include aromatic or non-aromatic ring structures.
31. The biscatechol monomer as described in paragraph 30, wherein each R² is a dimethyl and the biscatechol monomer contains a fused SBI ring system.
32. The biscatechol monomer as described in paragraph 30, wherein each R² is a C₆ aromatic ring and the biscatechol monomer contains a fused SBF ring system.
33. A use of a biscatechol monomer of Formula (V) or Formula (VII) in the preparation of a PIM homo- or co-polymer.
34. A fluoride-mediated double nucleophilic aromatic substitution polycondensation (or polymerisation) method for the preparation of a PIM polymer, wherein the fluoride-mediated polymerisation is between a biscatechol monomer and a tetrafluoro aromatic monomer, and wherein the hydroxyl groups on the biscatechol monomer are protected by one or more silyl ether protecting groups.
35. The fluoride-mediated method as described in paragraph 34, wherein the silyl ether protecting group is selected from Formula (VI):

R¹³R¹⁴R¹⁵Si—O—R¹⁶ (VI)
wherein R¹³ to R¹⁶ are alkyl groups or aryl groups.
36. The fluoride-mediated method as described in paragraph 35, wherein the silyl ether protecting group is selected from any one or more of trimethylsilyl (TMS), tert-butyldiphenylsilyl (TBDPS), tert-butyldimethylsilyl (TBS) and triisopropylsilyl (TIPS).
37. The fluoride-mediated method as described in any one of paragraphs 34 to 36, wherein the tetrafluoro aromatic monomer is 2,3,5,6-tetrafluoroterephthalonitrile.
38. The fluoride-mediated method as described in any one of paragraphs 34 to 37, wherein fluoride mediation is provided by organic or inorganic fluoride ion sources, wherein the fluoride ions are sourced from any one or more of potassium fluoride (KF), tetrabutylammonium fluoride (TBAF), cesium fluoride (CsF), organic quaternary ammonium fluoride salts, phosphonium fluoride and other inorganic fluoride salts.
39. The fluoride-mediated method as described in any one of paragraphs 34 to 38, wherein sufficient fluoride ions are provided to meet at least a 0.001 molar ratio equivalence of fluoride ions to silyl ether protecting groups present to catalyse the reaction between the biscatechol monomer and the tetrafluoro aromatic monomer.
40. The fluoride-mediated method as described in any one of paragraphs 34 to 39, wherein the biscatechol monomer is a biscatechol monomer of Formulae (III), (IV) or (V).
41. The use of fluoride ions in a fluoride-mediated double nucleophilic aromatic substitution polymerization method for the manufacture of PIM polymers from a biscatechol monomer and a tetrafluoro aromatic monomer, wherein hydroxyl groups on the biscatechol monomer are protected by one or more silyl ether protecting groups.
42. The use as described in paragraph 41, wherein the silyl ether protecting group is selected from Formula (VI):

R¹³R¹⁴R¹⁵Si—O—R¹⁶ (VI)
wherein R¹³ to R¹⁶ are alkyl groups or aryl groups.
43. The use as described in paragraph 41, wherein the silyl ether protecting groups are the same.
44. The use as described in paragraph 41 or 42, wherein the silyl ether protecting groups are selected from any one or more of trimethylsilyl (TMS), tert-butyldiphenylsilyl (TBDPS), tert-butyldimethylsilyl (TBS) and triisopropylsilyl (TIPS).
45. The use as described in any one of paragraphs 41 to 44, wherein the tetrafluoro aromatic monomer is 2,3,5,6-tetrafluoroterephthalonitrile.
46. The use as described in any one of paragraphs 41 to 45, wherein the fluoride ions are provided by organic or inorganic fluoride ion sources, wherein the fluoride ions are sourced from any one or more of potassium fluoride (KF), tetrabutylammonium fluoride (TBAF), cesium fluoride (CsF), organic quaternary ammonium fluoride salts, phosphonium fluoride and other inorganic fluoride salts.
47. The use as described in any one of paragraphs 41 to 46, wherein sufficient fluoride ions are provided to meet at least a 0.001 molar ratio equivalence of fluoride ions to silyl ether protecting groups present to catalyse the reaction between the biscatechol monomer and the tetrafluoro aromatic monomer.
48. The use as described in any one of paragraphs 41 to 47, wherein the biscatechol monomer is a biscatechol monomer of Formulae (III), (IV) or (V).
49. A method for the synthesis of a PIM polymer, the method including a fluoride-mediated double nucleophilic aromatic substitution polymerization of a biscatechol monomer with a tetrafluoro aromatic monomer, and wherein hydroxyl groups of the biscatechol monomer are protected by one or more silyl ether protecting groups.
50. The method as described in paragraph 49, wherein the silyl ether protecting group is selected from Formula (VI):

R¹³R¹⁴R¹⁵Si—O—R¹⁶ (VI)
wherein R¹³ to R¹⁶ are alkyl groups or aryl groups.
51. The method as described in paragraph 50, wherein the silyl ether protecting groups are the same.
52. The method as described in paragraph 50 or 51, wherein the silyl ether protecting groups are selected from any one or more of trimethylsilyl (TMS), tert-butyldiphenylsilyl (TBDPS), tert-butyldimethylsilyl (TBS) and triisopropylsilyl (TIPS).
53. The method as described in any one of paragraphs 49 to 52, wherein the tetrafluoro aromatic monomer is 2,3,5,6-tetrafluoroterephthalonitrile.
54. The method as described in any one of paragraphs 49 to 53, wherein the fluoride ions are provided by organic or inorganic fluoride ion sources, wherein the fluoride ions are sourced from any one or more of potassium fluoride (KF), tetrabutylammonium fluoride (TBAF), cesium fluoride (CsF), organic quaternary ammonium fluoride salts, phosphonium fluoride and other inorganic fluoride salts.
55. The method as described in any one of paragraphs 49 to 54, wherein sufficient fluoride ions are provided to meet at least a 0.001 molar ratio equivalence of fluoride ions to silyl ether protecting groups present to catalyse the reaction between the biscatechol monomer and the tetrafluoro aromatic monomer.
56. The method as described in any one of paragraphs 49 to 55, wherein the biscatechol monomer is a biscatechol monomer of Formulae (III), (IV) or (V).
57. A PIM polymer prepared by a method or use as described in any one of paragraphs 34 to 56.
58. A method of preparing a PIM homo-polymer, wherein the method includes the step of reacting a biscatechol monomer of Formula (V) or Formula (VII) with a suitable linking monomer.
59. The method as described in paragraph 58, wherein the suitable linking monomer is a tetrahalo aromatic monomer selected from a tetrafluoro, tetrabromo, tetrachloro or tetraiodo aromatic monomer.
60. The method as described in paragraph 58 or 59, wherein the biscatechol monomer of Formula (V) or Formula (VII) is reacted with the suitable linking monomer in the presence of an organic base, an inorganic base and/or fluoride ions.
61. The method as described in paragraph 60, wherein the biscatechol monomer is of Formula (V) and is base sensitive and is reacted with the suitable linking monomer in the presence of fluoride ions.
62. The method as described in paragraph 61, wherein the fluoride ions are provided by organic or inorganic fluoride ion sources, wherein the fluoride ions are sourced from any one or more of potassium fluoride (KF), tetrabutylammonium fluoride (TBAF), cesium fluoride (CsF), organic quaternary ammonium fluoride salts, and other inorganic fluoride salts.
63. The method as described in paragraph 60, wherein the biscatechol monomer is of Formula (V) or Formula (VII) and is not base sensitive and is reacted with the suitable linking monomer in the presence of an organic or inorganic base.
64. The method as described in paragraph 63, wherein the organic base is a non-nucleophilic organic base, preferably a non-nucleophilic organic base containing nitrogen.
65. The method as described in paragraph 64, wherein the nitrogen containing base is trimethylamine.
66. The method as described in paragraph 63, wherein the inorganic base is a carbonate salt, preferably K₂CO₃.
67. A method of preparing a PIM co-polymer, wherein the method includes the step of reacting together (i) a first biscatechol monomer of Formula (V) or Formula (VII), (ii) a second biscatechol monomer that is different to the first biscatechol monomer, and (iii) a suitable linking monomer.
68. The method as described in paragraph 67, wherein the second biscatechol monomer is a biscatechol monomer of Formula (V) or Formula (VII).
69. The method as described in paragraph 67 or 68, wherein the suitable linking monomer is a tetrahalo aromatic monomer selected from a tetrafluoro, tetrabromo, tetrachloro or tetraiodo aromatic monomer.
70. The method as described in any one of paragraphs 67 to 69, wherein the first biscatechol monomer, and the second biscatechol monomer, and the suitable linking monomer are reacted together in the presence of an organic base, an inorganic base and/or fluoride ions.
71. The method as described in paragraph 70, wherein the first biscatechol monomer is of Formula (V) and the first and second biscatechol monomer are base sensitive and are reacted with the suitable linking monomer in the presence of fluoride ions.
72. The method as described in paragraph 71, wherein the fluoride ions are provided by organic or inorganic fluoride ion sources, wherein the fluoride ions are sourced from any one or more of potassium fluoride (KF), tetrabutylammonium fluoride (TBAF), cesium fluoride (CsF), organic quaternary ammonium fluoride salts, and other inorganic fluoride salts.
73. The method as described in paragraph 70, wherein the first and second biscatechol monomer is not base sensitive and is reacted with the suitable linking monomer in the presence of an organic or inorganic base.
74. The method as described in paragraph 73, wherein the organic base is a non-nucleophilic organic base, preferably a non-nucleophilic organic base containing nitrogen.
75. The method as described in paragraph 74, wherein the nitrogen containing base is trimethylamine.
76. The method as described in paragraph 73, wherein the inorganic base is a carbonate salt, preferably K₂CO₃.
77. A method of preparing a PIM co-polymer, wherein the method includes the step of reacting together (i) a biscatechol monomer of Formula (V) or Formula (VII), (ii) a first suitable linking monomer, and (iii) a second suitable linking monomer that is different to the first suitable linking monomer.
78. The method as described in paragraph 77, wherein the first and second suitable linking monomers are tetrahalo monomers selected from a tetrafluoro, tetrabromo, tetrachloro or tetraiodo aromatic monomer.
79. The method as described in paragraph 77 or 78, wherein the first biscatechol monomer, is reacted with the first and second suitable linking monomers in the presence of an organic base, an inorganic base and/or fluoride ions.
80. The method as described in paragraph 79, wherein the biscatechol monomer is of Formula (V) and is base sensitive and is reacted with the first and second suitable linking monomers in the presence of fluoride ions.
81. The method as described in paragraph 80, wherein the fluoride ions are provided by organic or inorganic fluoride ion sources, wherein the fluoride ions are sourced from any one or more of potassium fluoride (KF), tetrabutylammonium fluoride (TBAF), cesium fluoride (CsF), organic quaternary ammonium fluoride salts, and other inorganic fluoride salts.
82. The method as described in paragraph 79, wherein the biscatechol monomer is not base sensitive and is reacted with the first and second suitable linking monomers in the presence of an organic or inorganic base.
83. The method as described in paragraph 82, wherein the organic base is a non-nucleophilic organic base, preferably a non-nucleophilic organic base containing nitrogen.
84. The method as described in paragraph 83, wherein the nitrogen containing base is trimethylamine.
85. The method as described in paragraph 82, wherein the inorganic base is a carbonate salt, preferably K₂CO₃.
86. A PIM homo- or co-polymer including at least one biscatechol monomer of Formula (VIII):

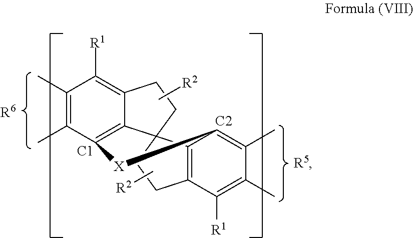

wherein
each R¹ can be the same or different, each R² can be the same or different and wherein R¹ and/or R² are selected from any one or more of H; straight or branched, saturated or unsaturated lower C₁-C₆ alkyl groups, wherein the lower alkyl groups can include aromatic or non-aromatic ring structures R³OR⁴, R³O(C═O)R⁴, R³C(═O)OR⁴, or R³OH, wherein each of R³ and R⁴ are the same or different and are selected from straight or branched, saturated or unsaturated lower C₁-C₆ alkyl groups; and
R⁵ and R⁶ represent suitable linking monomers, wherein the linking monomers can be the same or different and are preferably selected from any one or more tetrahalo aromatic monomers;
   (i) X is —CH₂—Y—CH₂—, and wherein Y is O; CH₂; C(═O); C(OR⁷)(OR⁸); NH; NR⁹; S; S(═O); SO₂; C(═S); PH; PR¹⁰; BH; BR¹¹ or BOR¹¹;
      and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or
   (ii) X is —CH₂-CH₂—, —CH═CH—, —(═O)O—, —C(═O)NH— or —CHR¹²-CHR¹²—, wherein R¹² are selected from any one or more of H or straight or branched, saturated or unsaturated lower C₁-C₆ alkyl groups, wherein the lower alkyl groups can include aromatic or non-aromatic ring structures.
87. The PIM homo- or co-polymer as described in paragraph 86, wherein each R² is a dimethyl and the biscatechol monomer contains a fused SBI ring system.
88. The biscatechol monomer as described in paragraph 86, wherein each R² is a C₆ aromatic ring and the biscatechol monomer contains a fused SBF ring system.
89. A method of increasing the rigidity of PIM homo- or co-polymers including repeating units containing at least one biscatechol monomer having a fused spiro-bisindane ring system (SBI) of Formula (I), the SBI ring system including a bicyclic spiro-carbon:

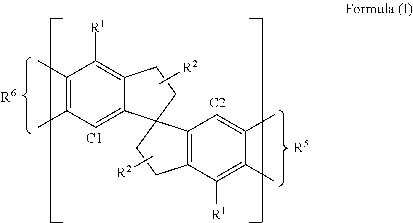

wherein
each R¹ can be the same or different, each R² can be the same or different and wherein R¹ and/or R² are selected from any one or more of H; straight or branched, saturated or unsaturated lower C₁-C₆ alkyl groups, wherein the lower alkyl groups can include aromatic or non-aromatic ring structures; R³OR⁴, R³O(C═O)R⁴, R³C(═O)OR⁴, or R³OH, wherein each of R³ and R⁴ are the same or different and are selected from straight or branched, saturated or unsaturated lower C₁-C₆ alkyl groups; and
R⁵ and R⁶ represent suitable linking monomers, wherein the linking monomers can be the same or different and are preferably selected from any one or more tetrahalo aromatic monomers;
and wherein the method includes the step of introducing an intra-molecular lock between C1and C2 of the biscatechol monomer of Formula (I), wherein the intramolecular lock between C1 and C2 forms:
   (a) an 8 membered ring structure including —CH₂—Y—CH₂—, and wherein Y is O; CH₂; C(═O); C(OR⁷)(OR⁸); NH; NR⁹; S; S(═O); SO₂; C(═S); PH; PR¹⁰; BH; BR¹¹ or BOR¹¹; and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups,
      wherein the lower alkyl groups can include aromatic or non-aromatic ring structures; or
   (b) a 7 membered ring structure including —CH₂-CH₂—, —CH═CH—, —(═O)O—, —C(═O)NH— or —CHR¹²-CHR¹²—, wherein R¹²are selected from any one or more of H or straight or branched, saturated or unsaturated lower C₁-C₆ alkyl groups, wherein the lower alkyl groups can include aromatic or non-aromatic ring structures.
90. A method of increasing the rigidity of PIM homo- or co-polymers including repeating units containing at least one biscatechol monomer having a fused spiro-bisindane ring system (SBI) of Formula (I), the SBI ring system including a bicyclic spiro-carbon:

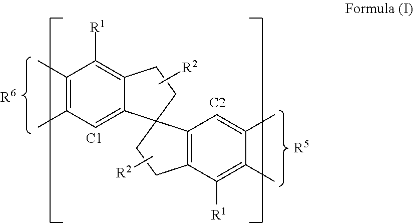

wherein each R¹ is H, and wherein each R² is dimethyl and the biscatechol monomer of Formula (I) is:

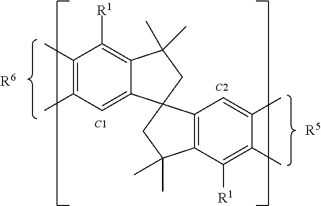

wherein the biscatechol monomer contains a fused spiro-bisindane ring system (SBI); and
R⁵ and R⁶ represent suitable linking monomers, selected from any one or more tetrahalo aromatic monomers;
and wherein the method includes the step of introducing an intra-molecular lock between C1 and C2 of the biscatechol monomer of Formula (I), wherein the intramolecular lock between C1 and C2 forms:
   (a) an 8 membered ring structure including —CH₂—Y—CH₂—, and wherein Y is O; CH₂; C(═O); C(OR⁷)(OR⁸); NH; NR⁹; S; S(═O); SO₂; C(═S); PH; PR¹⁰; BH; BR¹¹ or BOR¹¹; and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or
   (b) a 7 membered ring structure including —CH₂-CH₂—, —CH═CH—, —(═O)O—, —C(═O)NH— or —CHR¹²-CHR¹²—, wherein R¹²are selected from any one or more of H or straight or branched, saturated or unsaturated lower C₁-C₆ alkyl groups, wherein the lower alkyl groups can include aromatic or non-aromatic ring structures.
91. A method of increasing the rigidity of PIM homo- or co-polymers including repeating units containing at least one biscatechol monomer having a fused spiro-bisindane ring system (SBI) of Formula (I), the SBI ring system including a bicyclic spiro-carbon:

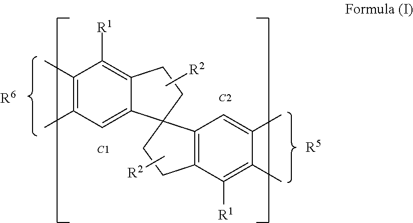

wherein each R¹ is H, and wherein each R² is wherein each R² is a C₆ aromatic ring and the biscatechol monomer of Formula (I) is:

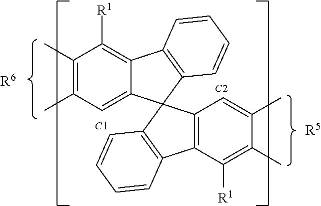

wherein the biscatechol monomer contains a fused spiro-bisfluorene ring system (SBF); and
R⁵ and R⁶ represent suitable linking monomers, selected from any one or more tetrahalo aromatic monomers;
and wherein the method includes the step of introducing an intra-molecular lock between C1 and C2 of the biscatechol monomer of Formula (I), wherein the intramolecular lock between C1 and C2 forms: an 8 membered ring structure including —CH₂—Y—CH₂—, and wherein Y is O; CH₂; C(═O); C(OR⁷)(OR⁸); NH; NR⁹; S; S(═O); SO₂; C(═S); PH; PR¹⁰; BH; BR¹¹ or BOR¹¹; and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or a 7 membered ring structure including —CH₂-CH₂—, —CH═CH—, —(═O)O—, —C(═O)NH— or —CHR¹²-CHR¹²—, wherein R¹² are selected from any one or more of H or straight or branched, saturated or unsaturated lower C₁-C₆ alkyl groups, wherein the lower alkyl groups can include aromatic or non-aromatic ring structures.
92. A method of increasing the rigidity of PIM homo- or co-polymers including repeating units containing at least one biscatechol monomer having a fused spiro-bisindane ring system (SBI) of Formula (I), the SBI ring system including a bicyclic spiro-carbon:

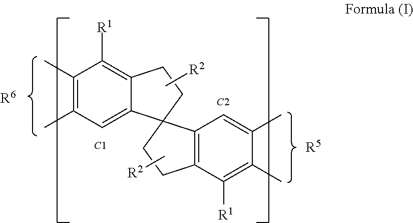

wherein each R¹ can be the same or different, each R² can be the same or different and wherein R¹ and/or R² are selected from any one or more of H; straight or branched, saturated or unsaturated lower C₁-C₆ alkyl groups, wherein the lower alkyl groups can include aromatic or non-aromatic ring structures; R³OR⁴, R³O(C═O)R⁴, R³C(═O)OR⁴, or R³OH, wherein each of R³ and R⁴ are the same or different and are selected from straight or branched, saturated or unsaturated lower C₁-C₆ alkyl groups; and
R⁵ and R⁶ represent suitable linking monomers selected from any one or more of tetrafluoro, tetrabromo, tetrachloro or tetraiodo aromatic monomers (and in particular tetrafluoro aromatic monomers);
and wherein the method includes the step of introducing an intra-molecular lock between C1 and C2 of the biscatechol monomer of Formula (I), wherein the intramolecular lock between C1 and C2 forms:
   (a) an 8 membered ring structure including —CH₂—Y—CH₂—, and wherein Y is O; CH₂; C(═O); C(OR⁷)(OR⁸); NH; NR⁹; S; S(═O); SO₂; C(═S); PH; PR¹⁰; BH; BR¹¹ or BOR¹¹; and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or
   (b) a 7 membered ring structure including —CH₂-CH₂—, —CH═CH—, —(═O)O—, —C(═O)NH— or —CHR¹²-CHR¹²—, wherein R¹²are selected from any one or more of H or straight or branched, saturated or unsaturated lower C₁-C₆ alkyl groups,
wherein the lower alkyl groups can include aromatic or non-aromatic ring structures.
93. A method of preparing a biscatechol monomer of Formula (V), the biscatechol monomer including a fused spiro-bisindane ring system (SBI) having a locked bicyclic spiro-carbon, wherein the method includes the steps of:
   (a) protecting each hydroxyl group of a dimethyl substituted biscatechol monomer of Formula (II) having a bicyclic spiro-carbon with a silyl ether protecting group to give a protected dimethyl substituted biscatechol monomer of Formula (III):

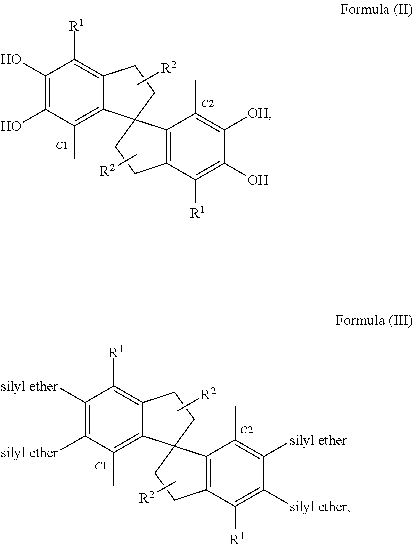

wherein the silyl ether protecting groups are selected from any one or more of trimethylsilyl (TMS), tert-butyldiphenylsilyl (TBDPS), tert-butyldimethylsilyl (TBS) and triisopropylsilyl (TIPS); and
wherein, each R1 can be the same or different, each R2 can be the same or different and wherein R1 and/or R2 are selected from any one or more of H; straight or branched, saturated or unsaturated lower C1-C6 alkyl groups, wherein the lower alkyl groups can include aromatic or non-aromatic ring structures;
R3OR4, R30(C═O)R4, R3C(═O)OR4, or R3OH, wherein each of R3 and R4 are the same or different and are selected from straight or branched, saturated or unsaturated lower C1-C6 alkyl groups; and
(b) halogenating the silyl ether protected biscatechol monomer of Formula (III) from step (a) to form a silyl ether protected bis(halomethyl) substituted biscatechol monomer of Formula (IV), wherein the halide ion employed in the halogenation is selected from bromide, chloride or iodide ions:

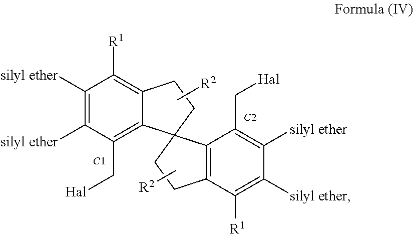

wherein R¹ and R² and silyl ether are as previously defined, and wherein Hal is any one of bromide, chloride or iodide ions, and
(c) (i) dehalogenating the biscatechol monomer of Formula (IV) and forming a covalent, intramolecular lock between C1 and C2 of the protected biscatechol monomer of Formula (IV) with a suitable locking group X; wherein the dehalogenation step is catalysed by a transition metal salt (in particular a silver(I), iron(III), titanium(II) or tin(II) salt), metal oxide (in particular ZnO or Ag₂O) and/or a pure metal (in particular zinc); or
(ii) substituting the halide ions in Formula (IV) with hydroxyl groups, followed by cyclised dehydration to form a covalent, intramolecular lock between C1 and C2 of the protected biscatechol monomer of Formula (IV);
to provide a silyl ether protected biscatechol monomer of Formula (V) having a locked bicyclic spiro-carbon:

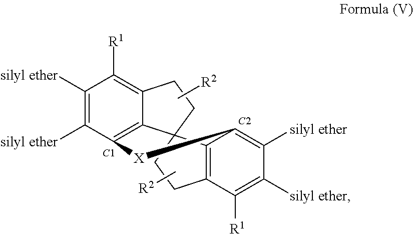

wherein, R1 and R2 and silyl ether are as previously defined, and
   (d) (i) X is —CH₂—Y—CH₂—, and wherein Y is O; CH₂; C(═O); C(OR⁷)(OR⁸); NH; NR⁹; S; S(═O); SO₂; C(═S); PH; PR¹⁰; BH; BR¹¹ or BOR¹¹;
      and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or
      (ii) X is —CH₂-CH₂—, —CH═CH—, —(═O)O—, —C(═O)NH— or —CHR¹²-CHR¹²—, wherein R¹² are selected from any one or more of H or straight or branched, saturated or unsaturated lower C₁-C₆ alkyl groups, wherein the lower alkyl groups can include aromatic or non-aromatic ring structures.
94. A method for preparing a biscatechol monomer of Formula (V), the biscatechol monomer including a fused spiro-bisindane ring system (SBI) having a bicyclic spiro-carbon, wherein the alternative method includes the steps of:
   (a) halogenating a silyl ether protected biscatechol monomer of Formula (III) to form a silyl ether protected bis(halomethyl) substituted biscatechol monomer of Formula (IV), wherein the halide ion employed in the halogenation is selected from bromide, chloride or iodide ions:

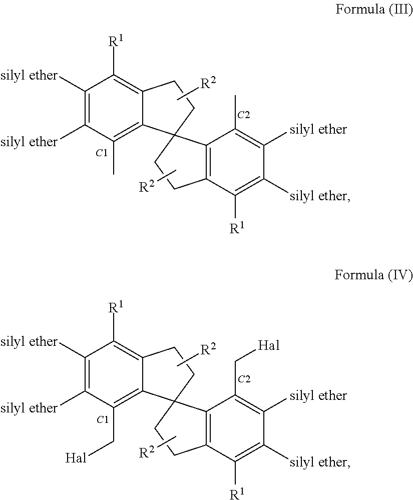

wherein, the silyl ether protecting groups are selected from any one or more of trimethylsilyl (TMS), tert-butyldiphenylsilyl (TBDPS), tert-butyldimethylsilyl (TBS) and triisopropylsilyl (TIPS); and
wherein, each R1 can be the same or different, each R2 can be the same or different and wherein R1 and/or R2 are selected from any one or more of H; straight or branched, saturated or unsaturated lower C1-C6 alkyl groups, wherein the lower alkyl groups can include aromatic or non-aromatic ring structures; R3OR4, R30(C═O)R4, R3C(═O)OR4, or R3OH, wherein each of R3 and R4 are the same or different and are selected from straight or branched, saturated or unsaturated lower C1-C6 alkyl groups; and wherein Hal is any one of bromide, chloride or iodide ions, and
(b) (i) dehalogenating the biscatechol monomer of Formula (IV) and forming a covalent, intramolecular lock between C1 and C2 of the protected biscatechol monomer of Formula (IV) with a suitable locking group X; wherein the dehalogenation step is catalysed by a transition metal salt (in particular a silver(I), iron(III), titanium(II) or tin(II) salt), metal oxide (in particular ZnO or Ag₂O) and/or a pure metal (in particular zinc); or
(ii) substituting the halide ions in Formula (IV) with hydroxyl groups, followed by cyclised dehydration to form a covalent, intramolecular lock between C1 and C2 of the protected biscatechol monomer of Formula (IV);
to provide a silyl ether protected biscatechol monomer of Formula (V) having a locked bicyclic spiro-carbon:

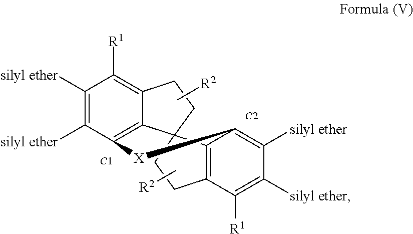

wherein, R¹ and R² and silyl ether are as previously defined, and
   (c) (i) X is —CH₂—Y—CH₂—, and wherein Y is O; CH₂; C(═O); C(OR⁷)(OR⁸); NH; NR⁹; S; S(═O); SO₂; C(═S); PH; PR¹⁰; BH; BR¹¹ or BOR¹¹;
      and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or
      (ii) X is —CH₂-CH₂—, —CH═CH—, —(═O)O—, —C(═O)NH— or —CHR¹²-CHR¹²—, wherein R¹² are selected from any one or more of H or straight or branched, saturated or unsaturated lower C₁-C₆ alkyl groups, wherein the lower alkyl groups can include aromatic or non-aromatic ring structures.
95. A method of preparing a biscatechol monomer of Formula (V), the biscatechol monomer including a fused spiro-bisindane ring system (SBI) having a locked bicyclic spiro-carbon, wherein the method includes the steps of:
   (I)
      (a) protecting each hydroxyl group of a dimethyl substituted biscatechol monomer of Formula (II) having a bicyclic spiro-carbon with a silyl ether protecting group to give a protected dimethyl substituted biscatechol monomer of Formula (III):

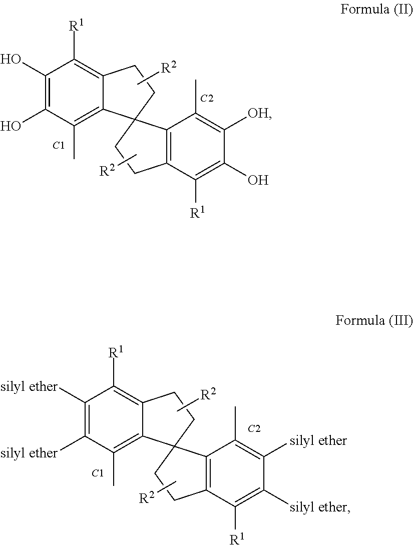

wherein the silyl ether protecting groups are selected from any one or more of trimethylsilyl (TMS), tert-butyldiphenylsilyl (TBDPS), tert-butyldimethylsilyl (TBS) and triisopropylsilyl (TIPS); and
wherein, R¹ is as defined previously for Formula (I) and wherein each R² is a dimethyl and the biscatechol monomer of Formula (V) contains a fused SBI ring system or each R² is a C₆ aromatic ring and the biscatechol monomer of Formula (V) contains a fused SBF ring system;
(b) halogenating the silyl ether protected biscatechol monomer of Formula (III) from step (a) to form a silyl ether protected bis(halomethyl) substituted biscatechol monomer of Formula (IV), wherein the halide ion employed in the halogenation is selected from bromide, chloride or iodide ions:

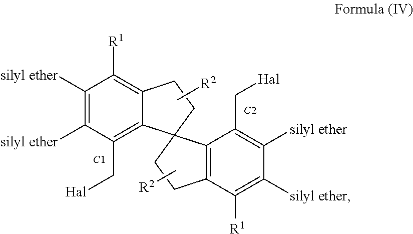

wherein R¹ and R² and silyl ether are as previously defined, and wherein Hal is any one of bromide, chloride or iodide ions, and
(c) (i) dehalogenating the biscatechol monomer of Formula (IV) and forming a covalent, intramolecular lock between C1 and C2 of the protected biscatechol monomer of Formula (IV) with a suitable locking group X; or
   (ii) substituting the halide ions in Formula (IV) with hydroxyl groups, followed by cyclised dehydration to form a covalent, intramolecular lock between C2 and C2 of the protected biscatechol monomer of Formula (IV);
   to provide a silyl ether protected biscatechol monomer of Formula (V) having a locked bicyclic spiro-carbon:

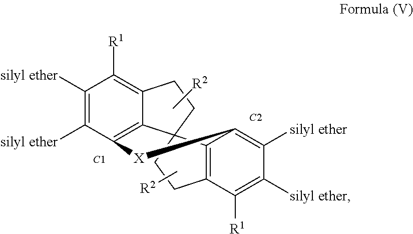

wherein, R¹ and R² and silyl ether are as previously defined, and
(d) (i) X is —CH₂—Y—CH₂—, and wherein Y is O; CH₂; C(═O); C(OR⁷)(OR⁸); NH; NR⁹; S; S(═O); SO₂; C(═S); PH; PR¹⁰; BH; BR¹¹ or BOR¹¹;
   and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or

(ii) X is —CH₂-CH₂—, —CH═CH—, —(═O)O—, —C(═O)NH— or —CHR¹²-CHR¹²—, wherein R¹² are selected from any one or more of H or straight or branched, saturated or unsaturated lower C₁-C₆ alkyl groups, wherein the lower alkyl groups can include aromatic or non-aromatic ring structures; or
(II)
   (a) halogenating a silyl ether protected biscatechol monomer of Formula (III) to form a silyl ether protected bis(halomethyl) substituted biscatechol monomer of Formula (IV), wherein the halide ion employed in the halogenation is selected from bromide, chloride or iodide ions:

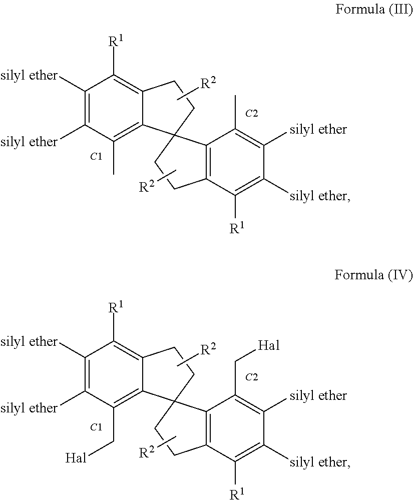

wherein, the silyl ether protecting groups are selected from any one or more of trimethylsilyl (TMS), tert-butyldiphenylsilyl (TBDPS), tert-butyldimethylsilyl (TBS) and triisopropylsilyl (TIPS), and
wherein, R¹ is as defined previously for Formula (I) and wherein each R² is a dimethyl and the biscatechol monomer of Formula (V) contains a fused SBI ring system or each R² is a C₆ aromatic ring and the biscatechol monomer of Formula (V) contains a fused SBF ring system; and wherein Hal is any one of bromide, chloride or iodide ions, and
(b) (i) dehalogenating the biscatechol monomer of Formula (IV) and forming a covalent, intramolecular lock between C1 and C2 of the protected biscatechol monomer of Formula (IV) with a suitable locking group X; or
   (ii) substituting the halide ions in Formula (IV) with hydroxyl groups, followed by cyclised dehydration to form a covalent, intramolecular lock between C1 and C2 of the protected biscatechol monomer of Formula (IV);
   to provide a silyl ether protected biscatechol monomer of Formula (V) having a locked bicyclic spiro-carbon:

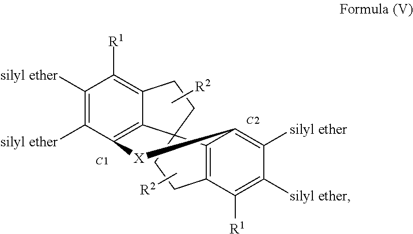

wherein, R¹ and R² and silyl ether are as previously defined, and
(c) (i) X is —CH₂—Y—CH₂—, and wherein Y is O; CH₂; C(═O); C(OR⁷)(OR⁸); NH; NR⁹; S; S(═O); SO₂; C(═S); PH; PR¹⁰; BH; BR¹¹ or BOR¹¹;
   and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or
   (ii) X is —CH₂-CH₂—, —CH═CH—, —(═O)O—, —C(═O)NH— or —CHR¹²-CHR¹²—, wherein R¹² are selected from any one or more of H or straight or branched, saturated or unsaturated lower C₁-C₆ alkyl groups, wherein the lower alkyl groups can include aromatic or non-aromatic ring structures.
96. A silyl ether protected biscatechol monomer of Formula (IV), the biscatechol monomer including a fused spiro-bisindane ring system (SBI) having a bicyclic spiro-carbon:

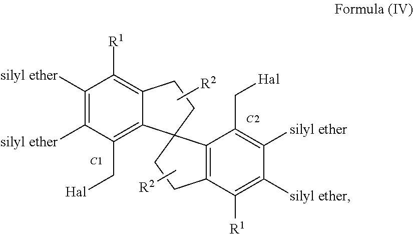

wherein
each R¹ can be the same or different, each R² can be the same or different and wherein R¹ and/or R² are selected from any one or more of H; straight or branched, saturated or unsaturated lower C₁-C₆ alkyl groups, wherein the lower alkyl groups can include aromatic or non-aromatic ring structures R³OR⁴, R³O(C═O)R⁴, R³C(═O)OR⁴, or R³OH, wherein each of R³ and R⁴ are the same or different and are selected from straight or branched, saturated or unsaturated lower C₁-C₆ alkyl groups; Hal represents any one of bromide, chloride or iodide ions; and
wherein the silyl ether protecting groups are selected from any one or more of trimethylsilyl (TMS), tert-butyldiphenylsilyl (TBDPS), tert-butyldimethylsilyl (TBS) and triisopropylsilyl (TIPS).
97. A silyl ether protected biscatechol monomer of Formula (V), the biscatechol monomer including a fused spiro-bisindane ring system (SBI) having a bicyclic spiro-carbon:

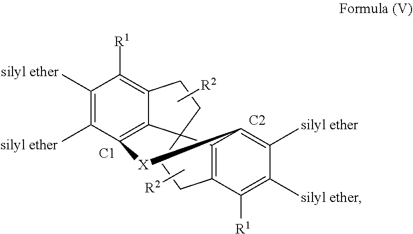

wherein,
each R¹ can be the same or different and is selected from any one or more of H; straight or branched, saturated or unsaturated lower C₁-C₆ alkyl groups, wherein the lower alkyl groups can include aromatic or non-aromatic ring structures R³OR⁴, R³O(C═O)R⁴, R³C(═O)OR⁴, or R³OH, wherein each of R³ and R⁴ are the same or different and are selected from straight or branched, saturated or unsaturated lower C₁-C₆ alkyl groups;

and
wherein each R² is a dimethyl and the biscatechol monomer contains a fused SBI ring system, or wherein each R² is a C₆ aromatic ring and the biscatechol monomer contains a fused SBF ring system; and
wherein,
   (i) X is —CH₂—Y—CH₂—, and wherein Y is O; CH₂; C(═O); C(OR⁷)(OR⁸); NH; NR⁹; S; S(═O); SO₂; C(═S); PH; PR¹⁰; BH; BR¹¹ or BOR¹¹;
      and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or
   (ii) X is —CH₂-CH₂—, —CH═CH—, —(═O)O—, —C(═O)NH— or —CHR¹²-CHR¹²—, wherein R¹² are selected from any one or more of H or straight or branched, saturated or unsaturated lower C₁-C₆ alkyl groups, wherein the lower alkyl groups can include aromatic or non-aromatic ring structures ; and
      wherein the silyl ether protecting group is selected from any one or more of trimethylsilyl (TMS), tert-butyldiphenylsilyl (TBDPS), tert-butyldimethylsilyl (TBS) and triisopropylsilyl (TIPS).
98. A method of preparing a biscatechol monomer of Formula (VII), the biscatechol monomer including a fused spiro-bisindane ring system (SBI) having a locked bicyclic spiro-carbon, wherein the method includes the steps of deprotecting a silyl ether protected biscatechol monomer of Formula (V) using tetrabutylammonium fluoride (TBAF) to form a biscatechol monomer of Formula (VII) having a locked bicyclic carbon:

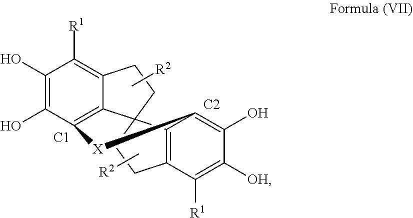

wherein,
   (i) X is —CH₂—Y—CH₂—, and wherein Y is O; CH₂; C(═O); C(OR⁷)(OR⁸); NH; NR⁹; S; S(═O); SO₂; C(═S); PH; PR¹⁰; BH; B R¹¹ or BO R¹¹;
      and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or
   (ii) X is —CH₂-CH₂—, —CH═CH—, —(═O)O—, —C(═O)NH— or —CHR¹²-CHR¹²—, wherein R¹² are selected from any one or more of H or straight or branched, saturated or unsaturated lower C₁-C₆ alkyl groups, wherein the lower alkyl groups can include aromatic or non-aromatic ring structures; and
   (iii) R¹ is as defined for Formula (V), and each R2 is a dimethyl and the biscatechol monomer contains a fused SBI ring system, or each R² is a C₆ aromatic ring and the biscatechol monomer contains a fused SBF ring system.
99. A biscatechol monomer of Formula (VII), the biscatechol monomer including a fused spiro-bisindane ring system (SBI) having a locked bicyclic spiro-carbon:

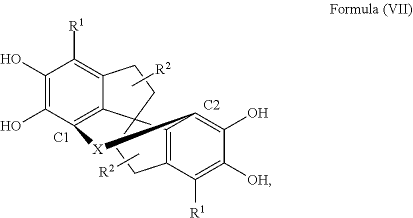

wherein,
each R¹ can be the same or different and is selected from any one or more of H; straight or branched, saturated or unsaturated lower C₁-C₆ alkyl groups, wherein the lower alkyl groups can include aromatic or non-aromatic ring structures R³OR⁴, R³O(C═O)R⁴, R³C(═O)OR⁴, or R³OH, wherein each of R³ and R⁴ are the same or different and are selected from straight or branched, saturated or unsaturated lower C₁-C₆ alkyl groups;
and
each R² is a dimethyl and the biscatechol monomer contains a fused SBI ring system, or each R² is a C₆ aromatic ring and the biscatechol monomer contains a fused SBF ring system; and
   (i) X is —CH₂—Y—CH₂—, and wherein Y is O; CH₂; C(═O); C(OR⁷)(OR⁸); NH; NR⁹; S; S(═O); SO₂; C(═S); PH; PR¹⁰; BH; BR¹¹ or BOR¹¹;
      and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or
   (ii) X is —CH₂-CH₂—, —CH═CH—, —(═O)O—, —C(═O)NH— or —CHR¹²-CHR¹²—, wherein R¹² are selected from any one or more of H or straight or branched, saturated or unsaturated lower C₁-C₆ alkyl groups, wherein the lower alkyl groups can include aromatic or non-aromatic ring structures.
100. A method for the synthesis of a PIM polymer, the method including a fluoride-mediated double nucleophilic aromatic substitution polymerization of a biscatechol monomer of any of Formulae (III), (IV) or (V) with a suitable linking monomer selected from a tetrafluoro, tetrabromo, tetrachloro or tetraiodo aromatic monomer (in particular 2,3,5,6-tetrafluoroterephthalonitrile); and
   wherein the silyl ether protecting groups on biscatechol monomer of Formulae (III), (IV) or (V) can be the same or different and are selected from any one or more of trimethylsilyl (TMS), tert-butyldiphenylsilyl (TBDPS), tert-butyldimethylsilyl (TBS) and triisopropylsilyl (TIPS); and
   wherein the fluoride ions for the fluoride-mediated polymerization are sourced from any one or more of potassium fluoride (KF), tetrabutylammonium fluoride (TBAF), cesium fluoride (CsF), organic quaternary ammonium fluoride salts, phosphonium fluoride and other inorganic fluoride salts; and
   wherein there is at least a 0.001 molar ratio equivalence of fluoride ions to silyl ether protecting groups present on the biscatechol monomer of Formulae (III), (IV) or (V) to catalyse the reaction between the biscatechol monomer and the suitable linking monomer.
101. A method of preparing a PIM homo-polymer, wherein the method includes the step of reacting a base sensitive biscatechol monomer of Formulae (III), (IV) or (V) with a suitable linking monomer selected from a tetrafluoro, tetrabromo, tetrachloro or tetraiodo aromatic monomer (in particular 2,3,5,6-tetrafluoroterephthalonitrile), in the presence of fluoride ions sourced from any one or more of potassium fluoride (KF), tetrabutylammonium fluoride (TBAF), cesium fluoride (CsF), organic quaternary ammonium fluoride salts, and other inorganic fluoride salts; and there is at least a 0.001 molar ratio equivalence (and in particular about 0.001 to about 3 or 4) of fluoride ions to silyl ether protecting groups present on the biscatechol monomer of Formulae (III), (IV) or (V) to catalyse the reaction between the biscatechol monomer and the suitable linking monomer.
102. A method of preparing a PIM co-polymer, wherein the method includes the step of reacting together (i) a first base sensitive biscatechol monomer of Formulae (III), (IV) or (V), (ii) a second biscatechol monomer that is different to the first biscatechol monomer, and (iii) a suitable linking monomer selected from a tetrafluoro, tetrabromo, tetrachloro or tetraiodo aromatic monomer (in particular 2,3,5,6-tetrafluoroterephthalonitrile), in the presence of fluoride ions sourced from any one or more of potassium fluoride (KF), tetrabutylammonium fluoride (TBAF), cesium fluoride (CsF), organic quaternary ammonium fluoride salts, and other inorganic fluoride salts; and there is at least a 0.001 molar ratio equivalence (and in particular about 0.001 to about 3 or 4) of fluoride ions to silyl ether protecting groups present on the biscatechol monomer of Formulae (III), (IV) or (V) to catalyse the reaction between the biscatechol monomer and the suitable linking monomer.
103. A method of preparing a PIM co-polymer, wherein the method includes the step of reacting together (i) a base sensitive biscatechol monomer of Formulae (III), (IV) or (V), (ii) a first suitable linking monomer, and (iii) a second suitable linking monomer that is different to the first suitable linking monomer; wherein the first and second suitable linking monomers are tetrahalo monomers selected from a tetrafluoro, tetrabromo, tetrachloro or tetraiodo aromatic monomer, in the presence of fluoride ions sourced from any one or more of potassium fluoride (KF), tetrabutylammonium fluoride (TBAF), cesium fluoride (CsF), organic quaternary ammonium fluoride salts, and other inorganic fluoride salts; and there is at least a 0.001 molar ratio equivalence (and in particular about 0.001 to about 3 or 4) of fluoride ions to silyl ether protecting groups present on the biscatechol monomer of Formulae (III), (IV) or (V) to catalyse the reaction between the biscatechol monomer and the suitable linking monomers.
104. A method of preparing a PIM homo-polymer, wherein the method includes the step of reacting a non-base sensitive biscatechol monomer of Formulae (III), (IV), (V) or (VII) with a suitable linking monomer selected from a tetrafluoro, tetrabromo, tetrachloro or tetraiodo aromatic monomer, in the presence of trimethylamine or K₂CO₃.
105. A method of preparing a PIM co-polymer, wherein the method includes the step of reacting together (i) a first non-base sensitive biscatechol monomer of Formulae (III), (IV), (V) or (VII), (ii) a second non-base sensitive biscatechol monomer that is different to the first biscatechol monomer (but which can be selected from a non-base sensitive biscatechol monomer of Formulae (III), (IV), (V) and/or (VII)), and (iii) a suitable linking monomer selected from a tetrafluoro, tetrabromo, tetrachloro or tetraiodo aromatic monomer, in the presence of trimethylamine or K₂CO₃.
106. A method of preparing a PIM co-polymer, wherein the method includes the step of reacting together (i) a non-base sensitive biscatechol monomer of Formulae (III), (IV), (V) or (VII), (ii) a first suitable linking monomer, and (iii) a second suitable linking monomer that is different to the first suitable linking monomer; wherein the first and second suitable linking monomers are tetrahalo monomers selected from a tetrafluoro, tetrabromo, tetrachloro or tetraiodo aromatic monomer, in the presence of trimethylamine or K₂CO₃.
107. A PIM homo- or co-polymer including at least one biscatechol monomer of Formula (VIII):

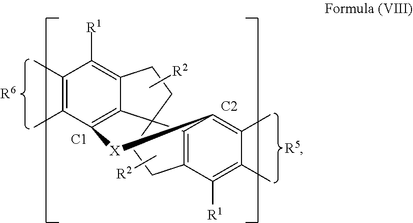

wherein
each R¹ can be the same or different and is selected from any one or more of H; straight or branched, saturated or unsaturated lower C₁-C₆ alkyl groups, wherein the lower alkyl groups can include aromatic or non-aromatic ring structures R³OR⁴, R³O(C═O)R⁴, R³C(═O)OR⁴, or R³OH, wherein each of R³ and R⁴ are the same or different and are selected from straight or branched, saturated or unsaturated lower C₁-C₆ alkyl groups;
and
each R² is a dimethyl and the biscatechol monomer contains a fused SBI ring system or each R² is a C₆ aromatic ring and the biscatechol monomer contains a fused SBF ring system; and
R⁵ and R⁶ represent suitable linking monomers, wherein the linking monomers can be the same or different and are tetrahalo aromatic monomers selected from any one or more of tetrafluoro, tetrabromo, tetrachloro or tetraiodo aromatic monomers;
   (i) X is —CH₂—Y—CH₂—, and wherein Y is O; CH₂; C(═O); C(OR⁷)(OR⁸); NH; NR⁹; S; S(═O); SO₂; C(═S); PH; PR¹⁰; BH; BR¹¹ or BOR¹¹;
      and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or
   (ii) X is —CH₂-CH₂—, —CH═CH—, —(═O)O—, —C(═O)NH— or —CHR¹²-CHR¹²—, wherein

R¹² are selected from any one or more of H or straight or branched, saturated or unsaturated lower C₁-C₆ alkyl groups, wherein the lower alkyl groups can include aromatic or non-aromatic ring structures.

108. A method of increasing the rigidity of PIM homo- or co-polymers including repeating units containing at least one biscatechol monomer having a fused spiro-bisindane ring system of Formula (I), the spiro-bisindane ring system (SBI) including a bicyclic spiro-carbon:

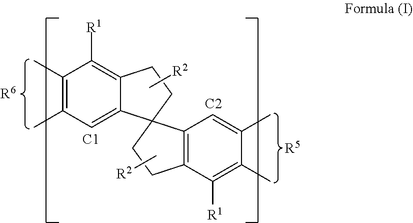

wherein
each R¹ can be the same or different, each R² can be the same or different and wherein R¹ and/or R² are selected from any one or more of H; straight or branched, saturated or unsaturated lower C₁-C₆ alkyl groups, wherein the lower alkyl groups can include aromatic or non-aromatic ring structures (and in particular wherein R² is an aromatic or non-aromatic ring structure formed by two or three methylene units between two available positions from the four possible positions on the two non-aromatic five membered carbon rings); R³OR⁴, R³O(C═O)R⁴, R³C(═O)OR⁴, or R³OH, wherein each of R³ and R⁴ are the same or different and are selected from straight or branched, saturated or unsaturated lower C₁-C₆ alkyl groups; and
R⁵ and R⁶ represent suitable linking monomers, wherein the linking monomers can be the same or different and are preferably selected from any one or more tetrahalo aromatic monomers;
and wherein the method includes the step of introducing an intra-molecular lock between C1 and C2 of the biscatechol monomer of Formula (I).

109. A biscatechol monomer of Formula (II):

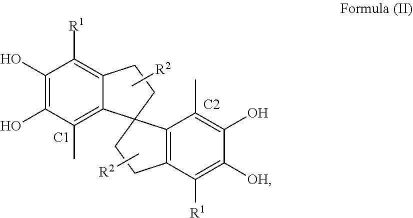

wherein each R¹ can be the same or different, each R² can be the same or different and wherein R¹ and/or R² are selected from any one or more of H; straight or branched, saturated or unsaturated lower C₁-C₆ alkyl groups, wherein the lower alkyl groups can include aromatic or non-aromatic ring structures (and in particular wherein R² is an aromatic or non-aromatic ring structure formed by two or three methylene units between two available positions from the four possible positions on the two non-aromatic five membered carbon rings); R³OR⁴, R³O(C═O)R⁴, R³C(═O)OR⁴, or R³OH, wherein each of R³ and R⁴ are the same or different and are selected from straight or branched, saturated or unsaturated lower C₁-C₆ alkyl groups.
110. A biscatechol monomer of Formula (III):

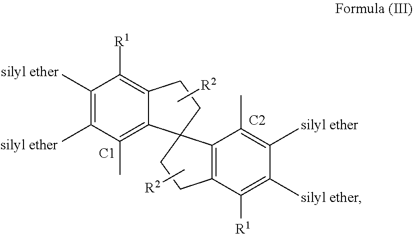

wherein each R¹ can be the same or different, each R² can be the same or different and wherein R¹ and/or R² are selected from any one or more of H; straight or branched, saturated or unsaturated lower C₁-C₆ alkyl groups, wherein the lower alkyl groups can include aromatic or non-aromatic ring structures (and in particular wherein R² is an aromatic or non-aromatic ring structure formed by two or three methylene units between two available positions from the four possible positions on the two non-aromatic five membered carbon rings); R³OR⁴, R³O(C═O)R⁴, R³C(═O)OR⁴, or R³OH, wherein each of R³ and R⁴ are the same or different and are selected from straight or branched, saturated or unsaturated lower C₁-C₆ alkyl groups.
111. A biscatechol monomer of any one of paragraphs 19, 21, 30, 109 and 110, or a homopolymer or copolymer including any one or more of those monomers, wherein R² is an aromatic or non-aromatic ring structure formed by two or three methylene units between two available positions from the four possible positions on the two non-aromatic five membered carbon rings.
112. A biscatechol monomer of Formula (II):

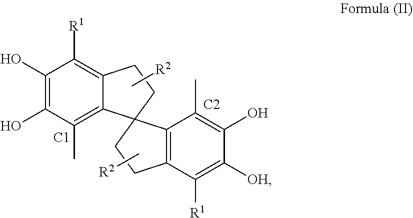

wherein each R¹ can be the same or different and is selected from any one or more of H; straight or branched, saturated or unsaturated lower C₁-C₆ alkyl groups, wherein the lower alkyl groups can include aromatic or non-aromatic ring structures; and wherein R² is an aromatic or non-aromatic ring structure formed by two or three methylene units between two available positions from the four possible positions on the two non-aromatic five membered carbon rings; R³OR⁴, R³O(C═O)R⁴, R³C(═O)OR⁴, or R³OH, wherein each of R³ and R⁴ are the same or different and are selected from straight or branched, saturated or unsaturated lower C₁-C₆ alkyl groups.
113. A biscatechol monomer of Formula (III):

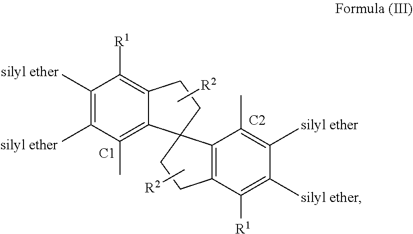

wherein each R¹ can be the same or different and is selected from any one or more of H; straight or branched, saturated or unsaturated lower C₁-C₆ alkyl groups, wherein the lower alkyl groups can include aromatic or non-aromatic ring structures; and wherein R² is an aromatic or non-aromatic ring structure formed by two or three methylene units between two available positions from the four possible positions on the two non-aromatic five membered carbon rings; R³OR⁴, R³O(C═O)R⁴, R³C(═O)OR⁴, or R³OH, wherein each of R³ and R⁴ are the same or different and are selected from straight or branched, saturated or unsaturated lower C₁-C₆ alkyl groups.

### REFERENCES

1. Budd, P. M.; Elabas, E. S.; Ghanem, B. S.; Makhseed, S.; McKeown, N. B.; Msayib, K. J.; Tattershall, C. E.; Wang, D. Adv. Mater. 2004, 16, 456.
2. McKeown, N. B.; Budd, P. M. Macromolecules 2010, 43, 5163-5176.
3. (a) Kricheldorf, H. R.; Fritsch, D.; Vakhtangishvili, L.; Schwarz, G. Macromol. Chem. Phys. 2005, 206, 2239-2247. (b) Kricheldorf, H. R.; Fritsch, D.; Vakhtangishvili, L.; Lomadze, N.; Schwarz, G. Macromolecules 2006, 39, 4990-4998.
4. Budd, P. M.; Msayib, K. J.; Tattershall, C. E.; Ghanem, B. S.; Reynolds, K. J.; McKeown, N. B.; Fritsch, D. J. Membr. Sci. 2005, 251, 263-269.
5. Budd, P. M.; Butler, A.; Selbie, J.; Mahmood, K.; McKeown, N. B.; Ghanem, B.; Msayib, K.; Book, D.; Walton, A. Phys. Chem. Chem. Phys. 2007, 9, 1802-1808.
6. Mckeown, N. B.; Ghanem, B. S.; Msayib, K. J.; Budd, P. M.; Tattershal, C. E.; Mahmoud, K.; Book, D.; Walton, A. Angew. Chem., Int. Ed. 2006, 45, 1804-1807.
7. Maffei, A. V.; Budd, P. M.; McKeown, N. B. Langmuir 2006, 22,4225-4229.
8. McKeown, N. B.; Budd, P. M.; Msayib, K. J.; Ghanem, B. S.; Kingston, H. J.; Tattershall, C. E.; Makhseed, S.; Reynolds, K. J.; Fritsch, D. Chem. Eur. J. 2005, 11, 2610-2620.
9. Budd, P. M.; Ghanem, B. S.; Makhseed, S.; McKeown, N. B.; Msayib, K. J.; Tattershall, C. E. Chem. Commun. 2004, 230-231.
10. Bezzu, C. G.; Carta, M.; Tonkins, A.; Jansen, J. C.; Bernardo, P.; Bazzarelli, F.; McKeown, N. B. Adv. Mater. 2012, 24, 5930-5933.
11. Carta, M.; Malpass-Evans, R.; Croad, M.; Rogan, Y.; Jansen, J. C.; Bernardo, P.; Bazzarelli, F.; McKeown, N. B. Science 2013, 339, 303-307.
12. B. D. Freeman, Macromolecules, 1999, 32, 375.
13. (a) Kricheldorf, H. R.; Bier, G. J. Polym. Sci. Polym. Chem. Ed. 1983, 21, 2283-2289. (b) Kricheldorf, H. R.; Bier, G. Polymer 1984, 25, 1151-1156. (c) Hedrick, J. L.; Brown, H. R.; Hofer, D. C.; Johnson, R. D. Macromolecules, 1989, 22,2048-2053. (d) Herbert, C. G.; Bass, R. G.; Watson, K. A.; Connell, J. W. Macromolecules, 1996, 29, 7709-7716.
14. Ten Hoeve, W.; Wynberg, H. J. Org. Chem. 1980, 45, (15) 2930-2937.
15. Fritsch, D.; Bengtson, G.; Carta, M.; McKeown, N. B. Macromol. Chem. Phys. 2011, 212, 1137-1146.
16. Zhang, J.; Jin, J.; Cooney, R.; Zhang, S. Polymer, 2015, 57,45-17. (a) Corey, E. J.; Venkateswarlu, A. J. Am. Chem. Soc. 1972, 94, 6190-6191. (b) Colvin, E. W. Chem. Soc. Rev. 1978, 7,15-64.
18. Kricheldorf, H. R. Silicon in Polymer Synthesis; Springer: Berlin, 1996.

1. A method of increasing the rigidity of a polymer of intrinsic microporosity (PIM), homo- or co-polymer& including repeating units containing at least one biscatechol monomer having a fused spiro-bisindane (SBI) ring system of Formula (I), the SBI ring system including a bicyclic spiro-carbon: 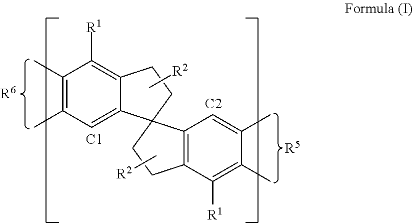
wherein
each R¹ can be the same or different, each R² can be the same or different and wherein R¹ and/or R² are selected from any one or more of H; straight or branched, saturated or unsaturated lower C₁-C₆ alkyl groups, wherein the lower alkyl groups can include aromatic or non-aromatic ring structures; R³OR⁴, R³O(C=O)R⁴, R³C(═O)OR⁴, or R³OH, wherein each of R³ and R⁴ are the same or different and are selected from straight or branched, saturated or unsaturated lower C₁-C₆ alkyl groups; and
R⁵ and R⁶ represent suitable linking monomers, wherein the linking monomers can be the same or different;
and wherein the method includes the step of introducing an intra-molecular lock between C1 and C2 of the biscatechol monomer of Formula (I).

2. The method of claim 1, wherein the intramolecular lock between C1 and C2 forms:
(a) an 8 membered ring structure including —CH₂—Y—CH₂—, and wherein Y is O; CH₂; C(═O); C(OR⁷)(OR⁸); NH; NR⁹; S; S(═O); SO₂; C(═S); PH; PR¹⁰; BH;
BR¹¹ or BOR¹¹; and wherein R⁷ and R⁸ are selected from any one or more of H or C₁-C₆ alkyl groups; and wherein R⁹, R¹⁰and R¹¹ are selected from any one or more of H or straight or branched, saturated or unsaturated lower C₁-C₆ alkyl groups, wherein the lower alkyl groups can include aromatic or non-aromatic ring structures; or
(b) a 7 membered ring structure including —CH₂-CH₂—, —CH═CH—, —(═O)O—, —C(═O)NH— or —CHR¹²-CHR¹²—, wherein R¹² are selected from any one or more of H or straight or branched, saturated or unsaturated lower C₁-C₆ alkyl groups, wherein the lower alkyl groups can include aromatic or non-aromatic ring structures.

3-15. (canceled)

16. A method of preparing a polymer of intrinsic microporosity homo-polymer or a co-polymer, wherein the method includes the step of
(a) reacting a biscatechol of Formula (V); 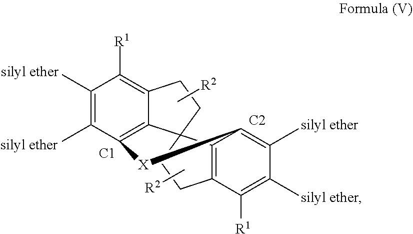
wherein
each R¹ can be the same or different, each R² can be the same or different and wherein R¹ and/or R² are selected from any one or more of H; straight or branched, saturated or unsaturated lower C₁-C₆ alkyl groups, wherein the lower alkyl groups can include aromatic or non-aromatic ring structures R³OR⁴, R³O(C═O)R⁴, R³C(═O)OR⁴, or R³OH, wherein each of R³ and R⁴ are the same or different and are selected from straight or branched, saturated or unsaturated lower C₁-C₆ alkyl groups; and
wherein,
(i) X is —CH₂—Y—CH₂—, and wherein Y is O; CH₂; C(═O); C(OR⁷)(OR⁸); NH; NR⁹; S; S(═O); SO₂; C(═S); PH; PR¹⁰; BH; BR¹¹ or BOR¹¹;
and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or
(ii) X is —CH₂-CH₂—, —CH═CH—, —(═O)O—, —C(═O)NH— or —CHR¹²-CHR¹²—, wherein R¹² are selected from any one or more of H or straight or branched, saturated or unsaturated lower C₁-C₆ alkyl groups, wherein the lower alkyl groups can include aromatic or non-aromatic ring structures ; and
wherein the silyl ether protecting groups are the same or different;
or Formula (VII); 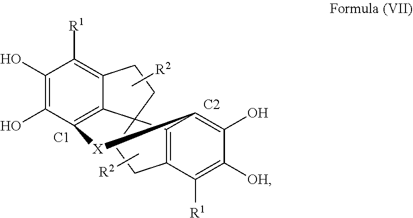
wherein
(i) X is —CH₂—Y—CH₂—, and wherein Y is O; CH₂; C(═O); C(OR⁷)(OR⁸); NH; NR⁹; S; S(═O); SO₂; C(═S); PH; PR¹⁰; BH; B R¹¹ or BO R¹¹;
and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or
(ii) X is —CH₂-CH₂—, —CH═CH—, —(═O)O—, —C(═O)NH— or —CHR¹²-CHR¹²—, wherein R¹² are selected from any one or more of H or straight or branched, saturated or unsaturated lower C₁-C₆ alkyl groups, wherein the lower alkyl groups can include aromatic or non-aromatic ring structures;
with a suitable tetrahalo aromatic linking monomer or
(b) reacting together a first biscatechol monomer of Formula (V) or Formula (VII), a second biscatechol monomer that is different to the first biscatechol monomer, and a suitable tetrahalo aromatic linking monomer, or
(c) reacting together a biscatechol monomer of Formula (V) or Formula (VII), a first suitable tetrahalo aromatic linking monomer, and a second suitable tetrahalo aromatic linking monomer that is different to the first suitable tetrahalo aromatic linking monomer,
wherein the monomers are reacted in the presence of an organic base, an inorganic base and/or fluoride ions.

17-18. (canceled)

19. The method of claims 16, wherein the biscatechol monomer is of Formula (V) and is base sensitive and is reacted with the suitable tetrahalo aromatic linking monomer in the presence of fluoride ions.

20. The method of claims 16, wherein the biscatechol monomer of Formula (V) or Formula (VII) is not base sensitive and is reacted with the suitable tetrahalo aromatic linking monomer in the presence of an organic or inorganic base.

21. A polymer of intrinsic microporosity (PIM) homo- or co-polymer including at least one biscatechol monomer of Formula (VIII): 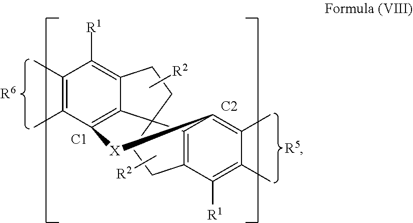
wherein
each R¹ can be the same or different, each R² can be the same or different and wherein R¹ and/or R² are selected from any one or more of H; straight or branched, saturated or unsaturated lower C₁-C₆ alkyl groups, wherein the lower alkyl groups can include aromatic or non-aromatic ring structures R³OR⁴, R³O(C═O)R⁴, R³C(═O)OR⁴, or R³OH, wherein each of R³ and R⁴ are the same or different and are selected from straight or branched, saturated or unsaturated lower C₁-C₆ alkyl groups; and
R⁵ and R⁶ represent suitable linking monomers, wherein the linking monomers can be the same or different and are preferably selected from any one or more tetrahalo aromatic monomers;
(i) X is —CH₂—Y—CH₂—, and wherein Y is O; CH₂; C(═O); C(OR⁷)(OR⁸); NH; NR⁹; S; S(═O); SO₂; C(═S); PH; PR¹⁰; BH; BR¹¹ or BOR¹¹;
and wherein R⁷ and R⁸ are selected from any one or more of H or C₁-C₆ alkyl groups; and wherein R⁹, R¹⁰ and R¹¹ are selected from any one or more of H or straight or branched, saturated or unsaturated lower C₁-C₆ alkyl groups, wherein the lower alkyl groups can include aromatic or non-aromatic ring structures; or
(ii) X is —CH₂-CH₂—, —CH═CH—, —(═O)O—, —C(═O)NH— or —CHR¹²-CHR¹²—, wherein R¹² are selected from any one or more of H or straight or branched, saturated or unsaturated lower C₁-C₆ alkyl groups, wherein the lower alkyl groups can include aromatic or non-aromatic ring structures.

22. The method of claim 1, wherein R⁵ and R⁶ represent suitable linking monomers selected from any one or more tetrahalo aromatic monomers.

23. The method of claim 1, wherein each R² is dimethyl and the biscatechol monomer of Formula (I) is: 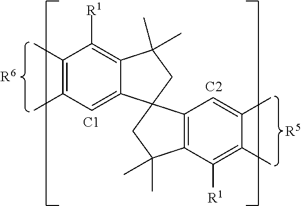
wherein the biscatechol monomer contains a fused spiro-bisindane ring system (SBI).

24. The method of claim 23, wherein each R¹ is H.

25. The method of claim 1, wherein each R² is a C₆ aromatic ring and the biscatechol monomer of Formula (I) is: 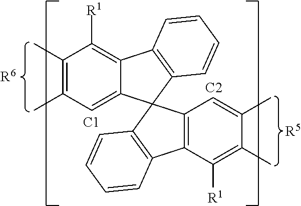
wherein the biscatechol monomer contains a fused spiro-bisfluorene ring system (SBF).

26. The method of claim 1, wherein the suitable tetrahalo aromatic monomer is a tetrafluoro, tetrabromo, tetrachloro or tetraiodo aromatic monomer.

27. The method of claim 26, wherein the tetrahalo aromatic monomer is a tetrafluoro aromatic monomer.
